(19) United States
(12) Patent Application Publication
(10) Pub. No.: US 2020/0023018 A1
(43) Pub. Date: Jan. 23, 2020

(54) MODULATION OF GUT MICROBIOTA IN HUNTINGTON'S DISEASE AND RETT SYNDROME

(71) Applicant: California Institute of Technology, Pasadena, CA (US)

(72) Inventors: Ali Khoshnan, Pasadena, CA (US); Sarkis K. Mazmanian, Pasadena, CA (US)

(21) Appl. No.: 16/483,720

(22) PCT Filed: Feb. 6, 2018
(86) PCT No.: PCT/US2018/017116

§ 371 Date: Aug. 5, 2019

Related U.S. Application Data
(60) Provisional application No. 62/455,706, filed on Feb. 7, 2017.

(51) Int. Cl. A61K 35/74; C12N 1/20; C12Q 1/689; C12Q 1/6883; A61P 25/22; A61P 25/14; A61K 35/745; A61K 35/747; A61K 35/744; A61K 31/437

(52) U.S. Cl. CPC A61K 35/74; C12N 1/20; C12Q 1/689; C12Q 1/6883; A61P 25/22; A61K 2035/115; A61K 35/745; A61K 35/747; A61K 35/744; A61K 31/437; C12Q 2600/158; A61P 25/14

(57) ABSTRACT

Some embodiments herein related to methods, composition, and/or uses for reducing the likelihood of, delaying the onset of, or ameliorating one or more symptoms associated with Rett syndrome or Huntington's disease. Without being limited by theory, it is contemplated that modulation of gut microbiota can affect symptoms of Rett syndrome and Huntington's disease. Compositions or product combinations comprising, consisting essentially of, or consisting of bacteria and/or antibiotics can be administered to subject in need thereof so as to reduce the likelihood of, delay the onset of, or ameliorate the symptoms.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This Application claims the benefit of US Provisional Application 62/455,706, filed Feb. 7, 2017, which is hereby incorporated by reference in its entirety.

### STATEMENT REGARDING FEDERALLY SPONSORED R&D

This invention was made with government support under NS074374 awarded by that National Institutes of Health. The government has certain rights in the invention.

### BACKGROUND

### Field

Some embodiments herein relate to methods and compositions for detecting, preventing, delaying the onset, and/or ameliorating symptoms associated with Huntington's disease and/or Rett Syndrome. The methods and compositions can comprise bacteria and/or antibiotics.

Huntington's disease (HD) is a devastating genetically inherited neurodegenerative disorder caused by expansion of a CAG repeat, which translates into a stretch of polyglutamine (polyQ) in the exon-1 of huntingtin (HTT) protein. The exon-1 of mutant HIT, which is produced proteolytically and by altered splicing of mRNA, is amyloidogenic, neurotoxic, and a major determinant of HD pathology (Bates et al, 2015). HD patients develop debilitating motor, psychiatric, and cognitive symptoms (Paulson, 2011). Environmental factors including inflammation may influence the pathogenesis of HD. Pre-manifest HD subjects have activated microglia and elevated levels of pro-inflammatory cytokines including IL-1β, IL-6, and TNF-α in the circulation and in the cerebrospinal fluid several years before the onset of motor symptoms (Bjorkqvist et al., 2008, Tai et at, 2007).

Rett syndrome is an X-linked autism-spectrum disorder (ASD) caused by mutations in the methyl-CpG binding protein 2 (MeCP2). Girls with Rett syndrome develop microcephaly, mental retardation, stereotypies, anxiety, breathing and speech problems, seizures, scoliosis, and Parkinson's disease features. MeCP2 mutations in males are lethal and those born usually die within a year. MeCP2 is also implicated in a subset of autistic patients, juvenile-onset schizophrenia, and MeCP2 duplication disorder (Lombardi et al., 2015).

### SUMMARY

Some embodiments include a composition or product combination comprising isolated bacteria that comprise at least two of: Actinobacteria bacteria, Tenericutes bacteria, or Bacteroides bacteria. In some embodiments, the composition or product combination comprises isolated Actinobacteria bacteria and isolated Bacteroides bacteria. In some embodiments, the composition or product combination comprises isolated Actinobacteria bacteria, in which the Actinobacteria bacteriacomprises Bifidobacteria. In some embodiments, the composition or product combination comprises isolated Bacteroides bacteria, and the Bacteroides bacteria are selected from the group consisting of: B. fragilis, B. ovatus, and B. thetaiotaomicron, or a combination of two or more of the listed bacteria. In some embodiments, the composition or product combination comprises Actinobacteria bacteria. Tenericutes bacteria, and Bactemides bacteria. In sonic embodiments, the composition or product combination comprises the isolated bacteria comprise bacteria that map to an OTU that maps to a bacterium selected from the group consisting of Mesoplasma entomophilum, Lactobacillus taiwanensis Pediococcus argentinicus, Bifidobacterium choerinum. In some embodiments, the bacteria map to an OTU when the bacteria comprise a 16S rRNA sequence of at least 100 nucleotides that is least 97% identical to a reference 16S rRNA sequence of the OUT, for example at least 97%, 98%, or 99%. In some embodiments, the composition or product combination comprises no more than 10⁶ cfu of Firmicutes bacteria, for example no more than 10⁵ cfu, 10⁴ cfu, 10³ cfu, 10² cfu, or 10 cfu. In some embodiments, the composition or product combination comprises a pharmaceutically acceptable excipient. In some embodiments, the composition or product combination comprises an antibiotic. In some embodiments, the composition or product combination comprises an antibiotic that is a rifamycin. In some embodiments, the antibiotic comprises, consists essentially of, or consists of rifaximin. In some embodiments, the antibiotic is in a separate composition that is separate from the isolated bacteria. In some embodiments, the isolated bacteria are in a single composition. In some embodiments, the isolated bacteria are in separate compositions from each other. In some embodiments, any of the compositions or product combinations as described herein is for use in reducing the likelihood of, delaying the onset of, or ameliorating one or more symptoms associated with Rett syndrome or Huntington's disease.

Some embodiments include a composition or product combination comprising an antibiotic, and an isolated bacteria selected from the group consisting of: Actinobacteria bacteria, Tenericutes bacteria, and Bacteroides bacteria. In some embodiments, the composition or product combination comprises the isolated Actinobacteria bacteria, and the Actinobacteria bacteria comprises Bifidobacteria. In some embodiments, the composition or product combination comprises the composition comprises no more than 10⁶ cfu of Firmicutes bacteria, for example no more than 10⁵ cfu, 10⁴ cfu, 10³ cfu, 10² cfu, or 10 cfu. In some embodiments, the antibiotic of the composition or product combination is a rifarnycin. In some embodiments, the antibiotic of the composition or product combination comprises, consists essentially of, or consists of rifaximin. In some embodiments, the antibiotic is in a separate composition that is separate from the isolated bacteria. In some embodiments, the composition or product combination further comprises a pharmaceutically acceptable excipient. In some embodiments, any of the compositions or product combinations as described herein is for use in reducing the likelihood of, delaying the onset of, or ameliorating one or more symptoms associated with Rett syndrome or Huntington's disease.

Some embodiments include a method of reducing the likelihood of, delaying the onset of, or ameliorating one or more symptoms associated with Rett syndrome or Huntington's disease in a subject in need thereof, the method comprising administering to the subject a composition or product combination comprising one or more bacteria, selected from the group consisting of: Actinobacteria bacteria, Tenericutes bacteria, and Bacteroides bacteria, or a combination of the listed bacteria. In some embodiments, the method further comprises selecting said subject as being within a class of subjects that should receive a composition for reducing the likelihood of, delaying the onset of, or ameliorating one or more symptoms associated with Rett syndrome or Huntington's disease. In some embodiments, the method reduces the likelihood of, delays the onset of, or ameliorates the one or more symptoms associated with Huntington's disease, and wherein Bacteroides bacteria are administered to the subject. In some embodiments, the method reduces the likelihood of, delays the onset of, or ameliorates the one or more symptoms associated with Huntington's disease, and wherein the Actinobacteria bacteria are administered to the subject. In some embodiments, the method reduces the likelihood of, delays the onset of, or ameliorates the one or more symptoms associated with Rett syndrome, and wherein the Actinobacteria bacteria and Bacteroides bacteria are administered to the subject. In some embodiments, the Bacteroides bacteria are administered to the subject simultaneously or separately. In some embodiments, the Actinobacteria bacteria and the Tenericutes bacteria are administered to the subject simultaneously or separately. In some embodiments, the composition or product combination the Actinobacteria bacteria comprise Bifidobacteria. In some embodiments, the composition or product combination the Bacteroides bacteria are selected from the group consisting of: B. fragilis, B. ovatus, and B. thetaiotaomicron. In some embodiments, the bacteria comprises bacteria that map to an OTU that maps to a bacterium selected from the group consisting of Mesoplasma entomophilum, Lactobacillus taiwanensis, Pediococcus argentinicus, Bifidobacterium choerinum. In some embodiments, a bacteria maps to au OTU when the bacteria comprise a 16S rRNA sequence of at least 100 nucleotides that is least 97% identical to a reference 16S rRNA sequence of the OUT, for example at least 97%, 98%, or 99%. In some embodiments, no more than 10⁴ cfu of Firmicutes bacteria is administered to the subject, for example no more than 10⁵ cfu, 10⁴ cfu, 10³ cfu, 10² cfu, or 10 cfu. In sotne embodiments, the antibiotic is a rifamycin.

Some embodiments include a method of reducing the likelihood of, delaying the onset of, or ameliorating one or more symptoms associated with Rett syndrome or Huntington's disease in a subject in need thereof. The method can comprise administering an antibiotic to the subject. In some embodiments, the method further comprises selecting said subject as one being within a class of subjects that should receive a composition for reducing the likelihood of, delaying the onset of, or ameliorating one or more symptoms associated with Rett syndrome or Huntington's disease. In some embodiments, the antibiotic is a rifamycin. In some embodiments, the antibiotic comprises, consists essentially of, or consists of rifaximin. In some embodiments, the one or more symptoms associated with Rett syndrome are reduced in likelihood, delayed in onset, or ameliorated. In some embodiments, the one or more symptoms associated with Huntington's disease are reduced in likelihood, delayed in onset, or ameliorated. In some embodiments, administering the antibiotic reduces a quantity of gut bacteria in the subject by at least 95%, for example at least 95%, 96%, 97%, 98%, 99%, including ranges between any two of the listed values. In some embodiments, the method further comprises administering a composition or product combination comprising isolated bacteria selected from the group consisting of: Actinobacteria bacteria, Tenericutes bacteria, and Bacteroides bacteria, or a combination of the liked bacteria to the subject. In some embodiments, the isolated bacteria comprise bacteria that map to an OTU that maps to a bacterium selected from the group consisting of Mesoplasma entomophilum, Lactobacillus taiwanensis, Pediococcus argentinicus, Bifidobacterium choerinum. In some embodiments, a bacteria maps to an OTU when the bacteria comprise a 16S rRNA sequence of at least 100 nucleotides that is least 97% identical to a reference 165 rRNA sequence of the OUT, for example at least 97%, 98%, or 99%. In some embodiments, the Actinobacteria bacteria and Bacteroides bacteria are administered to the subject. In some embodiments, the Actinobacteria bacteria are administered to the subject, and the Actinobacteria bacteria comprises Bifidobateria. In some embodiments, the Bacteroides bacteria are administered to the subject, and the Bacteroides bacteria are selected from the group consisting of: B. fragilis, B. ovatus, and B. thetaiotaomicron, or a combination of two or more of the listed bacteria. In some embodiments, the isolated bacteria are administered simultaneously with the antibiotic. In some embodiments, the isolated bacteria are administered at a different time than the antibiotic. In some embodiments, (a) the antibiotic is administered prior to the bacteria; or (b) the bacteria is administered prior to the antibiotic. In some embodiments, no more than 10⁴ cfu of Firmicutes bacteria is administered to the subject for example no more than 10⁵ cfu, 10⁴ cfu, 10³ cfu, 10² cfu, or 10 cfu.

Some embodiments include a method of determining a profile of a sample of a subject. The method comprising detecting at least one of: (a) a presence and/or level of a gut bacterium selected from the group consisting of: Tenericutes, Actinobacteria, and Firmicutes, or a combination of two or more of the listed bacteria; (b) a serum level of a neurotransmitter selected from the group consisting of Choline, 5-HT, Tyrosine, Dopamine, and Epinepherine, or two or more of the listed neurotransmitters, or (c) an expression level of a cholinergic gene selected from the group consisting of: Chrna2, Chrna7, Chrb4, Chrm1, Slc5a7, Chat, Ache, and Slc18a3, or two or more of the listed genes. The profile can comprise the detected presence and/or levels of (a), (b), (c), (a) and (b), (a) and (c), (b) and (c), or (a) and (b) and (c). In some embodiments, determining the profile comprises determining (a), wherein the sample comprises gut and/or feces material of the subject, and wherein a presence or elevated risk of Rett syndrome is indicated by lower levels of Tenericutes or Actinobacteria, or increased levels of Firmicutes, relative to levels present in a non-Rett control sample. In some embodiments, determining the profile comprises determining (b), wherein the sample comprises serum of the subject, and a presence or elevated risk of Rett syndrome is indicated by: higher levels of Choline, Tyrosine, and/or Dopamine compared to a control; and/or lower levels of 5H-T and/or Epinepherine,relative to levels present in a non-Rett control sample. In some embodiments, determining the profile comprises determining (c), and the sample comprises nucleic acids of the subject, and a presence or elevated risk of Rett syndrome is indicated by lower expression levels of Chrna2, Chrna7, Chrh4, Chrm1, Slc5a7, Chat, Ache, Slc18a3, or two or more of the listed genes, relative to levels present in a non-Rett control sample. In some embodiments, the Actinobacteria comprise Bifidobacteria.

### BRIEF DESCRIPTION OF THE DRAWINGS

FIGS. 1A-D. FIG. 1A is a series of images depicting immunohistochemistry of the aggregated mHDx1 in the Drosophila third instar larvae −/30 rifaximin (Rif) of some embodiments. FIG. IB is an image of a Western blot (WB) of different batch pooled larvae examined for MHDx1 aggregation. FIG. 1C is a series of images depicting immunohistochemistry similar to FIG. 1A except that adult flies were used in FIG. 1C. FIG. 1D depicts a WB similar to FIG. 1B, except that adult flies were used in FIG. 1D. The Gal4 induction system with mHDx1 (120Q) downstream of UAS (upstream activation sequence) and the gal4 transactivator downstream of Drosophila neuronal-specific promoter Elav was used to induce the expression of mHDx1 in the nervous system of offspring. In FIG. 1C, the central brain region is shown.

FIGS. 2A-D are a series of graphs depicting effects of feeding bacteria to HD flies in accordance with some embodiments herein. FIG. 2A depicts feeding E. coli to HD flies accelerates the development of motor symptoms. FIG. 2B depicts feeding Lactobacillus rhamnosus (LGG) to HD flies does not affect motor symptoms. FIG. 2C depicts antimicrobial peptide mRNAs are elevated in HD flies. FIG. 2D depicts elimination of gut microbes by Penicillin-Streptomycin ameliorates motor defects in HD flies.

FIGS. 3A-D are a series of images depicting effects of bacteria on HD flies in accordance with some embodiments herein. FIG. 3A shows images depicting Immunohistochemistry of Drosophila gut with an anti-Curli antibody recognizing oligomers (white arrow). FIG. 3B depicts staining of adult 586 HD flies with an antibody recognizing the aggregates only in those colonized with Curli+E. coli. FIG. 3C is an image depicting western blot results (WB) of pooled samples from similar flies as in B. FIG. 3D depicts a climbing assay showing the immobility of flies colonized with Curli+E. coli. The Gal4 system with 586 AA (120Q) N-terminal fragment of mutant HTT downstream of UAS and the gal4 transactivator downstream of neuronal-specific promoter Elav was used to induce the expression in the nervous system of offspring.

FIGS. 4A-B are a set of images depicting brain section of WT and HD Drosophila in accordance with some embodiments herein. Shown are EM analysis of brain sections of WT (control)(FIG. 4A) and HD (FIG. 4B) with an oligomeric specific antibody (PHP2). Immune gold labeling of the oligomers is shown as black dots.

FIGS. 5A-D are a series of images and graphs showing characteristics of Rett mice with and without rifaximin treatment in accordance with some embodiments herein. FIG. 5A depicts nest building ability of Rett mice +/− rifaximin. FIG. 58 is a graph depicting that rifaximin-treated mice make more attempts at climbing a wired-mesh cylinder within a 2 min time allowed. FIG. 5C is a graph depicting that testing muscle strength by wired mesh hanging assay. FIG. 5D is a graph depicting hindlimb clasping of Rett mice in the presence and absence rifaximin. N=6. Scoring and procedures were as described in Southwell et al., 2009.

FIGS. 6A-B are a series of images and graphs depicting the results of doublecortin staining of Rett mice of some embodiments. FIG. 6A depicts doublecortin staining of bilateral hippocampus in vehicle-treated (top panels) and rifaximin-treated Rett mice (bottom panels). FIG. 6B shows quantification of doublecortin positive cells in the dentate gyrus (DG) of bilateral hippocampi (2layers) from 4 vehicle-treated and 6 rifaximin-treated animals

FIG. 7 is an image depicting the results of semi-quantitative PCR of Rett mice of some embodiments. Semi-quantitative PCR shows the absence of Tenericutes (Ten) in male Rett mice and elevation upon treatment with rifaximin(Rif) (bottom panels). Top panels are Eubacteria amplification used as a positive control.

FIGS. 8A-B are a series of images showing immunohistochemistry for MeCP2 in accordance with some embodiments herein. FIG. 8A depicts immunohistochemistry of the ileum portion of the small intestine showing MeCP2 expression in the intestinal epithelium (left panels). Arrow at the bottom of right panel points to the crypt cells. FIG. 8B depicts staining for MeCP2 in lamina propria cells as well myenteric plexus in the enteric nervous system (top panel). Bottom panel is a similar section from a Rett mouse (T158A).

FIGS. 9A-F are a series of images and graphs showing analysis of intestines of WT and Rett mice of some embodiments. FIGS. 9A and 9B are graphs depicting quantification of small and large intestines length in WT and T158A Rett mice. Each dot represents a mouse. FIG. 9C is an image of representative GI tracts of WT and Rett mice. FIGS. 9D and 9E are graphs depicting changes in the gut length are minimal in young animals. FIG. 9F is a pair of representative sections of the ileum of WT (left) and T158A (right) Rett mice stained with an anti-lysozyme antibody to identify paneth cells (arrows) in the crypts at the base of villi. DAPI stain as used to mark all cells and reveal the structure of the villi.

FIG. 10 is an image depicting Bromodeoxyuridine (BrdU) incorporation in the intestinal epithelium of WT (left) and Rett (right) mice of some embodiments. 4 weeks old mice were injected with BrdU. Tissue was harvested 24 hr post-injection, processed, and stained with antibodies recognizing BrdU (which appear as the bright foci that are not marked with arrows in FIG. 10) and ki67 (arrows) a marker of proliferating cells. Amplifying progenitor cells are labeled with BrdU.

FIGS. 11A-D are a series of graphs and images depicting analysis of bacterial phyla in fecal pellets of WT and Rett mice. FIGS. 11.E and 11B are graphs showing percentage of major bacterial phyla present in the fecal pellets of WT (FIG. 11A) and T158A (FIG. 11B) mice determined by 16S RNA sequencing. Those indicated in red are different between the two groups. N=6 for each group, raised in different cages. FIG. 11C is an image showing semi-quantitative PCR showing the absence of Bifidobacteria in the male Rett mice. FIG. 11D is an image indicating reduction of Bifidobacteria in the 4 months-old female Rett mice.

FIGS. 12A-D are a series of graphs and images depicting characteristics of WT and Rett mice of some embodiments. Rett mice have transparent small intestine, elevated levels of lipopolysaccharide (LPS) in the circulation (See FIGS. 12A-D), and accumulate excess abdominal fat (See FIGS. 12C-D). FIG. 12E is an image showing representative GF WT and Rett mice lacking abdominal fat. FIG. 12F is an image showing that the gut length in the Rett mice is similar to WT. For FIGS. 12B and 12D, N=6 for each group.

FIG. 13 is a series of images showing immunostaining of macrophages in the ileum portion of the small intestine of WT are Rett mice of some embodiments. The immunostaining was performed with antibodies to macrophage markers Iba-1 (arrowheads) and F4/80 (arrows). Each row is representative from one mouse in each group.

FIGS. 14A-F are a series of images and graphs depicting the results of treating Rett mice with an antibiotic cocktail in accordance with some embodiments herein. FIG. 14A is a graph depicting that treatment of Rett mice with an antibiotic cocktail (ABX) improves small intestine (SI) length, whereas rifaximin (Rif) promotes colon length (FIGS. 14B-C). In FIG. 14A, each point represents a mouse. N=6 each in FIG. 14C. FIG. 14D presents the extent of obesity in heterozygous females. FIGS. 14E-F show weight gain and the accumulation of abdominal fat is prevented by treatment of female Rett mice with rifaximin . N=6 for FIGS. 14E and 14F. Rifaximin has minimal effect on the weight of WT mice.

FIGS. 15A-B are a series of images depicting staining of brain sections from the cortical region of Rett mice treated with vehicle or rifaximin in accordance with some embodiments herein. FIG. 15A depicts staining with markers of activated astrocyte GFAP (center column) and GS (right column); Overlay of GFAP and GS is shown in left column. FIG. 15B shows staining with the microglial markers Iba-1 and CD11 in the hippocampal region. The Iba-1 and CD11 generally colocalized throughout the hippocampal region, but Iba-1 was expressed at higher levels in the vehicle-treated group (see, e.g, arrow), and lower levels in the rifaximin-treated group.

FIGS. 16A-D are a series of graphs and images showing behaviors of Rett mice in the presence or absence of rifaximin in accordance with some embodiments herein. FIG. 16A shows nest building ability of Rett mice in the presence or absence of rifaximin. FIG. 16B shows Rifaximin-treated mice make more attempts at climbing a wired-mesh cylinder within a 2 min time allowed. FIG. 16C shows testing muscle strength by wired mesh hanging assay. FIG. 160 shows hind limb clasping of Rett mice in the presence and absence rifaximin. N=6. Scoring and procedures were as described in Southwell et al., 2009.

FIGS. 17A-B are a series of microscope images showing doublecortin staining of bilateral hippocampus of WT and Rett (T158A) mice of some embodiments. FIG. 17A is a series of images depicting doublecortin staining of bilateral hippocampus in WT (top panels) and Rett (T158A) mice (bottom panels). FIG. 17B is a series of images showing similar staining of GF WT (top panels) and TI58A male mice.

FIGS. 18A-C are a series of graphs and images depicting effects of microbiota on neurogenesis in Rett mice in accordance with some embodiments herein. FIG. 18A is a series of images depicting doublecortin staining of bilateral hippocampus in vehicle-treated (top panels) and rifaximin-treated Rett mice (bottm panels). FIG. 18B is a graph showing quantification of doublecortin positive cells in the dentate gyrus of bilateral hippocampi (2 layers) from 4 vehicle-treated and 6 rifaximin-treated animals, FIG. 18C is a series of images showing ki67 positive cells (arrows) in the SVZ (subventricular zone) of vehicle (top) and rifaximin-treated Rett mice. Boxed areas highlight regions with dramatic changes (considerably more ki67-positive cells are observed in the rifaximin-treated mose). NeuN staining for neurons (NeuN) is observed as bright foci throughout.

FIG. 19 is an image showing the results of semi-quantitative PCR in WT and Rett mice in accordance with some embodiments herein. Semi-quantitative PCR shows the absence of Tenericutes (Ten) in male Rett mice and elevation upon treatment with rifaximin (Rif) (bottom panels), Top panels are Eubacteria amplification used as a positive control.

FIG. 20A is a graph depicting expression of cholinergic genes in Rett mice of some embodiments. It is shown that Rett mice have reduced expression of cholinergic genes.

FIG. 20B is a graph depicting neurotransmitters levels in the serum of Rett mice of some embodiments.

### DETAILED DESCRIPTION

Described herein are methods, compositions, and product combinations for reducing the likelihood of, delaying the onset of, or ameliorating one or more symptoms associated with Huntington's disease (also referred to herein as “HD”) or Rett syndrome (also referred to herein as “Rett”). It is observed herein that in a Drosophila model of HD, altering the microbial flora can exacerbate the symptoms of HD, while eliminating gut microbes using an antibiotic improves the symptoms of HD (See, e.g., Example 2). As such, in some embodiments, methods, compositions, and product combinations comprising antibiotics and/or bacteria are provided for reducing the likelihood of, delaying the onset of, or ameliorating one or more symptoms associated with HD. It is further observed that in a mouse model of Rett, the gut microbiota is altered at the species and/or phyla levels compared to wild-type mice, as is gut morphology (See, e.g., Examples 9-10), On the other hand, eliminating gut microbes in this mouse model using an antibiotic ameliorates physiological and behavioral symptoms in the Rett mice (See, e.g., Examples 14-18). Accordingly, in sonic embodiments herein, methods, uses, compositions, and/or product combinations comprising antibiotics and/or bacteria are provided for reducing the likelihood of, delaying the onset of, or ameliorating one or more symptoms associated with Rett syndrome. Also provided are methods and compositions for determining a profile of a sample of a subject, which can be useful, for example, to determine an elevated risk and/or presence of Rett syndrome.

HD patients display gastrointestinal (GI) complications. However, studies on the onset and severity of GI defects in HD are scarce (Andrich et al., 2009). Recent studies demonstrate that the GI tract and its resident microbes (which may be referred to herein as the “microbiota,” and their collective genomes as the “microbiome”) influence neurodevelopment, neurotrophin and neurotransmitter production, and behavior (Dinan et al., 2016, Sherwin et al., 2016). Without being limited by theory, it is contemplated herein that the homeostasis of intestinal microbiota may contribute to the pathogenesis of HD.

Impaired brain circuits are the underlying cause of Rett syndrome but defects in other organs and metabolic aberrations may play a role. Potential modifiers of Rett pathogenesis include abnormalities in the immune system and inflammatory pathways, cholesterol and lipid metabolism, and insulin-like growth factor 1 (IGF-1) and brain-derived neurotrophic factor (BDNF) signaling (Tropea et al., 2009, Buchovecky et al., 2013, Cronk et al., 2015, Lombardi et al., 2015). Rett patients display severe gastrointestinal (GI) complications (Motil et al., 2012). Recent studies demonstrate that the GI tract and its resident microbes influence neurodevelopment, neurotrophin and neurotransmitter production, and behavior (Sherwin et al., 2016). Without being limited by theory, it is contemplated herein that the GI pathology and changes in the homeostasis of intestinal microbiota may contribute to the pathogenesis of Rett syndrome.

### Compositions and Product Combinations

Compositions and/or product combinations comprising, consisting essentially of, or consisting of certain bacteria and/or antibiotics (e.g., rifamyacins, for example rifaximin; additional example antibiotics are described below) in accordance with various embodiments herein are useful for reducing the likelihood of, delaying the onset of, and/or ameliorating one or more symptoms associated with Huntington's disease or Rett syndrome. It noted that in some embodiments, the components of any of the noted compositions can be provided separately as “product combinations” in which the components are provided in two or more precursor compositions, which can either be combined to form the final composition (e.g., mix bacteria with another bacteria and/or one or more antibiotics to arrive at a final composition comprising a mixture of bacteria and/or antibiotic(s)) or used in conjunction to achieve an effect similar to the single composition (e.g., administer bacteria and one or more antibiotics to a subject simultaneously or sequentially). Accordingly, wherever a composition comprising two or more components is described herein, a corresponding “product combination,” which collectively contains the components of the composition is also expressly contemplated. It is further contemplated that any of the compositions and/or product combinations can be administered in use or a method for reducing the likelihood of, delaying the onset of, or ameliorating one or more symptoms associated with Huntington's disease (also referred to herein as “HD”) or Rett syndrome (also referred to herein as “Rett”) as described herein

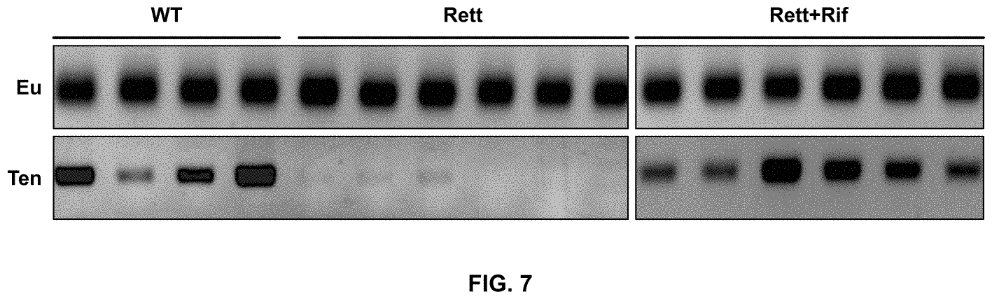

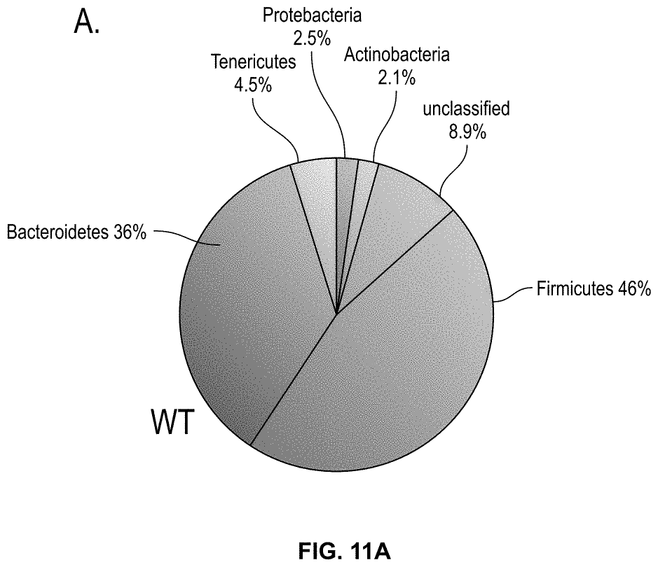

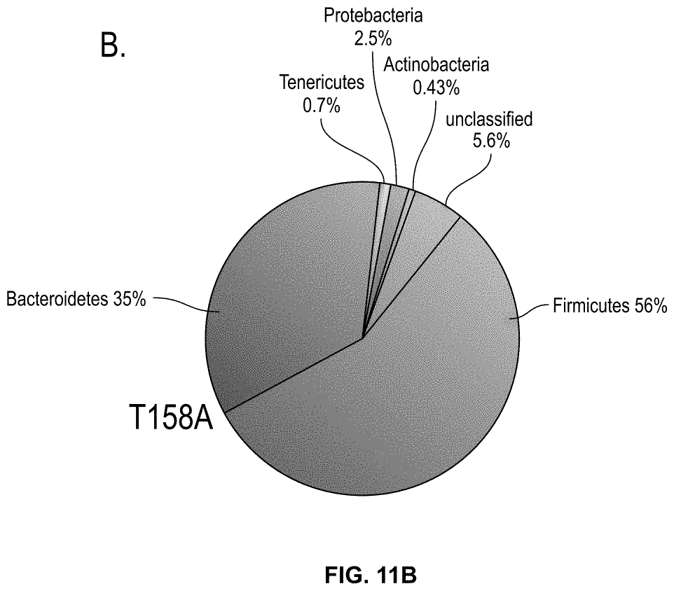

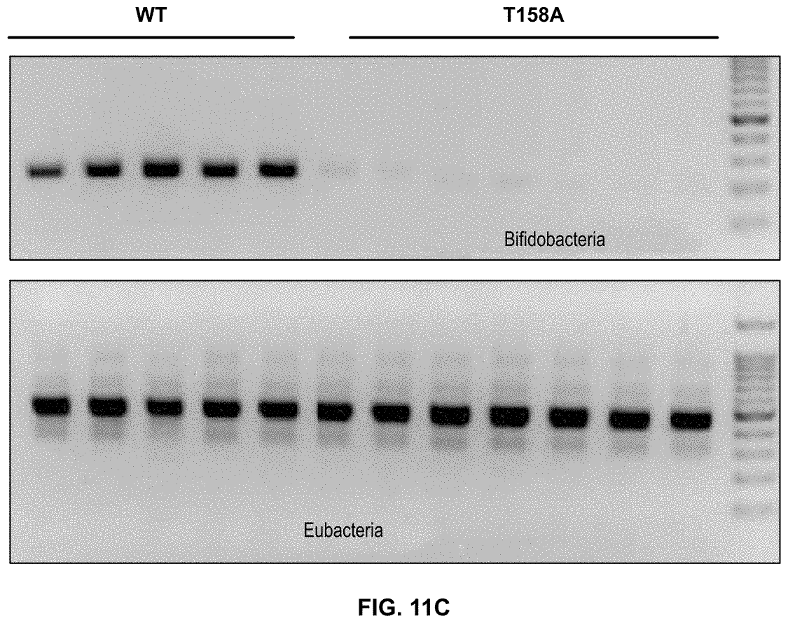

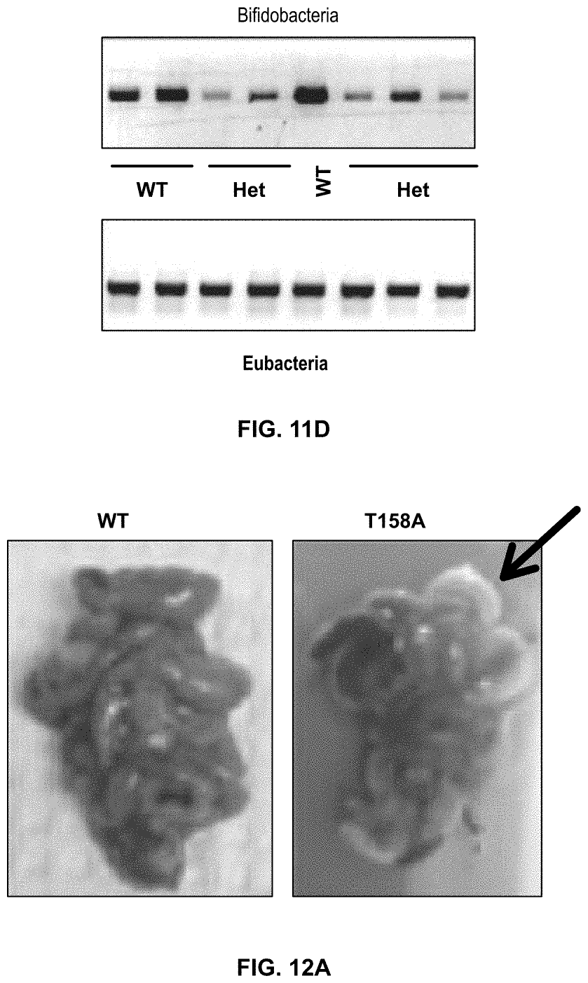

It is observed herein that modulating the gut microbiota by administering E. coli to a Drosophila HD model can exacerbate the motor symptoms of HD (See, e.g., Examples 2 and 3). On the other hand, treating the Drosophila HD model with the antibiotics penicillin-streptomycin (so as to reduce and/or eliminate gut bacteria) in accordance with some embodiments herein can reduce the accumulation of Huntingtin aggregates in the Central Nervous System (CNS) and ameliorate the motor symptoms of HD (See, e,g., Example 3). Accordingly, it is contemplated that compositions comprising bacteria and/or antibiotics in accordance with some embodiments herein are useful for reducing the likelihood of, delaying the onset of, and/or ameliorating one or more symptoms associated with HD. It is further observed that the gut microbiota differ between wild-type mice and a mouse genetic model of Rett syndrome (See Example 9). Particular phyla and species differ in fecal samples of the Rett model compared to wild-type mice, so that some species and/or phyla are overrepresented in the gut of Rett syndrome mice, while others are underrepresented (FIGS. 7, 11A-D). In particular, is reported herein that Rett mice have significantly less Tenericutes and Actinobacteria and significantly more Firmicutes bacteria than control (wild-type) mice (See Example 8 and FIGS. 11A-D). Bifidobacteria (a genus within Actinobacteria) are eliminated from the GI tract of Rett mice starting at ˜6 weeks of age (Example 8, FIG. 7). Bacteria that were differently expressed in Rett mice and wild-type (control) mice are show in Table 1, below. Moreover, treating Rett mice with an antibiotic comprising rifaximin in accordance with some embodiments herein reduced systemic inflammation, influenced morphology of the central nervous system included astrocytes and microglia, and ameliorated behavioral defects associated with the Rett syndrome model, such as defects in nest building, climbing, and muscle strength (See Examples 14-16). Accordingly, it is contemplated that compositions comprising bacteria and/or antibiotics in accordance with some embodiments herein are useful for treating the likelihood of, delaying the onset of, and/or ameliorating one or more symptoms associated with Rett syndrome.

TABLE 1
Bacteria that differed in Rett and wild-type mice

| | | Elevated or Decreased |
|---|---|---|
| | | in Rett compared to |
| Species | Phylum | wild-type (control) |
| Lactobacillus hayakitensis | Firmicutes | Elevated in Rett |
| Mesoplasma entomophilum | Tenericutes | Decreased in Rett |
| Lactobacillus taiwanensis | Firmicutes | Decreased in Rett |
| Pediococcus argentinicus | Firmicutes | Decreased in Rett |
| Lactobacillus intestinalis | Firmicutes | Elevated in Rett |
| Bifidobacterium choerinum | Actinobacteria | Decreased in Rett |

In some embodiments a composition or product combination comprises, consists essentially of, or consists of bacteria. In some embodiments, the composition or product combination comprises isolated bacteria that comprise at least two of: Actinobacteria bacteria (e.g., Bifidobacteria such as Bifidobacterium choerinum), Tenericutes bacteria (e.g., Mesoplasma bacteria such as Mesoplasma entomophilum), orBacteroides bacteria (e.g., B. fragilis, B. ovatus, and/or B. thetaiotaomicron). For example, in some embodiments, the composition or product combination comprises, consists essentially of, or consists of Actinobacteria and Bacteroides bacteria. In some embodiments, the composition or product combination comprises, consists essentially of, or consists of Bifidobacteria and Bacteroides. In some embodiments, the composition or product combination comprises, consists essentially of, or consists of Actinobacteria and Tenericutes bacteria. In some embodiments, the composition or product combination comprises, consists essentially of, or consists of Bifidobacteria and Tenericutes. In some embodiments, the composition or product combination comprises, consists essentially of, or consists of Tenericutes bacteria and Bacteroides bacteria. In some embodiments, the composition or product combination comprises, consists essentially of, or consists of Actinobacieria bacteria, Tenericutes bacteria, and Bacteroides bacteria. In some embodiments, the composition or product combination comprises, consists essentially of, or consists of Bifidobacteria bacteria, Tenericutes bacteria, and Bacteroides bacteria. In some embodiments, a composition or product combination as described herein comprises the Actinobacteria bacteria, and the Actinobacteria bacteria comprises Bifidobacteria. In some embodiments, a composition or product combination as described herein comprises the Bacteroides bacteria, and the Bacteroides bacteria are selected from the group consisting of: B. fragilis, B. ovatus, and B. thetaiotaomicron, or a combination of two or more of the listed bacteria (e.g., B. fragilis and B. ovatus; B. fragilis and B. thetaiotaomicron; B. ovatus, and B. thetaiotaomicron; or B. fragilis, B. ovatus, and B. thetaiotaomicron). In some embodiments, the composition or product combination as described herein comprises the Tenericutes bacteria, and the Tenericutes bacteria comprise, consist essentially of, or consist of Mesoplasma bacteria such as Mesoplasma entomophilum. In some embodiments, the bacteria of the composition or product combination are together in a single composition. In some embodiments, the bacteria of the product combination are in two or more separate compositions, which, optionally, can be mixed prior to, or at the time of use. In some embodiments, the separate compositions can be used (administered) separately. In some embodiments, the composition or product combination is for use in reducing the likelihood of, delaying the onset of, or ameliorating one or more symptoms associated with Rett syndrome or Huntington's disease. Optionally, the composition or product combination can be for use in a subject selected as being within a class of subjects that should receive the composition (or product combination) for reducing the likelihood of, delaying the onset of, or ameliorating one or more symptoms associated with Rett syndrome or Huntington's disease. For example, a profile of a sample of a subject as described herein can indicate a presence or elevated risk of Rett syndrome or Huntington's disease, and/or that the subject is amendable to treatment using a composition or product combination comprising bacteria and/or antibiotic as described herein. In some embodiments, the composition or product combination is for use in reducing the likelihood of, delaying the onset of, or ameliorating one or more symptoms associated with Rett syndrome. In some embodiments, the composition or product combination is for use in reducing the likelihood of, delaying the onset of, or ameliorating one or more symptoms associated with Huntington's disease.

In some embodiments, the composition or product combination comprises, consists essentially of, or consists of an antibiotic as described herein (e.g. such as rifamyacins, for example rifaximin; additional example antibiotics are noted below), and an isolated bacteria selected from the group consisting of: Actinobacteria bacteria (e.g., Bifidobacteria such as Bifidobacterium choerinum), Tenericutes bacteria (e.g., Mesoplasma bacteria such as Mesoplasma eniomophilum), and Bacteroides bacteria (e.g., B. fragilis, B. ovatus, and/or B. thetaiotaomicron), or a combination of two or more of the listed bacteria. In some embodiments, the composition or product combination comprises, consists essentially of the antibiotic (e.g., as rifamyacins, such as rifaximin; additional example antibiotics are noted below) and two or more bacteria, for example, Actinobacteria and Bacteroides bacteria; Actinobacteria and Tenericutes bacteria; Tenericutes bacteria, and Bacteroides bacteria; or Actinobacteria bacteria, Tenericutes bacteria, and Bacteroides bacteria. In some embodiments, the composition or product combination comprises the Actinobacteria bacteria, and the Actinobacteria bacteria comprise, consist essentially of, or consist of Bifidobacteria. In some embodiments, the composition or product combination comprises, consists essentially of, or consists of the antibiotic and Bifidobacteria and Bacteroides (e.g., B. fragilis, B. ovatus, and/or B. thetaiomomicron). Optionally, the antibiotic is rifaximin. In the product combination of some embodiments, the antibiotic is in a separate composition that is separate from the isolated bacteria.

Without being limited by theory, it is contemplated that combinations of bacteria as described herein can have a synergistic probiotic effect, for example to ameliorate, delay the onset of, or decrease the likelihood of symptoms of Rett syndrome and/or HD. It has been reported that two species of Bifidobacteria (Bifidobacterium longum and Bifidobacterium animalis) produce exopolysaccharides (EPS) that are used by B. fragilis as fermentable substrates (Rios-Covian et al., 2016, BMC Microbiology 16: 1:50, which is hereby incorporated by reference in its entirety).

In some embodiments, the composition or product combination is for use in reducing the likelihood of, delaying the onset of, or ameliorating one or more symptoms associated with Rett syndrome or Huntington's disease. The composition or product combination can be for use in a subject selected as being within a class of subjects that should receive the composition (or product combination) for reducing the likelihood of, delaying the onset of, or ameliorating one or more symptoms associated with Rett syndrome or Huntington's disease. For example, a profile of a sample of a subject as described herein can indicate a presence or elevated risk of Rett syndrome or Huntington's disease. For example, a profile of a sample of a subject as described herein can indicate a presence or elevated risk of Rett syndrome or Huntington's disease, and/or that the subject is amendable to treatment using a composition or product combination comprising bacteria and/or antibiotic as described herein. In some embodiments, the composition or product combination is for use in reducing the likelihood of, delaying the onset of, or ameliorating one or more symptoms associated with Rett syndrome. In some embodiments, the composition or product combination is for use in reducing the likelihood of, delaying the onset of, or ameliorating one or more symptoms associated with Huntington's disease.

In some embodiments, any of the compositions or product combinations as described herein comprise, consist essentially of, or consist of one or more bacteria that are underexpressed in Rett syndrome as shown in Table 1 (e.g., Mesoplasma entomophilum, Lactobacillus taiwanensis, Pediococcus argentinicus, and Bifidobacterium choerinum). In some embodiments, any of the compositions or product combinations as described herein comprise, consist essentially of, or consist of one or more bacteria that maps to an OTU that maps to a bacterium selected from the group consisting of Mesoplasma entomophilum, Lactobacillus taiwaraensis Pediococcus argentinicus, and Bifidobacterium choerinum. For example, the composition can comprise at least 1, 2, 3, 4, 5, 6, 7, 8, 9, or 10 bacteria ranges between any two of the listed values, for example 1-3, 1-5, 1-10, 2-3, 2-5, 2-10, 3-5, or 3-10) that each map to an OTU that maps to any of Mesoplasma entomophilum, Lactobacillus taiwanensis, Pediococcus argentinicus, Bifidobacterium choerinum (it noted that the different bacteria of the composition or product combination can map to OTUs that are the same or different).

In some embodiments, bacteria map to an OTU when the bacteria comprise a 16S rRNA sequence of at least 100 nucleotides that has at least 95%, 96%, 97%, 98%, or 99% identity to a reference 16S rRNA sequence of the OTU. In some embodiments, bacteria map to an OTU when the bacteria comprise a 16S rRNA sequence of at least 100 nucleotides that has at least 97% identity to a reference 16S rRNA sequence of the OTU.

In some embodiments, the bacteria of any of the compositions or product combinations, uses, or methods described herein are isolated. As used herein, “isolated” bacteria, including and variations of this root term, has its customary and ordinary meaning as understood by one of skill in the art in view of this disclosure. It refers to bacteria that are not in their endogenous growth environment and/or are not in the composition in which they grow endogenously. It is not required that an “isolated” bacteria be in a composition free of all other substances. By way of example, bacteria cultivated outside of their endogenous growth environment are an example of isolated bacteria in the context of this Application. By way of example, gut bacteria that are outside of the gut and apart from a raw fecal sample are an example of “isolated” bacteria. On the other hand, it is understood herein that gut bacteria in a raw fecal or gut contents sample are not an example of “isolated” bacteria in the context of this Application. Accordingly in some embodiments, isolated bacteria (such as Actinobacteria bacteria, Tenericutes bacteria, and/or Bacteroides bacteria) are not part of a fecal sample, and are not part of a gut contents sample.

in some embodiments, the composition, product combination, use, and/or method comprises an amount of bacteria sufficient to establish a colony (e.g., a colony that persists for at least 1, 2, 3, 4 or more weeks post-inoculation) in the gut of a human subject when administered in a standard manner for microbiome transplant, probiotic treatment or equivalent procedures. Such an amount of bacteria may be referred to herein as an “inoculum.” In some embodiments, the amount of bacteria in the composition, product combination, use, or method includes at least 10⁴ colony forming units (cfu), for example at least 10⁴, 10⁵, 10⁶, 10⁷, 10⁸, 10⁹, 10¹⁰, 10¹¹, 10¹², or 10¹³ cfu, including ranges between any of the listed values, for example 10⁴-10⁸ cfu, 10⁴-10⁹ cfu, 10⁴-10¹⁰ cfu, 10⁴-10¹¹ cfu, 10⁴-10¹² cfu, 10⁴-10¹² cfu, 10⁵-10⁸ cfu, 10⁵-10⁹ cfu, 10⁵-10¹⁰ cfu, 10⁵-10¹¹ cfu, 10⁵-10¹² cfu, 10⁵-10¹² cfu, 10⁶-10⁸ cfu, 10⁶-10⁹ cfu, 10⁶-10¹⁰ cfu, 10⁶-10¹¹ cfu, 10⁶-10¹² cfu, 10⁶-10¹² cfu, 10⁷-10⁸ cfu, 10⁷-10⁹ cfu, 10⁷-10¹⁰ cfu, 10⁷-10¹¹ cfu, 10⁷-10¹² cfu, 10⁷-10¹² cfu, −10⁹ cfu, 10⁸-10¹⁰ cfu, 10⁸-10¹¹ cfu, 10⁸-10¹² cfu, or 10⁸-10¹² cfu. In some embodiments, the composition, product combination, use, and/or method comprises a log phase (at 37° C.) of bacteria for administration to the subject. In some embodiments, the composition, product combination, use, and/or method comprises a stationary phase (at 37° C.) of bacteria for administration to the subject. In some embodiments, the bacteria of the composition, product combination, use, and/or method are isolated bacteria.

It is noted that Firmicutes bacteria (e.g., Lactobacillus, such as Lactobacillus hayakitensis and Lactobacillus intestinalis) are significantly increased in fecal samples of Rett syndrome mice. Accordingly, in some embodiments, any of the compositions or product combinations, uses, and/or methods described herein is free or substantially free of Firmicutes bacteria (e.g., Lactobacillus, such as Lactobacillus hayakitensis and Lactobacillus intestinalis). As used herein, “substantially free” and variations of this root term has its customary and ordinary meaning as understood by one of skill in the art in view of this disclosure. It refers to a composition and/or product combination (which may be for a use or a method as described herein) having no more than trace amounts of a substance (e.g., a bacteria such as Firmicutes), and/or the amount or presence of the substance having no appreciable effect (e.g., behavioral effect) on the subject. For example, in some embodiments, a composition and/or product combination substantially free of a bacteria comprises no more than about 10⁶ cfu of that bacteria, for example no more than 10⁶ cfu, 10⁵ cfu, 10⁴ cfu, 10³ cfu, 10² cfu, or 10 cfu. In some embodiments, a composition and/or product combination substantially free of a bacteria comprises no more than about 10⁶ cfu of that bacteria, for example no more than 10⁶ cfu, 10⁵ cfu, 10⁴ cfu, 10³ cfu, 10² cfu, or 10 cfu. In some embodiments, a composition and/or product combination substantially free of a bacterium comprises no more than about 10⁴ cfu of that bacterium. Accordingly, a composition and/or product combination substantially free of Firmicutes in accordance with compositions, methods, and uses of some embodiments herein, may comprise Firmicutes in trace amounts, and/or the amount or presence Firmicutes has no appreciable behavioral effect on the subject. By way of example, in some embodiments, a composition or product combination is substantially free of Firmicutes when it comprises no more than 10⁶ cfu, 10⁵ cfu, 10⁴ cfu, 10³ cfu, 10² cfu, or 10 cfu of Firmicutes. Accordingly, in some embodiments, any of the compositions or product combinations described herein comprises no more than 10⁶ cfu, 10⁵ cfu, 10⁴ cfu, 10³ cfu, 10¹ cfu, or 10 cfu of Firmicutes. In some embodiments, any of the compositions or product combinations described herein comprises no more than 10⁶ du, 10³ cfu, 10⁴ cfu, 10³ cfu, 10² cfu, or 10 cfu of Lactobacillus (a genus of Firmicutes), such as Lactobacillus hayakitensis and/or Lactobacillus intestinalis.

In some embodiments, any of the compositions and/or product combinations described herein (including those for uses and/or methods as described herein) comprises nutrients or media in which the bacteria were cultured or additional nutrients that increase the likelihood of successfully establishing the colony.

In some embodiments, any of the compositions and/or product combinations described herein (including those for uses and/or methods as described herein) comprises a pharmaceutically acceptable carrier or excipient. “Pharmaceutically acceptable” carriers have their ordinary and customary meaning as would be understood by one of skill in the art in view of this disclosure, and include ones which are nontoxic to the cell or mammal being exposed thereto at the dosages and concentrations employed. Example “Pharmaceutically acceptable” carriers in accordance with methods and uses and compositions and product combinations herein can comprise, but not limited to, organic or inorganic, solid or liquid excipients which is suitable for the selected mode of application such as oral application or injection, and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as and nonionic surfactants such as TWEEN™ surfactant, polyethylene glycol (PEG), and PLURONICS™ surfactant. Auxiliary, stabilizer, emulsifier, lubricant, binder, pH adjustor controller, isotonic agent and other conventional additives may also be added to the carriers. In some embodiments, the composition is formulated for oral administration, rectal administration, or oral and rectal administration. In some embodiments, the composition and/or product combination comprises, consists essentially of, or consists of a probiotic.

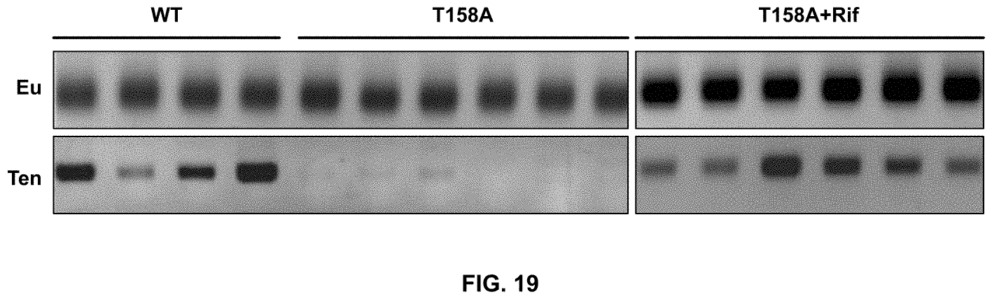

In some embodiments, any of the compositions and/or product combinations described herein comprises an antibiotic, for example a rifamycin (e.g., rifaximin), penicillin, or streptomycin (additional example antibiotics are described below). In some embodiments, the antibiotic of the product combination is in a separate composition that is separate from the bacteria. In some embodiments, the bacteria and antibiotic are together in a single composition. In some embodiments, the bacteria are together in a single composition, and the antibiotic is in a separate composition or set of compositions. In some embodiments, the bacteria are in two or more different compositions, and the antibiotic is in a separate composition or set of compositions. In some embodiments, the composition and/or product combination comprises an antibiotic that comprises, consists essentially of, or consists of rifaximin. Without being limited by theory, it is noted that rifaximin has been. shown to have gut-specific antibiotic effects. Moreover, it has been reported that treatment of human microbiota with rifaximin promotes the proliferation of probiotic species including Bifidobacteria (Maccafferii et al., 2010), which, as shown herein are reduced in Rett mice (See Example 8, FIG. 11.). Without being limited by theory, it is contemplated that rifaximin may exert its protective effect on Bifidobacteria by way of changes in the composition of other bacteria (See Example 18). Additionally it is shown herein that rifaximin increased abundance of the phylum Tenericutes, which is also reduced in Rett mice (Example 18 and FIGS. 11 and 19). Accordingly, in some embodiments, the compositions and/or product combination comprises an amount of rifaximin that is sufficient to increase an amount of Actinobacteria (e.g., Bifidobacteria) and/or Tenericutes bacteria upon administrate to a host, while having anti-bacterial effects on other bacteria. In some embodiments, the compositions and/or product combination comprises an amount of antibiotic that is sufficient to increase an amount of Actinobacteria (e.g., Bifodobacteria) and/or Tenericutes bacteria upon administrate to a host, while having anti-bacterial effects on other bacteria. In some embodiments, the composition and/or product combination comprises an amount of rifaximin that is sufficient to enhance the growth of Tenericutes bacteria in the subject. The composition or product combination can be provided in a unit dose comprising this amount. In some embodiments, the composition and/or product combination comprises an amount of rifaximin that is sufficient to enhance the growth of Bifodobacteria bacteria in the subject. The composition or product combination can be provided in a unit dose comprising this amount. In some embodiments, the composition and/or product combination comprises an amount of antibiotic that is sufficient to eliminate or substantially eliminate the gut microbiota of a subject. In some embodiments, the composition and/or product combination comprises an amount of antibiotic that is sufficient to reduce the overall quantity of gut microbes in the subject by at least 80%, for example at least 80%, 81%, 82%, 83%, 84%, 85%, 85%, 86%, 88%, 89%, 90%, 91%, 92%, 93%, 94%, 95%,96%, 97%, 98%, 99%, 99.5%, or 99.9%.

### Antibiotics

In some embodiments, any composition and/or product combination as described herein (including compositions and/or product combinations for uses and methods of reducing the likelihood of, delaying the onset of, or ameliorating one or more symptoms associated with Rett syndrome or Huntington's disease) comprises an antibiotic that is selected from the group consisting of: Amoxicillin, Amoxicillin/clavulanic acid (amoxicillin+clavulanic acid), Ampicillin, Benzathine benzylpenicillin, Benzylpenicillin, Cefalexin, Cefazolin, Cefixime, Cefotaxime, Ceftriaxone, Cloxacillin, Penicillin, Phenoxymethylpenicillin (penicillin V), Piperacillin/tazobactam, Procaine benzylpenicillin, Ceftazidimea, Meropenema, Aztreonama, Imipenem/cilastatin, Amikacin, Azithromycin[, Chloramphenicol, Ciprofloxacin, Clarithromycin, Clindamycin, Doxycycline, Erythromycin, Gentamicin, Metronidazole, Nitrofurantoin, Spectinomycin, Trimethoprim/sulfamethoxazole, Trimethoprim, Vancomycin, Clofazimine, Dapsone, Rifampicin, Ethambutol/isoniazid, Ethambutol/isoniazid/pyrazinamide/rifampicin, Ethambutol/isoniazid/rifampicin Isoniazid, Isoniazid/pyrazinamide/rifampicin, Isoniazid/rifampicin, Pyrazinamide, Rifabutin, Rifampicin, Rifa.pentine, Amikacin, Bedaquiline, Capreomycin, Clofazimine, Cycloserine, Delamanid, Ethionamide, Kanamycin, Levofloxacin, Linezolid, Moxifloxacin, p-aminosalicylic acid, rifabutin, rifapentine, rifalazil, rifaximin. Streptomycin, or a combination of two or more of these antibiotics. In some embodiments, any composition and/or product combination as described herein (including compositions and/or product combinations for uses and methods of reducing the likelihood of, delaying the onset of, or ameliorating one or more symptoms associated with Rett syndrome or Huntington's disease) comprises an antibiotic that is of the rifamycin class. Example antibiotics of the rifamycin class suitable for composition and/or product combination as described herein (including compositions and/or product combinations for uses and methods of reducing the likelihood of, delaying the onset of, or ameliorating one or more symptoms associated with Rett syndrome or Huntington's disease) include, but are not limited to, rifampicin (or rifampin), rifabutin, rifapentine, rifalazil and rifaxirnin.

Methods of Reducing the Likelihood of, Delaying the Onset of, or Ameliorating One or More Symptoms Associated with Rett Syndrome or Huntington's Disease Comprising Administering Bacteria

Some embodiments include methods of reducing the likelihood of, delaying the onset of, or ameliorating one or more symptoms associated with Rett syndrome or Huntington's disease in a subject in need thereof. The method can comprise administering a composition or product combination comprising bacteria as described herein to the subject. In some embodiments, the method comprises administering to the subject a composition or product combination comprising, consisting essentially of, or consisting of one or more bacteria selected from the group consisting of: Actinobacteria bacteria (e.g., Bifidobacteria such as Bifidobacterium choerinum), Tenericutes bacteria (e.g., Mesoplasma bacteria such as Mesoplasma entomophilum), and Bacteroides bacteria (e.g., B. fragilis, B. ovatus, and/or B. thetaiotaomicron), or a combination of two or more of these. In some embodiments, Actinobacteria bacteria (e.g., Bifidobacteria such as Bifidobacterium choerinum) and Tenericutes bacteria (e.g., Mesoplasma bacteria such as Mesoplasma entomophilum) are administered to the subject. In some embodiments Actinobacteria bacteria (e.g., Bifidobacteria such as Bifidobacterium choerinum) and Bacteroides bacteria (e.g., B. fragilis, B. ovatus, and/or B. thetaiotaomicron, or a combination of two or more of these, for example, B. fragilis and B. ovatus; B. fragilis and B. thetaiotaomicron; B. ovatus, and B. thetaiotaornicron; or B. fragilis, B. ovatus, and B. thetaiotaomicron) are administered to the subject. In some embodiments Bifidobacteria and Bacteroides bacteria (e.g., B. fragilis, B. ovatus, and/or B. thetaiotaomicron, or a combination of two or more of these, for example, B. fragilis and B. ovatus; B. fragilis and B. thetaiotaomicron; B. ovatus, and B. thetaiotaomicron; or B. fragilis, B. ovatus, and B. thetaiotaomicron) are administered to the subject. In some embodiments, the method comprises administering a composition or product combination comprising bacteria and an antibiotic (e.g., any antibiotic as described herein, for example, a rifamycin such as rifampicin (or rifampin), rifabutin, rifapentine, rifalazil or rifaximin) as described herein to the subject. In some embodiments, the bacteria are administered in a single composition. In some embodiments, the bacteria are administered in a product combination, and the components of the product combination can be administered at the same time or at different times. In some embodiments, the bacteria are administered in a product combination, and the components of the product combination can be administered together (e.g., in a mixture) or separately (the separate administrations can be at the same times or at different times). In sonic embodiments, the method reduces the likelihood, delays the onset of, or ameliorates one or more symptoms associated with Rett syndrome, for example, impaired motor function, gastrointestinal complications, excess abdominal fat, elevated levels of lipopolysaccharide (LPS) in the circulation, small brain, and/or reduced levels of neurotrophies. In some embodiments, the method is for reducing the likelihood of, delaying the onset of, or ameliorating one or more symptoms associated with Rett syndrome, and the method enhances neurogenesis and dendritic arborization in the hippocampus and/or in the subventricular zone, and/or reduces astrocyte and microglial activation. In some embodiments, the method reduces the likelihood, delays the onset of, or ameliorates one or more symptoms associated with Huntington's disease. Example symptoms associated with Huntington's disease that can be reduced in likelihood, delayed in onset, or ameliorated include impaired motor function, and/or aggregates of mutant Huntingtin protein in the CNS. In some embodiments, the method enhances neurogenesis and dendritic arborization in the central nervous system (e.g., hippocampus and/or in the subventricular zone), and/or reduces astrocyte and microglial activation in a Huntington's disease patient. In some embodiments, the subject is a human.

In some embodiments, the method further comprises selecting the subject as being within a class of subjects that should receive the composition (or product combination) for reducing the likelihood of, delaying the onset of, or ameliorating one or more symptoms associated with Rett syndrome or Huntington's disease. For example, a profile of a sample of a subject as described herein can indicate a presence or elevated risk of Rett syndrome or Huntington's disease. Accordingly, in sonic embodiments, the method further comprises determining a profile of a sample of a subject (as described herein) in order to select the subject as being within a class of subjects that should receive the composition (or product combination). In some embodiments, the subject is identified as a member of a subset of individuals with or at risk of Rett syndrome that should receive the composition (or product combination). In some embodiments, the subject is a member of a subpopulation of subjects with Rett syndrome that are amenable to treatment with a composition and/or product composition comprising bacteria and/or an antibiotic as described herein. The identification can be based on the gut microbiota composition of the subject (which can be measured, for example, by determining a profile of a sample of a subject as described herein). In some embodiments, the subject is identified as a member of a subset of individuals with or at risk of Huntington's disease that should receive the composition (or product combination). In some embodiments, the subject is a member of a subpopulation of subjects with Huntington's disease that are amenable to treatment with a composition and/or product composition comprising bacteria and/or an antibiotic as described herein. The identification can be based on the gut microbiota composition of the subject (which can be measured, for example, by determining a profile of a sample of a subject as described herein).

In some embodiments, the method reduces the likelihood of, delays the onset of, or ameliorates the one or more symptoms associated with Huntington's disease, and a composition or product combination comprising, consisting essentially of, or consisting of Bacteroides bacteria is administered to the subject. The Bacteroides bacteria can comprise, consist essentially of, or consist of B. fragilis, B. ovatus, and/or B. thetaiotaomicron, or a combination of two or more of these as described herein. In some embodiments, the composition or product combination further comprises Actinobacteria bacteria (e.g., Bifidobacteria, such as Bifidobacterium choerinum) as described herein.

In some embodiments, the method reduces the likelihood of, delays the onset of, or ameliorates the one or more symptoms associated with Huntington's disease, and a composition or product combination comprising, consisting essentially of, or consisting of Actinobacteria bacteria is administered to the subject. In some embodiments, the Actinobacteria bacteria comprise, consist essentially of, or consist of Bifidobacteria, for example, Bifidobacterium choerinum. In some embodiments, the composition or product combination further comprises Bacteroides bacteria (e.g., B. fragilis, B. ovatus, and/or B. thetaiotaomicron, or a combination of two or more of these, such as B. fragilis and B. ovatus; B. fragilis and B. thetaiotaomicron; B. ovatus, and B. thetaiotaomicron; or B. fragilis, B. ovatus, and B. thetaiotaomicron).

In some embodiments, the method reduces the likelihood of, delays the onset of, or ameliorates the one or more symptoms associated with Rett syndrome, and a composition or product combination comprising, consisting essentially of, or consisting of Actinobacteria bacteria (e.g., Bifidobacteria such as Bifidobacterium choerinum) and Bacteroides bacteria (e.g., B. fragilis, B. ovatus, and/or B. thetaiotaomicron, or a combination of two or more of these, such as B. fragilis and B. ovatus; B. fragilis and B. thetaiotaomicron; B. ovatus, and B. thetaiotaomicron; or B. fragilis, B. ovatus, and B. thetaiotaomicron) is administered to the subject. In some embodiments, the composition or product combination comprises, consists essentially of, or consists of Bifidobacteria and Bacteroides bacteria.

In some embodiments of the method, the composition or product combination comprising, consisting essentially of, or consisting of the Actinobacteria bacteria (e.g., Bifidobacteria) and the Bacteroides bacteria as described herein is administered to the subject. In some embodiments, the Actinobacteria bacteria and the Bacteroides bacteria are administered to the subject simultaneously. In some embodiments, the Actinobacteria bacteria and the Bacteroides bacteria are administered to the subject separately. In some embodiments, the Actinobacteria bacteria and the Bacteroides bacteria can be administered to the subject simultaneously or separately. In some embodiments, the Actinobacteria bacteria comprise, consist essentially or, or consist of Bifidobacteria. In some embodiments, the Bacteroides bacteria are selected from the group consisting of: B. fragills, B. ovatus, and B. thetaiotaomicron, or a combination of two or more of these (for example, B. fragilis and B. ovatus; B. fragilis and B. thetaiotaomicron; B. ovatus, and B. thetaiotaomicron; or B. fragilis, B. ovatus, and B. thetaiotaomicron) as described herein.

In some embodiments of the method, the composition or product combination comprising, consisting essentially of, or consisting of the Actinobacteria bacteria (e.g., Bifidobacteria) and the Tenericutes bacteria as described herein is administered to the subject. In some embodiments, the Actinobacteria bacteria and the Tenericutes bacteria are administered to the subject simultaneously. In some embodiments, the Actinobacteria bacteria and the Tenericutes bacteria are administered to the subject at different times. In some embodiments, the Actinobacteria bacteria and the Tenericutes bacteria are administered to the subject simultaneously or at different times. In some embodiments, the Actinobacteria bacteria comprise, consist essentially or, or consist of Bifidobactiera.

In some embodiments of the method, the composition or product combination administered to the subject comprises, consists essentially of, or consists of bacteria that map to an OTU that maps to a bacterium selected from the group consisting of Mesoplasma entomophilum, Lactobacillus taiwanensis, Pediococcus argentinicus, Bifidobacterium choerinum. As noted in Table 1, each of these listed bacteria is underexpressed in Rett syndrome mice compared to wild-type (control) mice. In some embodiments, a bacteria maps to an OTU when the bacteria comprise a 16S rRNA sequence of at least 100 nucleotides that is least 97% identical to a reference 16S rRNA sequence of the OTU, for example at least 97%, 98%, or 99% identical.

In some embodiments of the method, no more than 10⁶ du of Firmicutes bacteria is administered to the subject. In some embodiments, no more than10⁵ cfu, 10⁵ cfu, 10³ cfu, 10² cfu, or 10 cfu of Firmicutes bacteria is administered to the subject. In some embodiments, the composition or product combination administered to the subject is substantially free, or is free of Firmicutes bacteria. As noted in Example 8, Firmicutes is significantly increased in the gut of Rett syndrome mice, and as such, without being limited by theory, is contemplated that compositions that are free or substantially free of Firmicutes may help to direct the gut microbiota away from a Rett profile.

A number of routes of administration are contemplated for the composition and/or product combination in accordance with methods described herein. In some embodiments of the method, the composition and/or product combination is administered to the subject via oral administration, rectum administration, transdermal administration, intranasal administration, intravenous administration, subcutaneous administration, and/or inhalation.

Methods of Reducing the Likelihood of, Delaying the Onset of, or Ameliorating One or More Symptoms Associated with Rett Syndrome or Huntington's Disease Comprising Administering Antibiotics

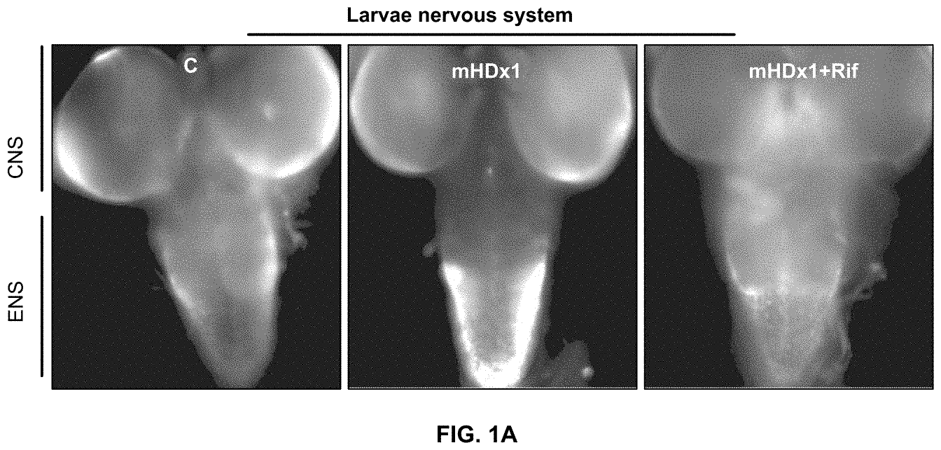

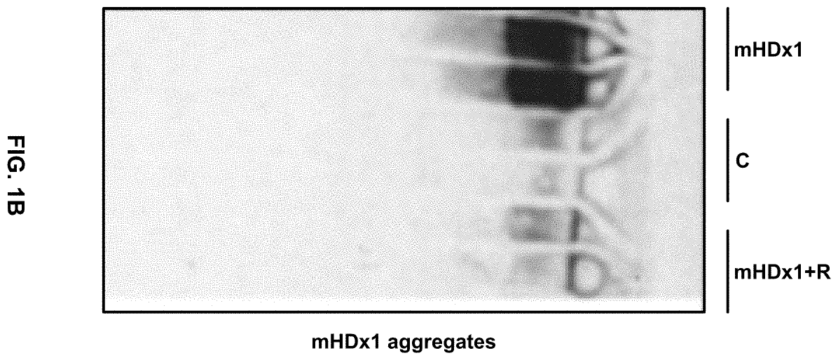

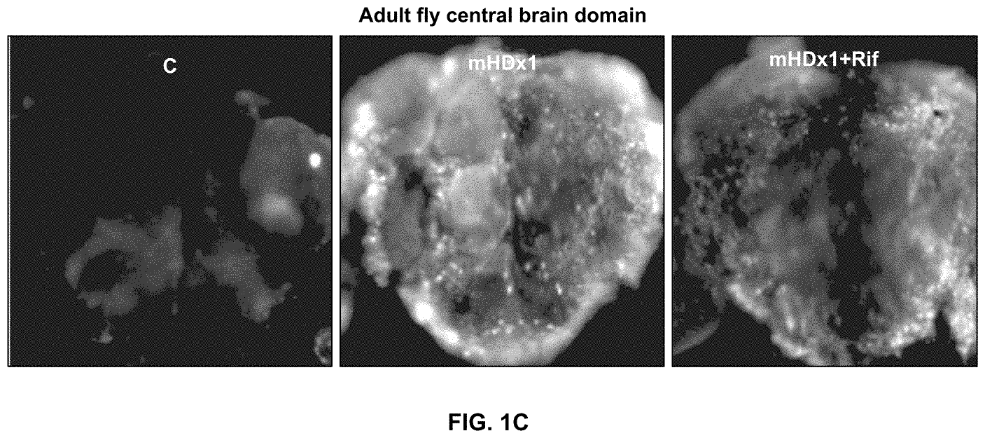

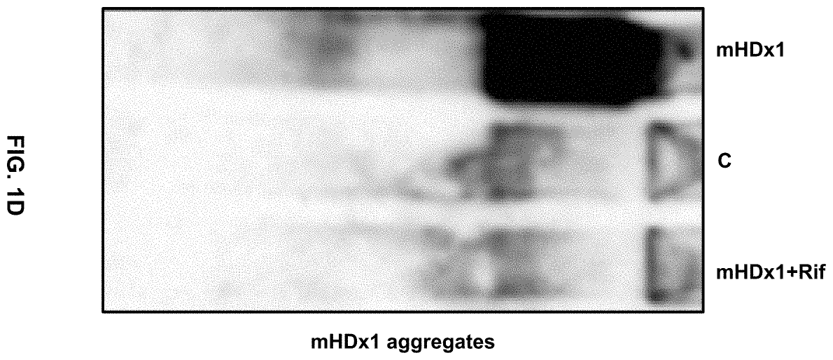

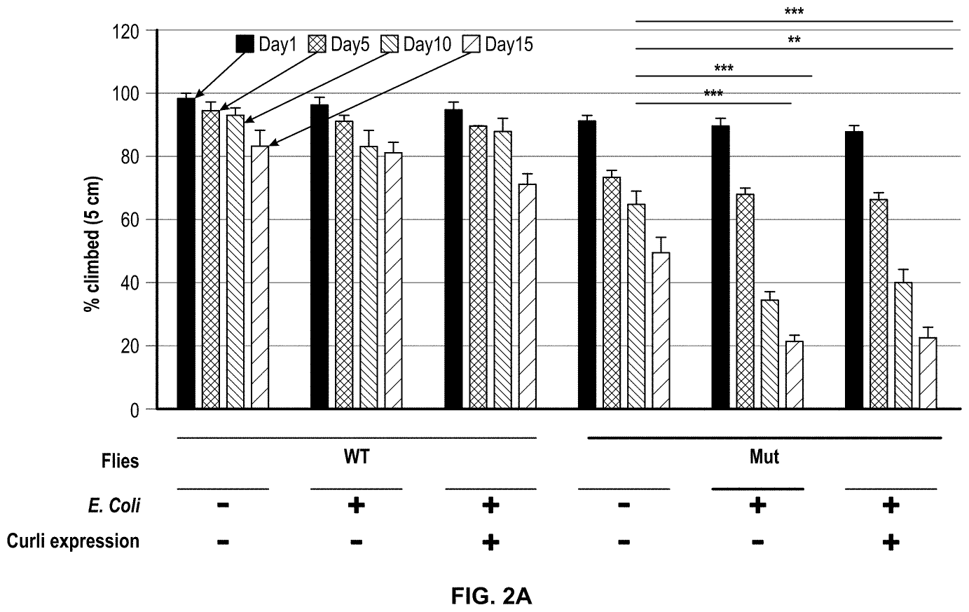

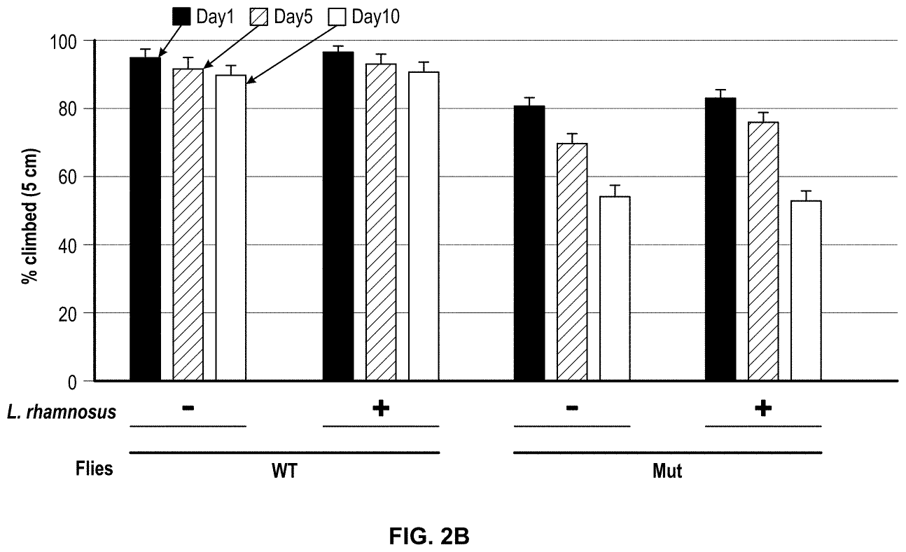

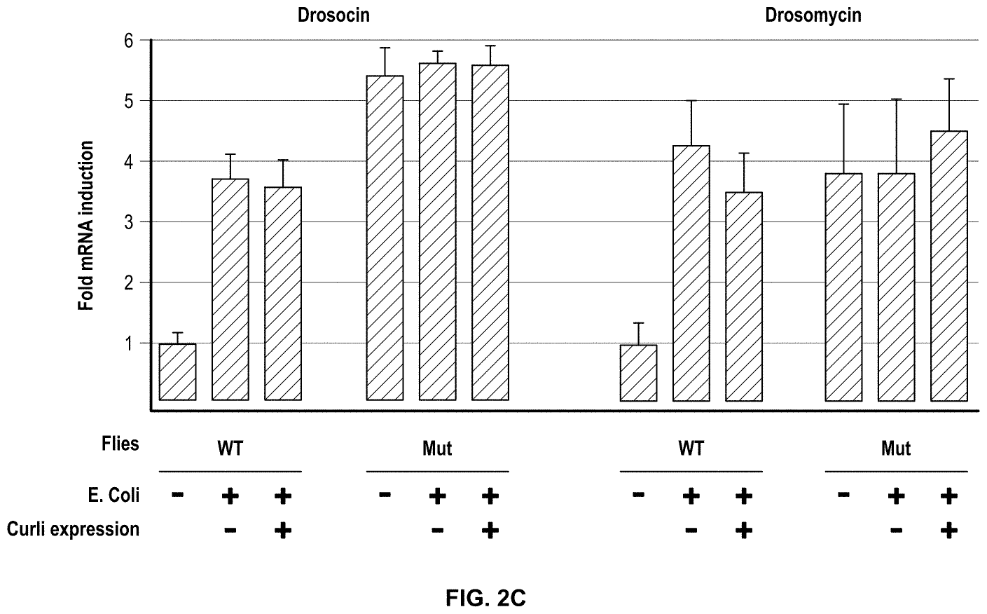

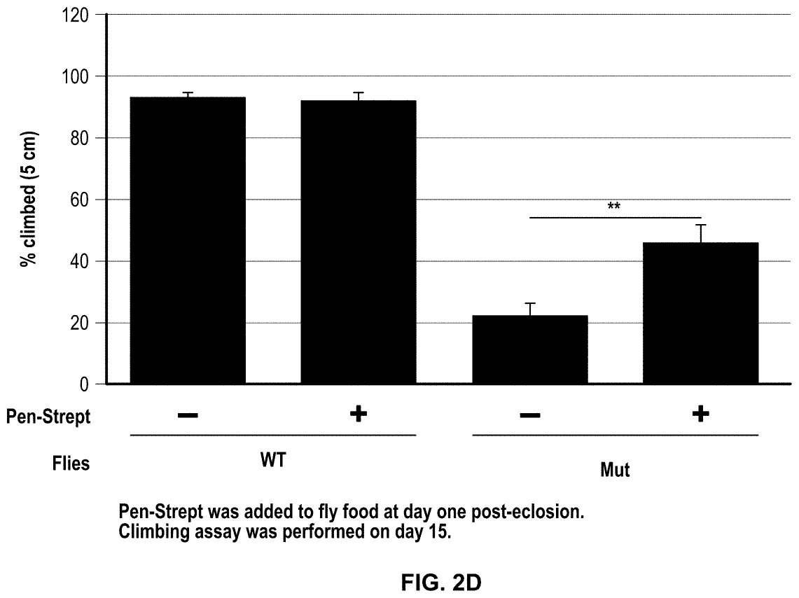

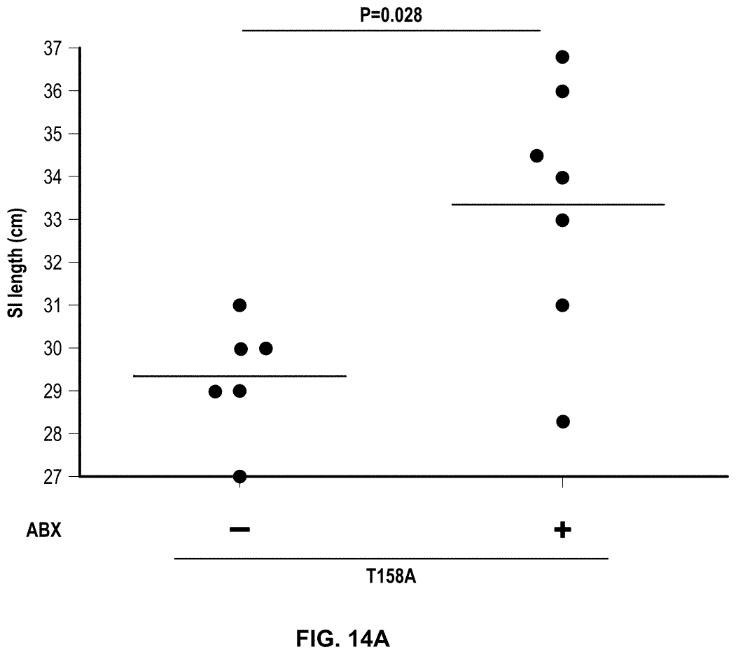

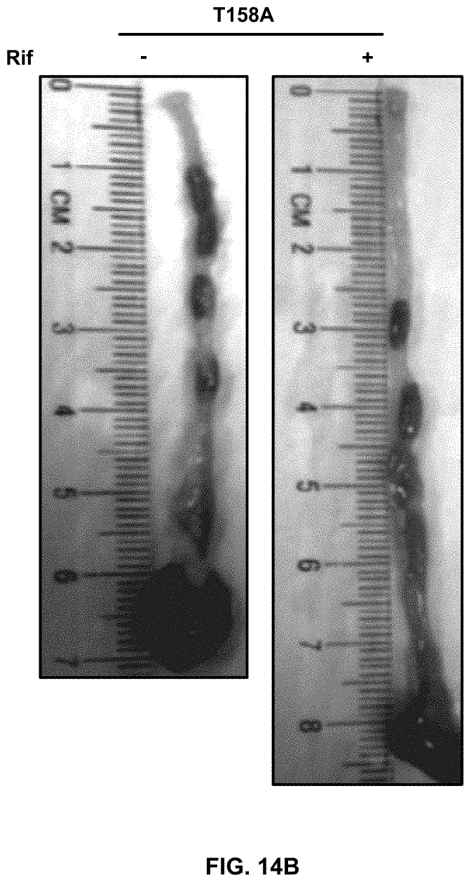

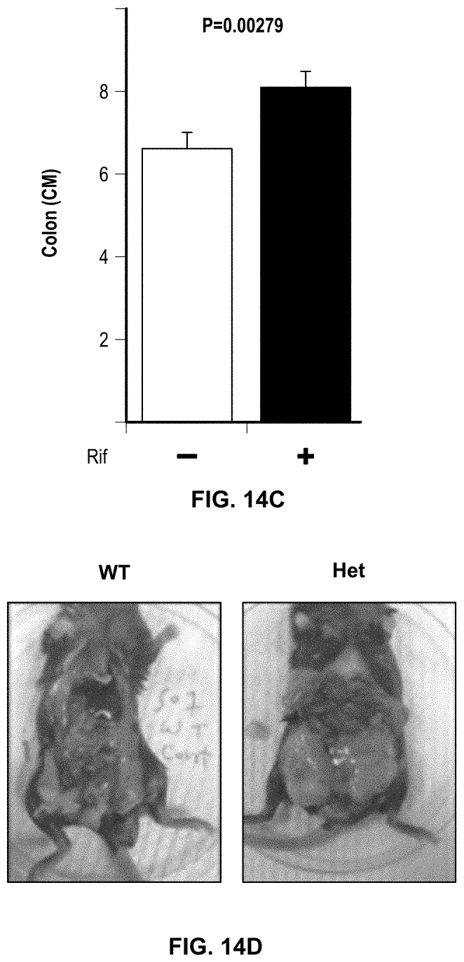

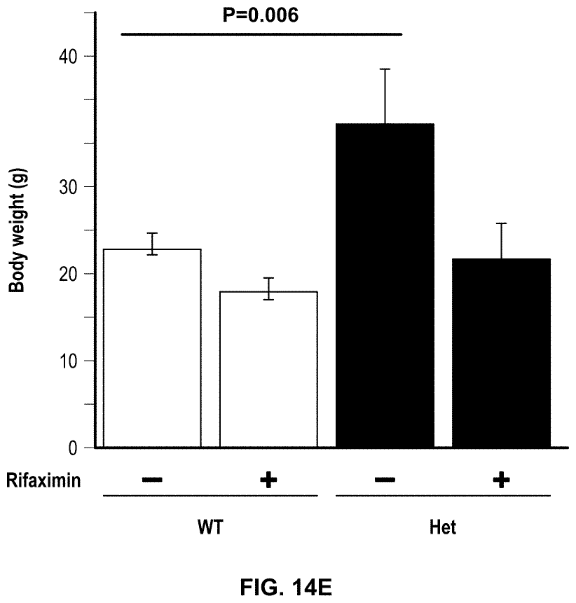

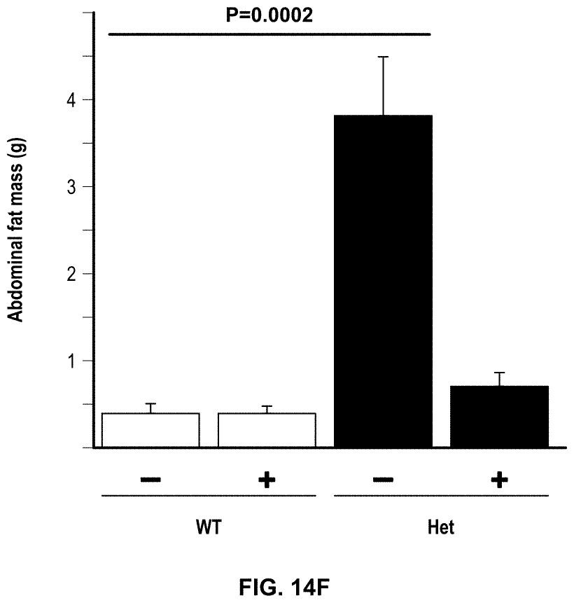

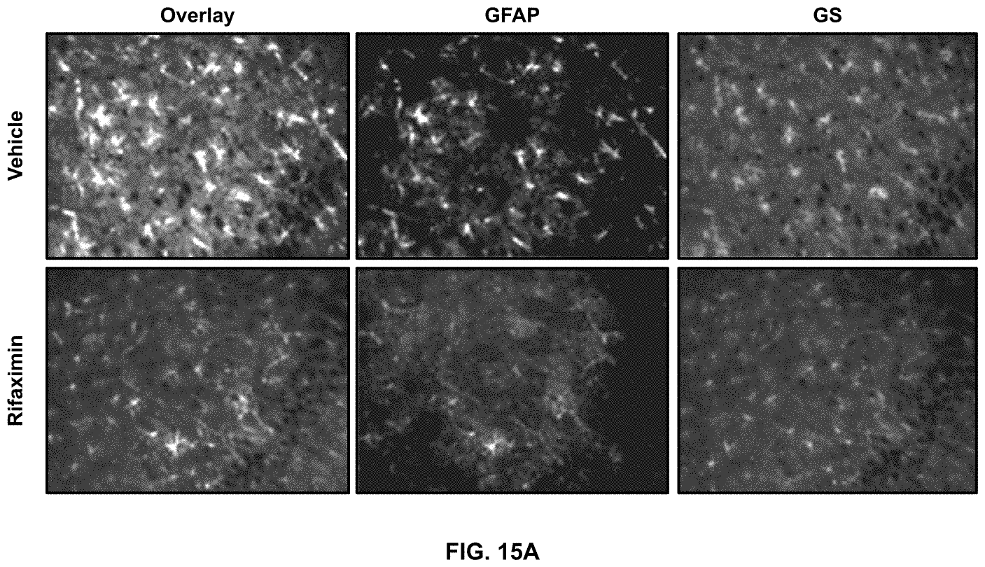

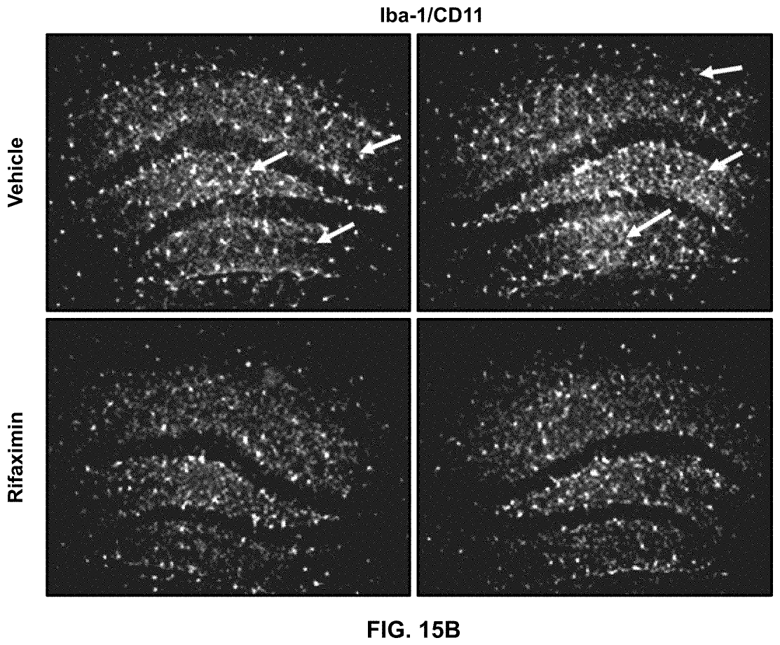

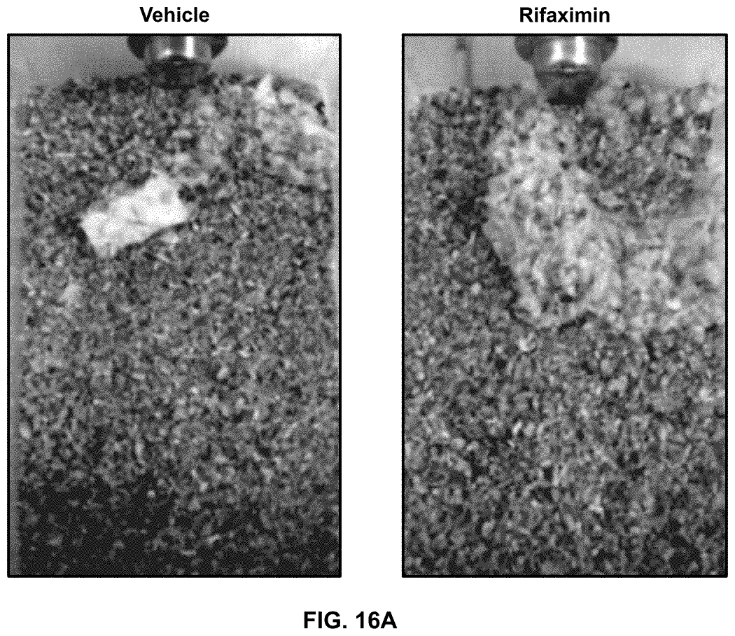

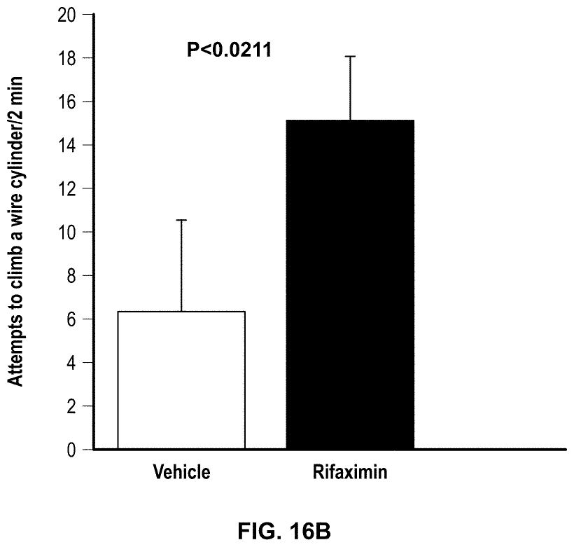

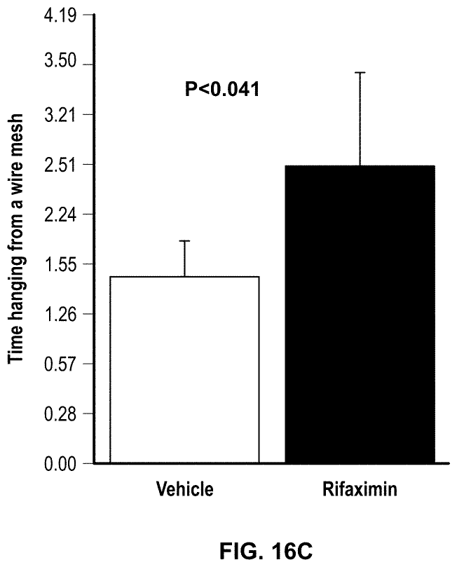

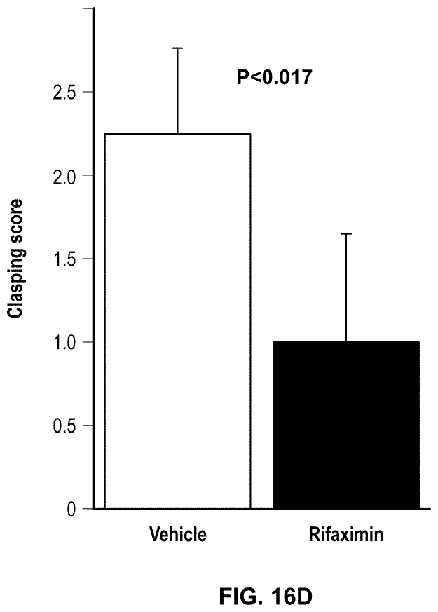

It is reported herein that administering an antibiotic can ameliorate symptoms of genetic models of Rett syndrome (See Example 1445) and Huntington's disease (See Examples 1-2). In particular, reducing or eliminating gut microbial species by treating Rett mice with rifaximin for 6 weeks after weaning has been shown to improve motor symptoms in a Rett model, as treated animals make more attempts to climb a wire-meshed cylinder, display enhanced muscle strength, and have reduced clasping phenotype (Example 16, FIGS. 16B-D), Furthermore, treating Rett mice with rifaximin reduced staining for biomarkers such as glial fibrillary acidic protein (GFAP) and glutamine synthetase (GS) when compared to vehicle-treated cohorts (Example 15 and FIG. 15A). Furthermore, treatment of Rett mice post-weaning with rifaximin for 6 weeks selectively elongates the colon and prevents intestinal obesity (Example 14, and FIGS. 14 B-C). Treating a Drosophila genetic model of Huntington's disease with antibiotics (including rifaximin) also lowered aggregation of mutant Huntingtin (mHDx1) protein in the CNS of larval and adult flies and improved motor symptoms of Huntington's disease (Example 3, FIG. 1). Similar improvement of motor symptoms was observed in Huntington's disease Drosophila that received the antibiotics penicillin-streptomycin (Example 3, FIG. 2D). On the other hand, administering E. coli to the Drosophila model accelerated the motor symptoms of HD (Example 3, FIG. 2A), In some embodiments, the subject is a human.

Accordingly, some embodiments include a method of reducing the likelihood of, delaying the onset of, or ameliorating one or more symptoms associated with Rett syndrome or Huntington's disease in a subject in need thereof, the method comprising administering an antibiotic (e.g., any antibiotic as described herein, for example, a rifamycin such as rifampicin (or rifampin), rifabutin, rifapentine, rifalazil or rifaximin) as described herein to the subject. In some embodiments, the antibiotic comprises, consists of, or consists essentially of rifaximin. In some embodiments, the antibiotic comprises, consists of, or consists essentially of an antibiotic as described herein, or a combination of two or more antibiotics as described herein, for example penicillin and streptomycin. In some embodiments, the method reduces the likelihood, delays the onset of, or ameliorates one or more symptoms associated with Rett syndrome, for example, impaired motor function, gastrointestinal complications, excess abdominal fat, elevated levels of lipopolysaccharide (LPS) in the circulation, small brain, and/or reduced levels of neurotrophins. In some embodiments, the method is for reducing the likelihood of, delaying the onset of, or ameliorating one or more symptoms associated with Rett syndrome, and the method enhances neurogenesis and dendritic arborization in the hippocampus and/or in the subventricular zone, and/or reduces astrocyte and microglial activation. In some embodiments, the method reduces the likelihood, delays the onset of, or ameliorates one or more symptoms associated with Huntington's disease. Example symptoms associated with Huntington's disease that can be reduced include impaired motor function and/or aggregates of mutant Huntingtin protein in the CNS.

In some embodiments, the method further comprises selecting the subject as being within a class of subjects that should receive a composition or product combination for reducing the likelihood of, delaying the onset of, or ameliorating one or more symptoms associated with Rett syndrome or Huntington's disease. For example, the method can comprise determining a profile of a sample of the subject as described herein. The profile can indicate whether the subject has or is at risk of Rett syndrome and or Huntington Disease, and whether the subject would benefit from the composition or product combination comprising, consisting of, or consisting essentially of antibiotic. In sonic embodiments, the subject is a post-weaning child.

In some embodiments, administering the antibiotic reduces the quantity of gut bacteria in a subject by at least 80%, for example at least 80%, 81%, 82%, 83%, 84%, 85%, 85%, 86%, 88%, 89%, 90%, 91%, 92%, 93%, 94%, 95%,96%, 97%, 98%, 99%, 99.5%, or 99.9%. In some embodiments, administering the antibiotic renders the gut substantially free of bacteria. In some embodiments, administering the antibiotic protects or increases Bifidobacteria and/or Tenericutes in the gut of the subject, but reduces overall gut microbes in the subject by at least 80%, for example at least 80%, 81%, 82%, 83%, 84%, 85%, 85%, 86%, 88%, 89%, 90%, 91%, 92%, 93%, 94%, 95%,96%, 97%, 98%, 99%, 99.5%, or 99.9%.

In some embodiments, the method further comprises administering a composition or product combination comprising, consisting of, or consisting essentially of bacteria selected from the group consisting of: Actinobacteria bacteria, Tenericutes bacteria, and Bacteroides bacteria, or a combination of two or more of the listed bacteria to the subject. The composition or product combination can be as described herein. In some embodiments, the composition or product combination comprises one or more bacteria that map to an OTU that maps to any of Mesoplasma entomophilum, Lactobacillus taiwanensis, Pediococcus argentinicus, Bifidobacterium choerinum. As described herein, in some embodiments, bacteria map to an OTU when the bacteria comprise a 16S rRNA sequence of at least 100 nucleotides that is least 97% identical to a reference 16S rRNA sequence of the OTU. In some embodiments, Actinobacteria bacteria and Bacteroides bacteria are administered to the subject. In some embodiments, the Actinobacteria bacteria are administered to the subject, and wherein the Actinobacteria bacteria comprises Bifidobacteria. In some embodiments, the Bacteroides bacteria are administered to the subject, wherein the Bacteroides bacteria are selected from the group consisting of: B. fragilis, B. ovatus, and B. thetaiotaomicron, or a combination of two or more of the listed bacteria. In some embodiments, the bacteria and antibiotic are administered together. In some embodiments, the bacteria and antibiotic are administered separately. In some embodiments, the bacteria and antibiotic are administered at the same time. In some embodiments, the bacteria and antibiotic are administered at the same time, and in the same compositions. In some embodiments, the isolated bacteria and antibiotic are administered at the same time, but in separate compositions. In some embodiments, the bacteria and antibiotic are administered at different times. In some embodiments, the antibiotic is administered prior to the bacteria. In some embodiments, the bacteria is administered prior to the antibiotic. In some embodiments, the composition or product combination is substantially free of Firmicutes bacteria. In some embodiments, no more than 10⁶, 10⁵, 10⁴, 10³, 10², or 10 cfu of Firmicutes bacteria is administered to the subject.

### Methods of Determining a Profile of a Sample of a Subject

In some embodiments, a method of determining a profile of a sample of a subject is provided. The method can comprise detecting at least one of:
(a) a presence and/or level of a gut bacterium selected from the group consisting of: Tenericutes, Actinobacteria, and Firmicutes, or a combination of two or more of the listed bacteria;
(b) a serum level of a neurotransmitter selected from the group consisting of Choline, 5-HT, Tyrosine, Dopamine, and Epinepherine, or two or more of the listed neurotransmitters, or
(c) an expression level of a cholinergic gene selected from the group consisting of: Chrna2, Chma7, Chrb4, Chrm1, Slc5a7, Chat. Ache, and Slc18a3, or two or more of the listed genes.

The profile can comprise the detected presence and/or levels of (a); (b); (c); (a) and (b); (a) and (c); (b) and (c); or (a) and (b) and (c). In some embodiments, the subject is a human.

Regarding (a), it is observed that in the colon contents of subjects with Rett syndrome, levels of Tenericutes, and Actinobacteria are decreased and Firmicutes are increased relative to levels in a control (non-Rett) subject (See Example 8). Furthermore, Bifidobacteria (a genus of Actinobacteria) were absent from the GI tract of Rett mice starting at 6 weeks of age (See FIG. 11C), an age prior to the typical appearance of Rett symptoms. Accordingly, in some embodiments, the method of determining the profile comprises determining (a), and the sample comprises gut and/or feces material of the subject, and a profile comprising higher levels of Firmicutes or lower levels of Tenericutes and/or Actinobacteria relative to a non-Rett (control) sample indicates an elevated risk of Rett syndrome. It can further indicate that the subject is a member of a subpopulation of subjects with Rett syndrome that are amenable to treatment with a composition and/or product composition comprising bacteria and/or an antibiotic as described herein. The non-Rett control sample can comprise, consists essentially of, or consist of gut and/or feces material of a non-Rett individual. In sonic embodiments, the levels of bacterial phyla, genera, and/or species of the non-Rett control sample are provided as stored values, for example electronically stored values.

In some embodiments of the method, the Actinobacteria comprise Bifidobacteria. In some embodiments, the method of determining the profile comprises determining (a), and the subject's sample comprises gut and/or feces material of the subject. An absence or substantial absence of Bifidobacteria from the subject's sample (e.g., less than 5%, of the amount of Bifidobacteria in a non-Rett control sample) indicates a presence or an elevated risk of Rett syndrome. It can further indicate that the subject is a member of a subpopulation of subjects with Rett syndrome that are amenable to treatment with a composition and/or product composition comprising bacteria and/or an antibiotic as described herein.

In some embodiments, the levels of Tenericutes, Actinobacteria and/or Firmicutes, and/or the presence or absence of Bifidobacteria is determined by nucleic acid testing, for example qualitative PCR, semi-quantitative or quantitative PCR, nucleic acid sequencing, microarray analysis, or the like. For example, an absence or substantial absence of Bifidobacteria in the gut and/or feces sample of the subject can be determined by detecting a Bifidobacteria in a of a non-Rett control sample. In some embodiments, the method comprises detecting levels of bacterial phyla, genera, and/or species in the non-Rett control sample.

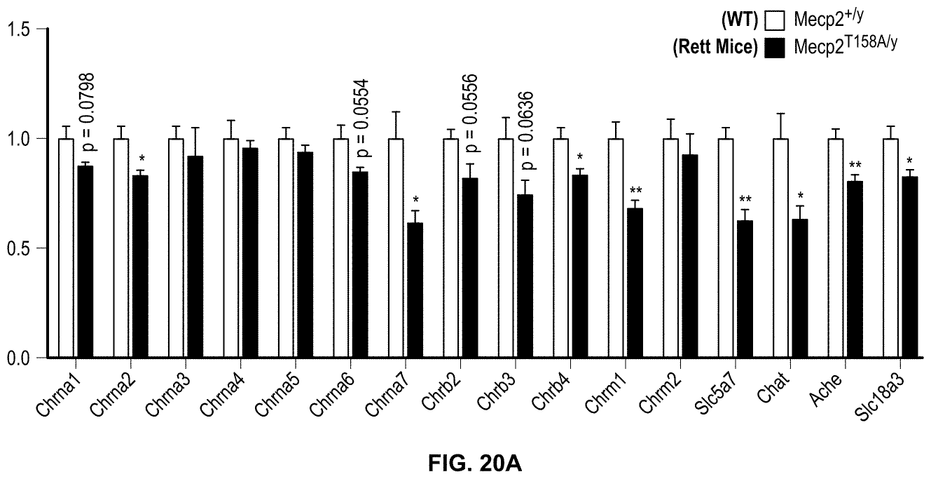

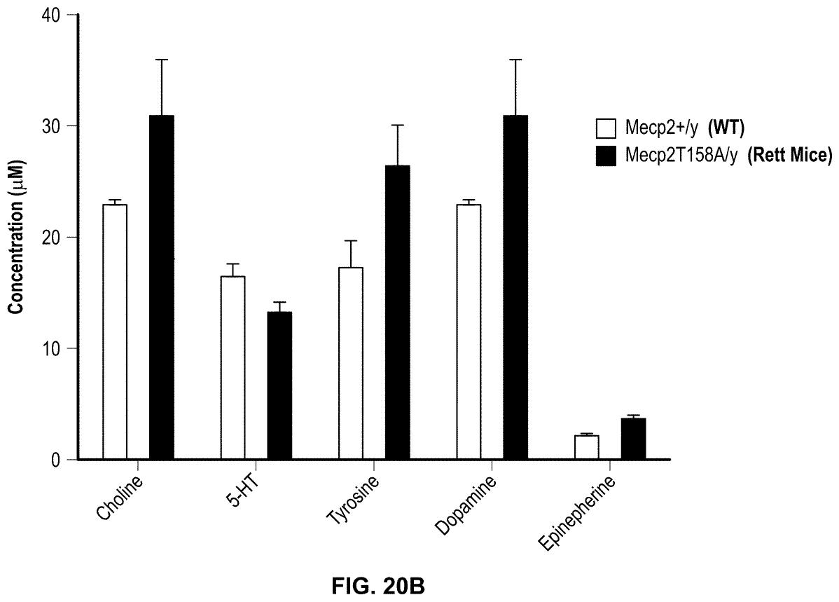

Regarding (b), it is observed that serum levels of certain neurotransmitters are altered in Rett mice compared to wild-type (non-Rett) controls (See Example 20 and FIG. 20A). In particular, the serum levels of choline, tyrosine, dopamine, and epinephrine were observed to be decreased in the serum of Rett mice, and the serum levels of serotonin were observed to be increased in the serum of Rett mice (FIG. 20A). In some embodiments, the method of determining the profile comprises determining (h), and the subject's sample comprises serum of the subject. A higher serum level of choline, tyrosine, dopamine, and/or epinephrine, and/or a lower serum level or serotonin in the subject sample, compared to a non-Rett (control) sample can indicate a presence or an elevated risk of Rett syndrome. It can further indicate that the subject is a member of a subpopulation of subjects with Rett syndrome that are amenable to treatment with a composition and/or product composition comprising bacteria and/or an antibiotic as described herein. The non-Rett control sample can comprise, consists essentially of, or consist of serum of a non-Rett individual. In methods and uses of some embodiments herein, the serum levels of choline, tyrosine, dopamine, and/or epinephrine can be measured using a number of suitable techniques, for example immunoassays such as ELBA, lateral flow, and/or no-wash assays; mass spectrometry such as gas chromatography mass spectrometry (GC-MS), mass spectrometry-mass spectrometry (MS/MS), or MALDI (Matrix Assisted Laser Desorption/Ionization); or nuclear magnetic resonance (NMR). In some embodiments, the serum levels of choline, tyrosine, dopamine, and/or epinephrine are measured in a non-Rett control sample. In some embodiments, the serum levels of choline, tyrosine, dopamine, and/or epinephrine of the non-Rett control sample are provided as stored values, for example electronically stored values.

Regarding (c), it is observed that expression levels of certain cholinergic genes are altered in Rett mice compared to wild-type (non-Rett) controls) (See Example 20 and FIG. 20B). In particular, expression levels of Chrna2, Chrna7, Chrb4, Chrm1, Slc5a7, Chat, Ache, and Slc18a3 are reduced in Rett mice compared to wild-type controls (Example 20 and FIG, 20B). In some embodiments, the method of determining the profile comprises determining (c), and the subject's sample comprises gut tissue such as a biopsy or swab and levels of cholinergic gene expression are measured. Lower levels of Chrna2, Chrna7, Chrb4, Chrm1, Slc5a7, Chat, Ache, Slc18a3, or a combination of two or more of any of the listed genes in the subject sample, as compared to a non-Rett control can indicate an increased risk of Rett syndrome. It can further indicate that the subject is a member of a subpopulation of subjects with Rett syndrome that are amenable to treatment with a composition and/or product composition comprising bacteria and/or an antibiotic as described herein. The non-Rett control sample can comprise gut tissue of a non-Rett individual. A number of techniques can be used to measure expression levels of cholinergic genes, for example nucleic acid assays such as quantitative reverse-transcriptase PCR, microarray analysis, and the like. In some embodiments, the expression levels of cholinergic genes are measured in a non-Rett control sample. In some embodiments, the expression levels of cholinergic genes of the non-Rett control sample are provided as stored values, for example electronically stored values.

In some embodiments, the profile is determined, and if the profile indicates an increased risk or Rett syndrome, the subject is recommended for a treatment. The treatment can comprise administering to the subject a composition and/or product combination comprising bacteria, antibiotic, or bacteria and antibiotic as described herein.

### Additional Embodiments

The human gut microbiota contains approximately 100 trillion bacterial cells, which matures during the first 2-3 years of life and is critical for health and immune development (Round et al., 2009, Goyal et al., 2015). Changes in the homeostasis of intestinal rnicrobiota (dysbiosis) are implicated in cancer, obesity, malnutrition, abnormal gut and immune system development, inflammatory bowel disease (IBD), diabetes, neurodevelopmental and neuropsychiatric disorders, multiple sclerosis (MS), Parkinson's disease (PD), and Alzheimer's disease (AD) (Scheperjans, et al., 2015, Blanton et al., 2016, Marchesi et al., 2016, Minter et al., 2016, Sherwin et al., 2016). The signaling pathways connecting the gut microbiota to CNS functions are not well defined. Molecules produced by intestinal bacteria may directly influence the biology of the enteric nervous system (ENS), which communicates with the CNS via the vagus nerve. Microbial metabolites may also reach the brain via the circulation or affect the CNS through communication with the immune system (Goyal et al., 2015, Sherwin et al., 2016), Germ-free (GF) mice have been instrumental in studying the impact of gut microbes on brain development. Behavioral studies demonstrate that GF animals are hyperactive and display reduced anxiety. GF mice have altered brain chemistry with changes in the expression of neurotransmitter and proteins essential for synaptogenesis such as BDNF, and have enhanced hippocampal neurogenesis (Ogbonnaya et al. 2015, Luczynski et al. 2016). Recent findings suggest that GF PD mice have reduced brain pathology and motor symptoms compared to conventionally housed littermates. GE mice accumulate less of insoluble α-synuclein, fewer activated microglia, and lower levels of inflammatory biomarkers. An observation was made that microbiota of PD patients accelerate the progression of motor symptoms in PD mice suggesting the presence of potentially pathogenic bacteria (Sampson and Mazmanian et al., 2016). Treatment of AD mice with a cocktail of antibiotics reduces beta-amyloid formation and neuroinflammation (Minter et al., 2016). Without being limited by theory, it is contemplated that gut microbiota may influence neurodegenerative disorders.

Normalizing intestinal dysbiosis offers therapeutic strategies Normalizing intestinal dysbiosis offers therapeutic strategies to positively impact brain development and ameliorate aberrant behaviors. Colonization with Bacteroides fragilis, a commensal bacterium of the human gut, improves CNS-related symptoms in an environmental mouse model of autism (Hsiao et al., 2013). Similarly, oral administration of distinct Bifidobacteria is protective in animal models of neuropsychiatric disorders (Savignac et al., 2015). PD mice treated with antibiotics have reduced microglial activation and CNS pathology (Sampson et al., 2016). Antibiotics also enhance the therapeutic effects of levodopa and improve the clinical symptoms of HD patients (Hashim et al., 2014). Changing the intestinal microbiota of Hepatic Encephalopathy (HE) patients with a non-absorbable antibiotic ameliorates aberrant behaviors including cognitive defects, mood disorders, and motor symptoms (Bajaj et al., 2013). HE is a model of gut-liver-brain axis disease where intestinal dysbiosis intoxicates the liver, leading to brain pathology. Without being limited by theory, these studies underscore the significance of a healthy intestinal microbiota for brain function and support the notion that altering the intestinal microbiota may be a safe and noninvasive strategy to treat brain disorders. In addition to CNS pathology, HD patients suffer from weight loss/muscle atrophy, metabolic and immune abnormalities, and GI complications (Carrol et al., 2015). The links between microbiota and the emergence of similar symptoms have been established in other brain disorders models including PD (Scheperjans, et al., 2015, Blanton et al., 2016, Dinan et al., 2016). Without being limited by theory, it is contemplated that gut microbiota may regulate HD pathology.

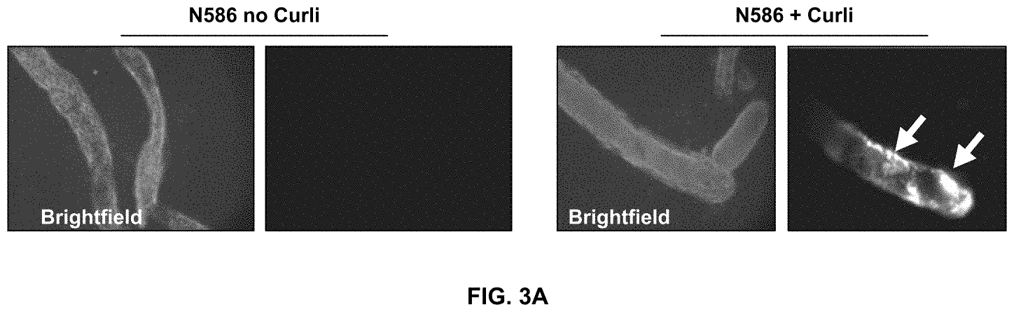

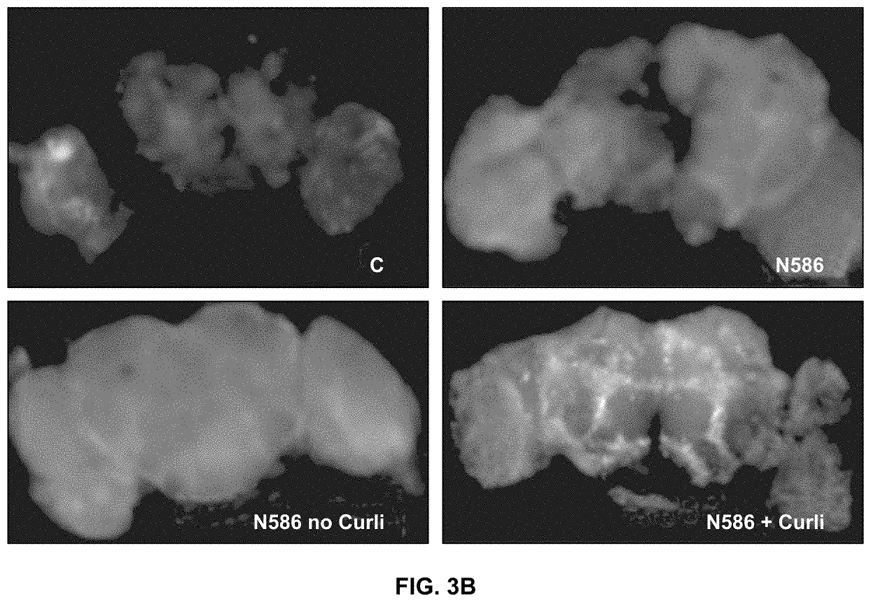

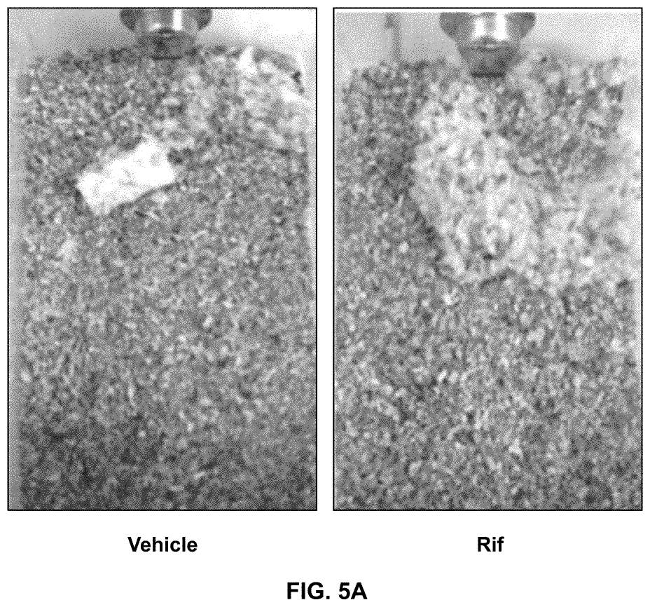

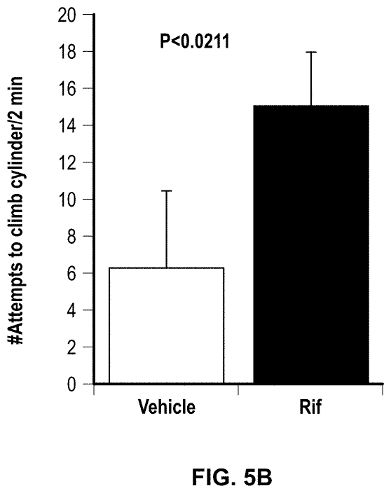

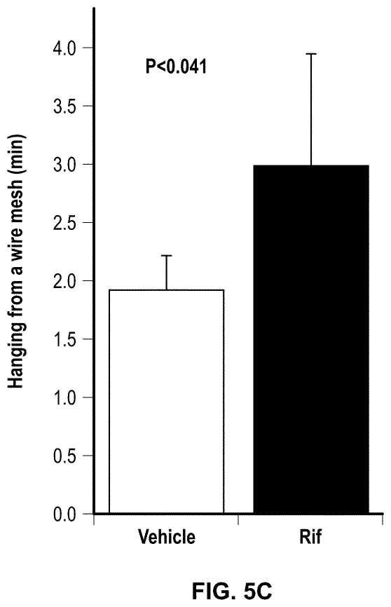

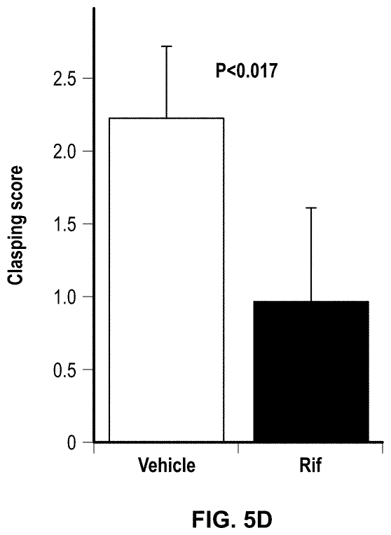

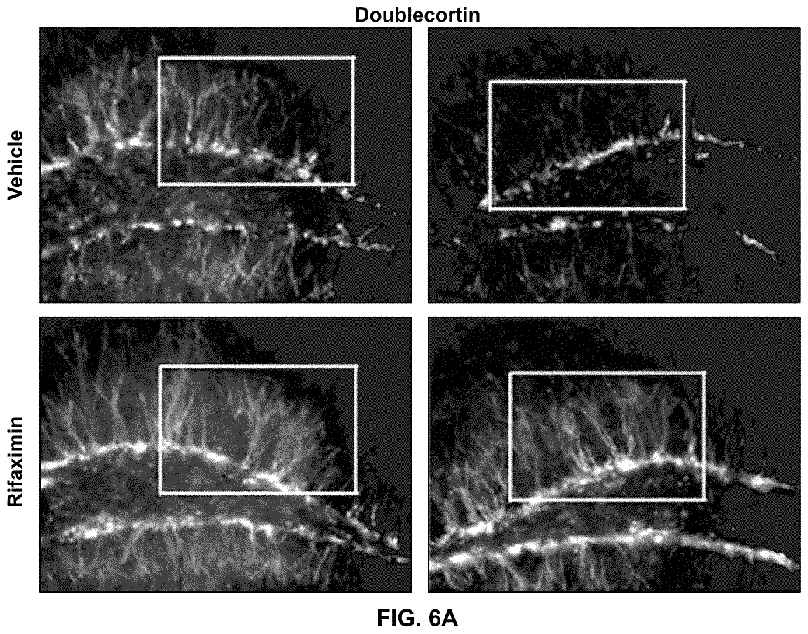

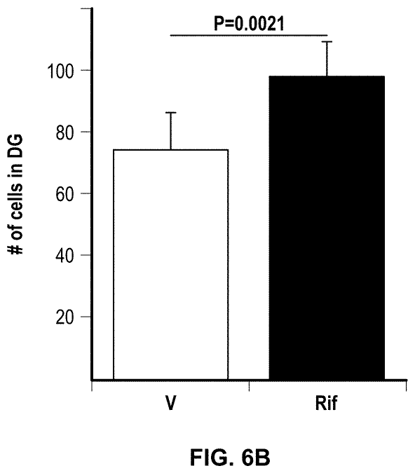

The HD research field is frequently approached by a “CNScentric” approach for finding therapeutic targets. However, most neurodegenerative disorders including HD are syndromic, which may involve defects in various organs and pathways (Carrol et al., 2015, Titova et al 2016). The discovery of microbiota-brain interactions has raised the intriguing question of whether the gut environment influences neurodegeneration. HD with a known genetic cause is an excellent model to dissect the impact of intestinal flora on protein aggregation and the progression of CNS-related symptoms. Results from a Drosophila HD model reported herein (see Example 3) demonstrate that the mutant HTT aggregates appear first in neurons in the vicinity of the intestinal tract. Moreover, elimination of intestinal microbiota with the clinically useful antibiotic rifaximin lowers the oligomerization of mutant HTT exon-1 (mHDx1). Conversely, colonization of HD flies with a strain of E. coli, which produce functional bacterial amyloids, accelerates the oligomerization of an N-terminal 586 fragment and exacerbates the motor symptoms. The impact of microbiota on the mutant HTT aggregation and HD pathogenesis in a mouse model is also contemplated. One approach is developing a germ-free colony to examine whether intestinal microbiota influence the oligomerization of mutant HTT and the progression of HD symptoms. In a Rett syndrome mouse model, which displays motor and cognitive impairments, it is observed herein that rifaximin promotes hippocampal neurogenesis and dendritic arborization, and reduces the severity of motor symptoms (see Example 6, FIGS. 3 and 5).

Additionally, changes in the BDNF levels, synaptogenesis, and neurogenesis contribute to the progression of Rett symptoms (Rarnocki et al., 2008, Lombardi et al., 2015).

Normalizing intestinal dysbiosis offers therapeutic strategies to positively impact brain development and ameliorate abnormal behaviors in accordance with some embodiments herein. Colonization with Bacteroides fragilis, a commensal bacterium of the human gut, improves CNS-related symptoms in an environmental mouse model of autism (Hsiao et al., 2013). Similarly, oral administration of distinct Bifidobacteria is protective in animal models of neuropsychiatric disorders (Savignac et al., 2015). Changing the intestinal microbiota of Hepatic Encephalopathy (HE) patients with a non-absorbable antibiotic ameliorates aberrant behaviors including cognitive defects, mood disorders, and motor symptoms(Bajaj et al., 2013). HE is a model of gut-liver-brain axis disease where intestinal dysbiosis intoxicates the liver, leading to brain pathology. These studies underscore the significance of a healthy intestinal microbiota for optimal brain function and support the notion that correcting dysbiosis maybe a safe and noninvasive strategy to treat various brain disorders.

Rett syndrome offers genetic model to study the influence of microbiota on brain development since pathologies such as small brain, impaired neurogenesis and synapse formation, reduced levels of neurotrophins, and motor symptoms are well defined parameters. The Rett mouse models develop most of the symptoms displayed by patients thus facilitating translation of any therapeutic strategies to humans. Finally, Rett syndrome can be described as an autism spectrum disorder with Parkinsonian symptoms, anxiety disorder, seizure, and intellectual disability. Accordingly, the knowledge and therapeutic strategies of this disclosure are further consistent with additional data on neurological diseases, for example data described herein.

MeCP2 is a multifunctional protein expressed in several organs. As a DNA binding transcription factor, MeCP2 affects the expression of hundreds of genes implicated in neurodevelopment and immune cell functions (Crony et al., 2015, Lombardi et al., 2015). However, MeCP2 is best known as a global epigenetic regulator recognizing methylated and hydroxy-methylated DNA in the regulatory domains in the genome (Mellen et al., 2012). MeCP2 activity and DNA binding is regulated by posttranslational modifications such as phosphorylation (Ebert et al., 2013). These properties of MeCP2 are critical for the proper functioning of numerous neuronal circuits, growth factor production, and signal-induced synaptogenesis (Lombardi et al., 2015). MeCP2 has been extensively studied in the CNS. MeCP2 maintains the homeostasis of lipids and cholesterol in the liver. Consistent with this observation, cholesterol lowering compounds such as statins have been reported to reduce the severity of symptoms in Rett mice (Buchovecky et al., 2013, Kyle et al., 2016). Some Rett patients display abnormal lipid and cholesterol profiles. Accordingly, it is contemplated herein that manipulating metabolic pathways (for example by altering the gut microbiota in accordance with some embodiments herein) may benefit some of the Rett patients. There are also reports that MeCP2 can regulate immune development and the production of inflammatory cytokines, as other potential comorbidity factors in Rett (Li et al., 2014, Jiang et al., 2014, Crook et al., 2015, O'Driscoll et al., 2015). Inflammation contributes to the development of autistic symptoms in mouse models and treatment with a commensal probiotic Bacteroides fragilis reduces the production of inflammatory cytokines and ameliorates aberrant behaviors (Hsiao et al, 2013, Mayer et al., 214).

It has been reported that MeCP2 activity is regulated by a chromatin modifying, serine/threonine kinase IKKα (IκB kinase α), which is also a component of the multi-subunit IKK complex modulating inflammatory pathways and immune development (Chariot. 2009). IKKα phosphorylates MeCP2 and promotes its interaction with CREB (cAMP response element-binding protein) transcription factor and subsequently influences the expression of ˜300 neuronal genes such as BDNF and the synapse-associated protein PSD-95 (Khoshnan et al., 2012). In order to study the neuroimmune pathways, a Rett mouse model with a WT immune system was engineered using Cre-lox technology. However, it is reported herein that expression of MeCP2 in the immune system of Rett mice did not affect the progression of symptoms (unpublished data). Several other laboratories have arrived at similar conclusions demonstrating that bone marrow transplants or reactivating MeCP2 selectively in the innate immune cells does not ameliorate the Rett symptoms (Wang et al., 2015). Also engineered was a Rett mouse model with selective knockdown (KD) of IKKβ, which is a prominent proinflammatory kinase inducing NF-κB-dependent production of the majority of inflammatory cytokines in the immune system and other organs (Chariot, 2009). However, it is reported herein that KD of IKKβ exacerbated the Rett symptoms and promoted muscle wasting (Khoshnan et al, unpublished data). Recent studies however indicate that NF-κB activity, which is downstream of IKKβ, is elevated in the CNS of Rett mice and genetic reduction of NF-κB ameliorates brain pathology, aberrant behaviors, and extends the life span (Kishi et al., 2016).

The majority of Rett patients suffer from GI complications such as constipation, abdominal pain, bloating, and difficulty with defecation (Motil et al., 2012). Moreover, a recent study suggests that Rett patients have altered intestinal microbiota (Strati et al., 2016). It is reported herein that MeCP2 is expressed in the intestinal epithelium including the progenitor cells involved in gut development. Without being limited by theory, it is contemplated that MeCP2 deficiency in the intestinal epithelium of Rett patients may alter the gut physiology and microbial homeostasis. In support of this hypothesis, it is observed that Rett mice display GI pathology, barrier permeability known as “leaky gut”, and reduced number of intestinal cells critical for the production of antimicrobial peptides. Rett mice also exhibit dysbiosis exemplified by the lack of specific health-promoting bacterial species. GI inflammation and metabolic changes such as excess abdominal fat and elevated levels of bacterial lipopolysaccharide (LPS) in the circulation are other notable pathologies in Rett mice. Notably, GI and metabolic defects are absent in the germ-free Rett mice or are reduced by changing the intestinal microbiota of conventionally raised cohorts. Treatment with a non-absorbable antibiotic rifaximin, which reduces the overgrowth of harmful bacteria in the small intestine, induces beneficial biochemical and behavioral changes in Rett mice. Of interest, rifaximin enhances neurogenesis and dendritic arborization in the hippocampus and in the subventricular zone, reduces astrocyte and microglial activation, and improves motor function (See Example 6. FIGs, 3 and 5).

The finding that MeCP2 is expressed in the intestinal epithelium suggests that it may be important for gut physiology. Dysbiosis, GI pathology, and metabolic changes in Rett mice are consistent with this notion. Engineering a gut-only MeCP2 mouse model will facilitate investigating the role of intestinal MeCP2 in the microbiota-gut-brain networks relevant to Rett syndrome. Without being limited by theory, it is contemplated that reactivation of MeCP2 in the intestinal epithelium of Rett mice could significantly restore the gut-microbiota communications thus reducing the propagation of dysbiotic species and enhancing colonization by health-promoting organisms. Possible candidates mediating gut homeostasis include the production of antimicrobial peptides and growth factors, which may be regulated by MeCP2. Alternatively, MeCP2 may dampen the inflammatory response of the immune and epithelial cells and thus reduce pathology. A healthy gut environment could also ameliorate the metabolic and systemic changes including those caused by the leakage of microbial products and metabolites such as LPS in the circulation, which can accumulate in the CNS and cause neuroinflammation. Correcting the gut physiology may also enhance the overall wellbeing of animals and potentially reduce some of the CNS pathology. A positive outcome of these studies will be the knowledge essential to correct the gut environment of Rett patients and lower the severity of symptoms. A negative result will support CNS-mediated GI pathology, metabolic changes, and dysbiosis in Rett.

Without being limited by theory, it is contemplated that the regulation of MeCP2 expression and activity in the intestinal epithelium is critical for maintaining the gut-microbiota homeostasis, MeCP2 deficiency in the gut leads to dysbiosis and GI pathology, which could disrupt gut-immune and gut-brain communications, alter brain chemistry, and exacerbate the progression of Rett-related symptoms.

Without being limited by theory, it is contemplated that intestinal microbiota contributes to GI pathology, metabolic and immune abnormalities, and aberrant behaviors in Rett mice. The established. GF Rett mouse colony described herein can be useful in elucidating the role of microbiota in these phenotypes. GE mice display altered expression of CNS genes such as BDNF, which is a target of MeCP2 (Bercik et al., 2011, Chahrour et al., 2008). Moreover, components of microbiota can influence MeCP2 activity (Bie et al., 2013). Thus, without being limited by theory, it is contemplated that the experiments described herein can further identify MeCP2-dependent genes and pathways that are different between GE and conventionally raised animals. Based on the existing knowledge of GF mice, but without being limited by theory, it is contemplated that motor and affective behaviors of Rett mice will be different between the conventional and GF models (Luczynski et al. 2016). The GF Rett mice can be used for testing relevant probiotic bacteria to correct distinct behaviors. It is further contemplated that the neuroprotective and gut-immune modulating properties of B. fragilis may correct some of the pathology in Rett mice. On the long term however, we may be able to test other commensal bacteria once we get a better picture of the intestinal microbiota in Rett mice. The impacts of rifaximin on correcting some of the GI pathology, metabolic changes and potentially CNS symptoms are encouraging. Testing existing therapeutics has an advantage since they could be tested in patients with less stringent regulations. Rifaximin has a proven record of safety and efficacy for treating microbiota-mediated GI and CNS pathology in humans. Experiments described herein testing its properties in mouse models strongly support its applicability to Rett patients.

Some experiments described herein may provide significant knowledge on the gut-hippocampus interactions and could identify the associated pathways and neuronal genes influenced by the microbiota-MeCP2 interactions. Recent studies suggest that eliminating the intestinal microbiota with a cocktail of antibiotics impairs hippocampal neurogenesis and cognitive tasks in WT mice further supporting a gut-hippocampus network Möhl et al., 2016). However, some of the antibiotics in those experiments are toxic and enter the circulation. Rifaximin is different since it is gut-specific and behaves as a modifier of microbiota, changing the ratios of colonized organisms (Maccafferii et al., 2010, Ponziani et al., 2016). Data in FIG. 19 are also consistent with this notion. The proven beneficial effects of rifaximin on cognition and working memory in HE patients (Ahluwalia et al., 2014) add credence and optimism for future evaluation of this compound in Rett. Considering the presence of intellectual disability in Rett syndrome, a positive outcome of these experiments could yield a therapy to tackle this debilitating symptom in the near future. Probing the microbiota of rifaximin-treated Rett mice may also identify commensal bacteria, which could regulate hippocampal neurogenesis and influence cognition. Without being limited by theory, if successful, such strategies may have therapeutic implications beyond Rett syndrome since impaired neurogenesis is present in several brain disorders including autism, fragile-X syndrome, Alzheimer's disease, and traumatic brain injury.

### EXAMPLES

### Example 1

### Effects of Rifaximin on a Drosophila Model of HD

Without being limited by theory, it is contemplated that intestinal microbiota contributes to the aggregation of mutant HTT in the enteric nervous stem (ENS). In a Drosophila model of HD (Barbaro et al., 2015), inducing the expression of a mutant HTT exon-1 fragment (mHDx1) in the nervous system promoted aggregation in the ENS of larvae in the vicinity of the gut (FIGS. 1A and 1B). The initial appearance of aggregates in the larval ENS is thus observed.

The bacterial content of adult HD flies is dramatically increased compared to controls. Without being limited by theory, this suggests that neuronal mutant HTT may impact the composition of intestinal rnicrobiota. Moreover, it is observed that larvae generated in the presence of rifaximin, a gut-specific antibiotic, lowers the aggregation of mHDx1 in (FIGS. 1A and 1B). Rifaximin also reduces the aggregation of mHDx1 in the CNS of adult flies (FIGS. 1C and 1D). Accordingly, it is observed herein that treatment with an antibiotic in accordance with some embodiments herein decreases aggregates of mutant Huntingtin protein.

### Example 2

### Effects of Bacterial Components on a Drosophila Model of HD

It was studied whether bacterial components affect the oligomerization of mutant HTT. HD flies (expressing mutant human huntingtin protein), which express the first 586 N-terminal amino acids of mutant HTT (586 HD (120Q), do not display any motor symptoms (Barbaro et al., 2015). Colonization of the 586 HD gut with an E. coli strain producing functional bacterial amyloids Curli (FIG. 3A right panels), accelerates the aggregation of the N-586 mutant HIT fragments (FIGS. 3B-C), Moreover, Curli+ bacteria exacerbate the motor symptoms of the 586 flies since they lose the ability to climb a cylindrical vial (FIG. 3D). Thus, bacterially-expressed Curli in the gut can accelerate the development of motor symptoms in HD.

On the other hand, feeding E. coli (both wild-type and Curil-expressing MC4100) to HD flies accelerates the development of motor systems. Notably, E. coli (5×10⁷ CFU/ml) was added to fly food on Day 1 for wild-type (“WI”) flies and mutant (“Mut”) flies expressing mutated human huntingtin. Climbing behavior was assessed on Days 1, 5, 10, and 15. The E. coli exacerbated the aberrant motor behavior of the HD flies, so that they exhibited more severe motor symptoms at Days 10 and 15 than HD flies that did not receive E. coli (See FIG. 2A). The Curli-expressing E. coli and E. coli mutant lacking Curli had similar effects. Thus, E. coli in the gut (Curli-expressing, or non-Curli-expressing) can exacerbate motor symptoms in HD.

Feeding Lactobacillus rhamnosus to HD flies did not affect motor symptoms. L. rhamnosus at 5×10⁷ CFU/ml was added to fly food at day 1 post-eclosion for wild-type (“WT”) flies and mutant (“Mut”) flies expressing mutated human huntingtin. The climbing assay was performed on days 1, 5, and 10. The L. rhamnosus had no appreciable effect on motor symptoms as measured by the climbing assay (See FIG. 2B).

Overall, these findings demonstrate that modulation of the gut environment in accordance with some embodiments herein can modulate the motor symptoms of a HD model in vivo. Without being limited by theory, it is contemplated that the gut bacteria may influence the aggregation of mutant HTT in the nervous system and potentially influence the downstream pathology.

HD flies (expressing mutated human huntingtin) further exhibited elevated levels of the mRNAs encoding the antimicrobial peptides (MMPs) Drosocin and Drosomycin (See FIG. 2C). mRNA was quantified by TR-qPCR and adjusted to WT flies with no bacteria. Flies were fed no E. coli, MC4100 E. coli expressing Curli, or mutant isogenic E. coli with Curli deleted. Feeding E. coli to wild-type (“WT”) flies induced the expression of the antimicrobial peptides drosocin and droscomycin. However, drosocin and droscomnycin were elevated in HD expressing mutated human huntingtin (“Mut”) flies that did not receive E. coli, and the addition of E. coli had no appreciable effect on drosocin and drosomycin mRNA levels. Moreover, the E. coli had comparable effects, regardless of whether they expressed Curli (See FIG. 2C).

Elimination of gut microbes by feeding penicillin-streptomycin ameliorated motor symptom defects in HD flies. Penicillin-streptomycin was added to fly food at day 1, post-eclosion. The climbing assay was performed on day 15. The HD flies that received penicillin-streptomycin exhibited significantly superior climbing ability compared to HD flies that did riot receive the penicillin-streptomycin (See FIG. 2D). Accordingly, it is concluded that treatment with antibiotics (for example to reduce or eliminate gut microbes) accordance with sonic embodiments herein can ameliorate motor symptoms defects in HD.

### Example 3

### Effects of Microbes on a Mouse Model of HD

### HD Mouse Model.

The R6/2 HD mouse line, which expresses the mutant HTT exon-1 (mHDx1) fragment, is a popular model and displays robust symptoms of HD including motor and cognitive impairments. Symptoms appear 6-8 weeks after birth (Mangiarini et al., 1996). Moreover, similar to HD patients, R 6/2 mice lose weight, and show immune cell abnormalities, It is relevant that R6/2 mice also display GI defects such as impaired gut motility, diarrhea, and malabsorption of nutrients (van der Burg, et al., 2011). The majority of these phenotypes are linked to disruption in the homeostasis of intestinal microbiota in other disease models. Thus, R6/2 can be used as a model to explore the impact of intestinal microbiota in HD. RNA sequencing of gut microbiome (total bacterial genomes) is performed on R6/2mice, and effects on intestinal microbiota in HD mice are observed.

Germ-free (GF) mice are tools to dissect the gut-brain interactions in disease models. A GF colony of the R6/2 model is generated. Mice are routinely tested for the presence of microbes by culturing under aerobic and anaerobic conditions and PCR.

Once the colony is established, longitudinal immunohistochemical studies of the gastrointestinal (GI) tract sections starting at postnatal day 0 (P0) up to 6 weeks (P42) are performed, using various anti-aggregate/oligomer-specific antibodies similar to those used in FIGS. 1 and 2. Brain sections of same age animals are also stained and aggregation of mHDx1 in the ENS and/or CNS is detected, as is the timing of when aggregates appear. It is expected that effects of HD on the GI tract in this model are observed.

Studies further suggest that intestinal microbiota may affect motor behavior in a PD mouse model (Sampson & Ma.zmanian et al., 2016). Without being limited by theory, it is contemplated that microbiota may also impact the motor symptoms in HD flies (See FIG. 2D). Thus, the motor performance of conventional and GF HD mice is observed in relation to WT littermates. Rotarod, beam crossing, and clasping are performed as described for HD mice previously (Southwell et al., 2009). A description of these tests is provided in Example 19.

Hippocampal neurogenesis is implicated for learning and memory in mammals, and may be influenced by intestinal microbiota (Christian et al., 2014, Ogbonnaya, et al. 2015). It is observed herein that impaired hippocampal neurogenesis in the conventional Rett syndrome mice is ameliorated in the GF cohorts, suggesting a link between gut microbiota and cognition (See, e.g., Examples 6, 17). Hippocampal neurogenesis is also impaired in the R6/2 HD mice (Fedele et al., 2011). Using BrdU labeling, the newly generated neurons in the conventional and GF WT and HD mice are examined, and survival time and integration into brain circuits is observed. Brain sections are stained with antibodies to markers of proliferation (ki67) and doublecortin, which stains the newly generated neurons. Behaviors such as novel object recognition and Barnes Maze are tested, and effects on to learning and memory of GF HD are compared to the conventional cohorts. These studies elucidate whether intestinal microbiota in HD mice contributes to impaired hippocampal neurogenesis and cognitive tasks.

Rotarod, beam crossing, clasping, open field, novel object and Barnes maze are performed by standard procedures as described (Dawood et al., 2004, Southwell et al., 2009, Hsiao et al., 2013).

### Example 4

### Effects of Microbiota on Myelination

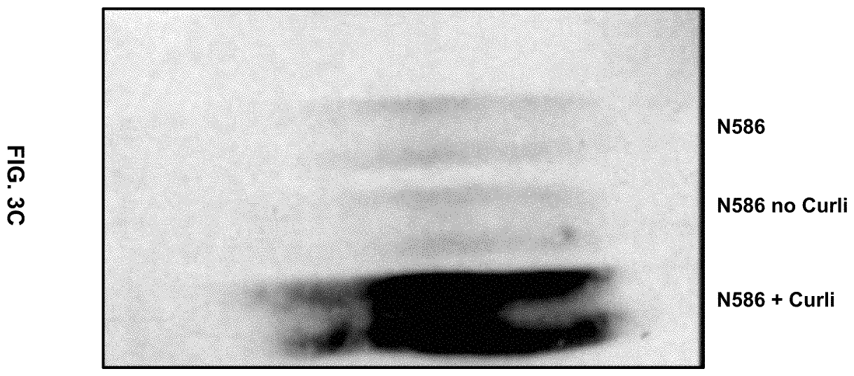

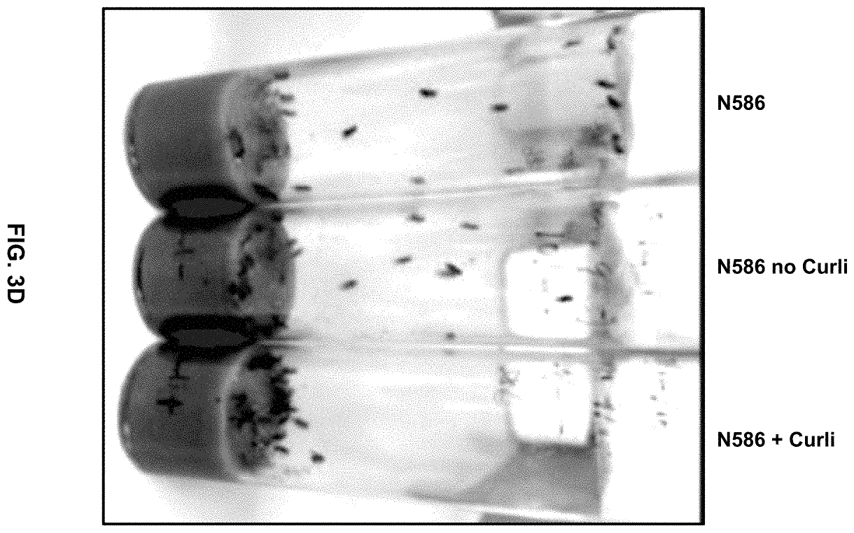

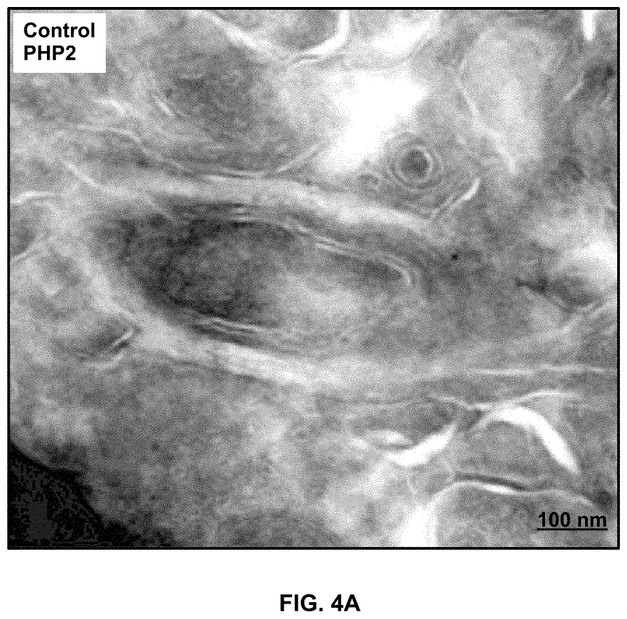

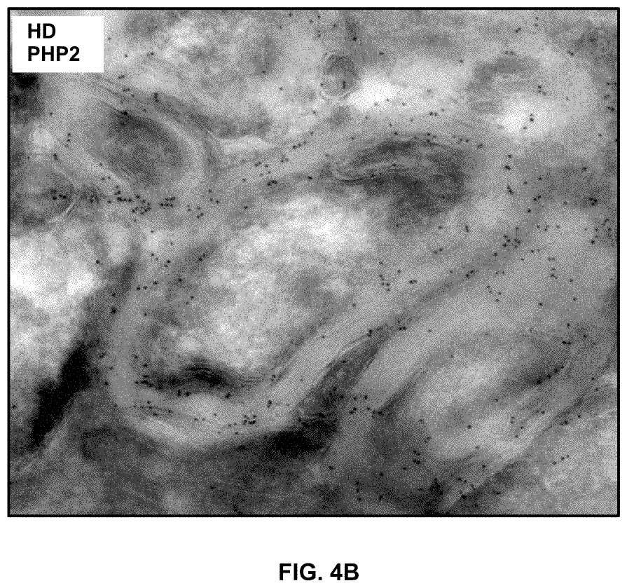

Without being limited by theory, it is contemplated that microbiota may affect myelination. Myelination is critical for the CNS function and may occur early prior to onset of symptoms in HD patients (Bartzokis et al., 2007). It is observed herein that mutant HTT oligomers accumulate into the myelin sheath, which may cross into oligodendrocytes and impair myelin production and myelin sheath function (FIG. 4). Accordingly, it is contemplated that microbiota may impact myelination.

GF mice have thicker myelin and elevated expression of myelin-related genes (Gacias et al., 2016, Hoban et al., 2016). Microbiota may also affect these events in HD mice. Thus, myelination in GF and conventional HD mice are compared electron microscopy. Levels of myelin expression are quantified by Western blots and QRT-qPCR, and it is expected that HIT aggregates are reduced in GF HD mice than in control HD mice.

The oligomerization of mutant HTT is a determinant of neurotoxicity and HD pathology in various models and serves a prominent biomarker in HD research (Bates et al., 2015). Based on findings in HD flies (FIGS. 1 and 2), levels mutant HTT aggregates in the ENS and are measured in GF HD mice in a confirmatory study, and it is expected that HTT aggregates are reduced in GF HD mice than in control HD mice.

Without being limited by theory, aggregation can be protective or neurotoxic based on the conformations formed (Arrasate et al., 2012). It is observed that microbial-mediated mutant HTT aggregation can coincide with the development of motor symptoms in HD flies (FIGS. 2B-D). Without being limited by theory, it is contemplated that if aggregation is reduced or delayed in GF HD mice in accordance with some embodiments herein, the severity of motor defect may also be affected. Without being limited by theory, a link between the intestinal microbiota, hippocampal neurogenesis, and cognitive tasks is contemplated. This link is based on the finding reported herein that the GF Rett mice, have enhanced hippocampal neurogenesis (See Examples 6, 17). Furthermore, approximately 15% of the CNS genes affected microbiota are related to myelination (Hoban et al., 2016).

### Example 5

### Effects of Rifaximin on the Progression of Rett Symptoms

Treatments with compounds and/or prebiotics/probiotics which regulate the homeostasis of intestinal microbiota can offer useful strategies to alter CNS symptoms such as those in Rett syndrome in accordance with some embodiments herein. Rifaximin is a gut-specific antibiotic, which is poorly absorbed in the circulation. In clinical studies, rifaximin decreases bacterial load, corrects leaky gut, and reduces the severity of several aberrant behaviors including anxiety, irritability, depression, motor symptoms, and cognitive impairment in Hepatic Encephalopathy (HE) patients (Bajaj et al., 2013, Kok et al., 2013). HE is a model of impaired microbiota-liver-brain axis (Bajaj, 2013). In a pilot study, rifaximin also improved motor functions in Parkinson's disease patients. Clinical trials are underway to test its effects in detail (accessible on the world wide web at clinicaltrials dot gov, Fasano et al., 2013).

Rifaximin has been tested in a Rett syndrome mouse model, which displays severe GI and metabolic abnormalities, motor symptoms, and cognitive impairment. Rifaximin was administered to the Rett mice as described in Example 19. Rifaximin eliminated the GI and metabolic symptoms of Rett mice (See, e.g., Example 14). Furthermore, Rifaximin-treated Rett mice are more active in the cage environment and build better nests (FIG. 5A). Nest building is indicative of rodents' well-being, daily activity, positive motivational state, and healthy brain functions (Jirkof, 2014). Rifaximin-treated Rett mice also perform better in climbing and wire-mesh hanging assays, and have significantly reduced clasping phenotype (FIGS. 5 B-D).

These assays, which are indicative of muscle strength, demonstrated that changing the Rett syndrome gut environment using rifaximin in accordance with sonic embodiments herein can influence the motor behavior in Rett syndrome, and improve motor function, as well as well-being, daily activity, positive motivational state, and healthy brain functions.

### Example 6

### Effects of Rifaximin on Hippocampal Neurogenesis and Cognitive Tasks in HD

It has been observed herein that rifaximin reduces the aggregation of mHDx1 in the Drosophila nervous system (Example 1, FIGS. 1A-D). Accordingly, it is contemplated that rifaximin can affect the pathology and behavior of HD mice. Additionally, Rett mice (as tested herein; see, e.g., Examples 5, 17) develop motor symptoms similar to R6/2 HD mice. Thus, it is contemplated that the effects of rifaximin can be tested on motor phenotypes of HD mice.

Accordingly, rifaximin is administered to R6/2 HD mice using the methods described in Example 19. HD offspring are treated with rifaximin after weaning for ˜4 weeks. Gut and brain sections of the vehicle and rifaximin treated animals are examined by aggregate-specific antibodies and quantified. Conditions for the delivery of rifaximin using Rett syndrome mice are described below.

Rotarod, beam crossing, and movement in open field are tested on vehicle and-HD-treated R6/2 HI) mice using methods described in Example 19. Effects of rifaximin on motor phenotypes of HD mice are observed.

Effects of rifaximin on nest building behavior of rifaximin-treated and vehicle-treated R6/2 HD mice are observed using the protocols described in Example 19. Effects of rifaximin on cognitive phenotypes of HD mice are observed.

Additionally, HD patients suffer from cognitive task and develop dementia (Paulsen, 2011). R6/2 Hf) mice display reduced neurogenesis and impaired learning and memory (Fedele, et al., 2011). It is reported herein that rifaximin promotes hippocampal neurogenesis and dendritic arborization in Rett syndrome mice (See Example 17 and FIGS. 3 and 5). These studies support the notion gut environment may influence hippocampal biochemistry and potentially impact cognition. Thus, effects of rifaximin on hippocampal neurogenesis in R6/2 mice are studied. Using immunohistochemistry with antibodies to markers of proliferating and developing neurons (1667, doublecortin) and BrdU labeling, the number newly of generated neurons are quantified and their fate is followed over time, including determinations of survival. Effects on rifaximin on hippocampal neurogenesis in HD mice are observed.

### Effects Rifaximin on Learning and Memory.

Behaviors such as novel object recognition and Barnes maze are performed on R6/2 HD mice treated with rifaximin, along with vehicle-treated controls to observe effects of rifaximin on cognitive tasks of HD mice.

### Identification of Microbial Communities Affected by Rifaximin.

Rifaximin promotes the proliferation of probiotic species including Bifidobacteria and Lactobacilli, which are indicative of healthy gut environment (Maccafferii et al., 2010, Ponziani et al., 2016). At a phylum level, it is reported herein that rifaximin enhances the growth of Tenericutes, which are reduced in Rett mice (FIG. 7).

Accordingly, 16S RNA sequencing is performed on gut microbiota of vehicle and rifaximin-treated WT and R6/2 HD mice, Microbial changes induced by rifaximin are observed.

Studies in this aim provide knowledge on whether changing the gut environment by rifaximin affects the progression of HD symptoms. Rifaximin reduces the aggregation of mutant HTT in HD flies (FIG. 1). Thus, without being limited by theory, it is contemplated herein that rifaximin may have similar effects in the ENS of HD mice. In view of data Rett syndrome mouse model described herein, it is also contemplated that rifaximin may reduce the motor symptoms of HD mice. There are proven beneficial effects of rifaximin on cognition and working memory have been reported in HE patients (Ahluwalia et al., 2014). In addition to inhibiting microbial growth, rifaximin also induces the expression and activates the detoxification signaling pathways such as pregnane X receptor (PXR), which maintains the integrity of intestinal epithelium and reduces the inflammatory conditions in the gut (Mencarelli et al., 2010). Rifaxirnin in clinical use for various microbial-mediated disorders and has minimal side effects.

Accordingly, it is contemplated that uses, methods, compositions, and product combinations comprising, consisting essentially of, or consisting of rifaximin can be useful in treating cognitive symptoms of HD.

### Example 7

### Roles of MeCP2 in Intestinal Biology and Gut Microbiota Homeostasis

The studies described in this Example were performed in MeCP2 (T158A) Rett mouse model, which carries a point mutation changing threonine 158 to alanine (T158A). T158A mutation prevents the binding of MeCP2 to DNA and increases its turnover. The line was produced by Dr. Zhaolan (Joe) Zhou's laboratory at the University of Pennsylvania and deposited in the Jackson's laboratory for the Rett community. T158A Rett mice phenotypes include developmental regression, motor dysfunction, stereotypies, breathing irregularities and learning and memory deficits (Goffin et al., 2011). The severity of symptoms are comparable to those displayed by MeCP2-null mice. Male mice develop symptoms at ˜2 months after birth and gradually die. Females on the other hand, become symptomatic when they are ˜8-9 months old. Interestingly, female Rett mice begin to gain weight at ˜4 months of age and progressively become obese. The cause of these metabolic changes remains unknown. However, intestinal dysbiosis may play a role and is further supported in the subsequent sections. Details for performing various assays are provided in the materials and methods section.

### Determine Whether MeCP2 Expression in the Intestinal Epithelium of Rett Mice Alters GI Physiology.

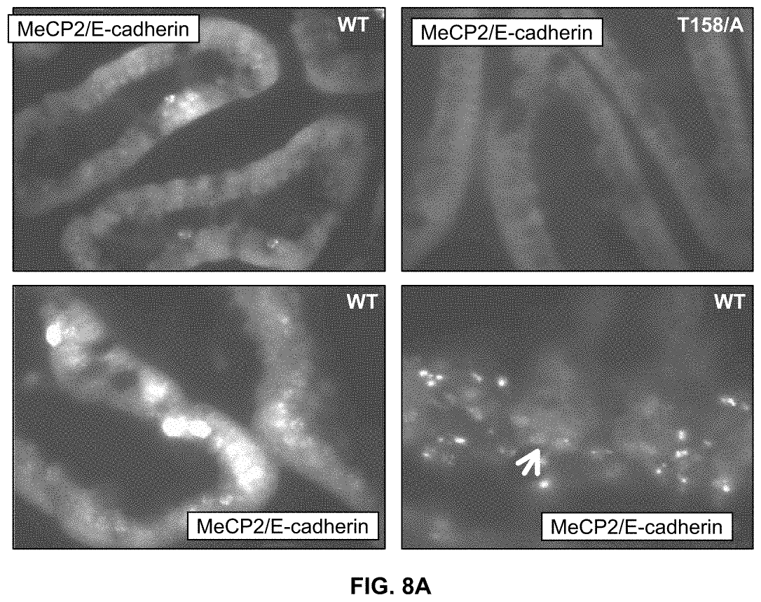

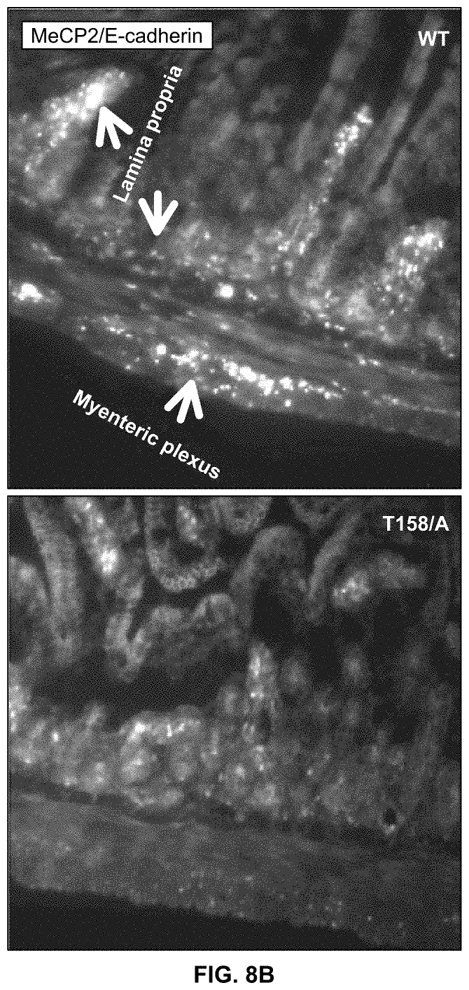

As one of the largest organs, the GI tract of adult humans is ˜22 feet long and has an area of 250 m2. The surface of the gut is protected by the intestinal epithelium, which serves many functions such as absorbing nutrients, producing growth factors and antimicrobial peptides, preventing colonization by harmful bacteria, and forming a barrier to prevent the leakage of gut contents into circulation. Conditions were established to detect MeCP2 in the intestinal epithelium by immunohistochemistry (FIGS. 8A-B). Notably, MeCP2 is expressed in the crypt cells, which are precursors for the regeneration of gut epithelium (FIG. 8A, arrow right bottom panel). MeCP2 is abundant in the myenteric plexus (enteric nervous system), and in the immune cells infiltrating the lamina propria (FIG. 8B, arrows top panel). It is observed that the intensity of MeCP2 staining is variable among villi(finger-like projections of the intestinal epithelium) of the small intestine suggesting that its levels might be regulated (FIG. 8A, top and bottom left panels). Bacterial LPS induces the expression and activity of MeCP2 in the CNS and alters the production of synapse-associated proteins implicated in cognition (Bie et al., 2013).

Without being limited by theory, these findings and the variable levels of MeCP2 in different regions of small intestine support the notion that MeCP2 may be a target of microbial signaling. Furthermore, these studies indicate a role of MeCP2 in gut physiology in the context of Rett syndrome.

### Example 8

### Anatomical Changes in the GI Tract of Rett Mice

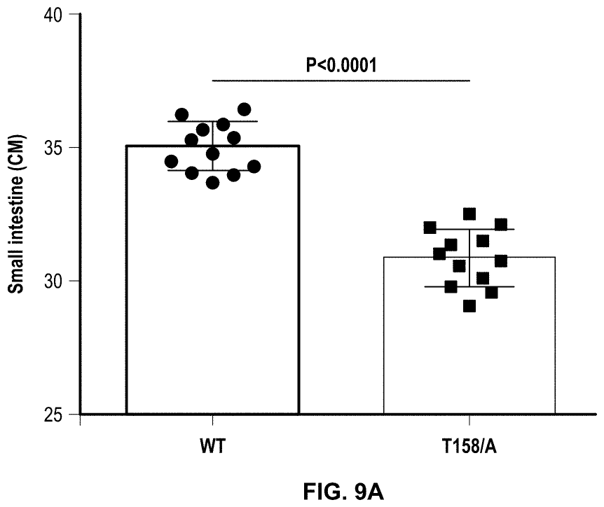

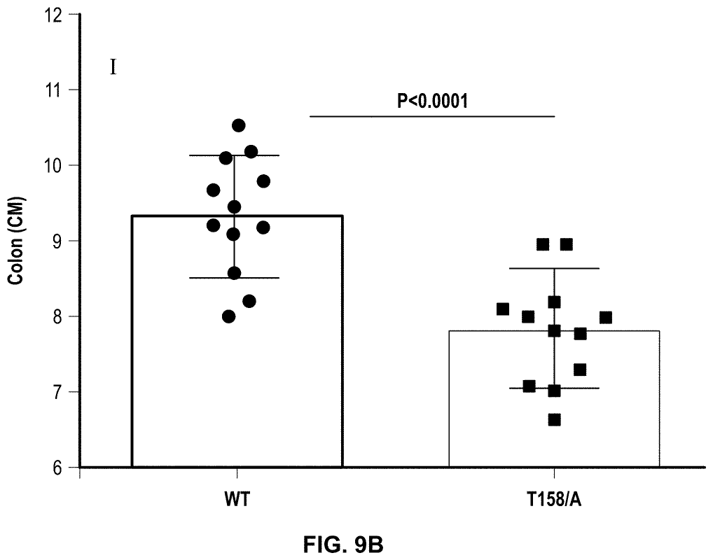

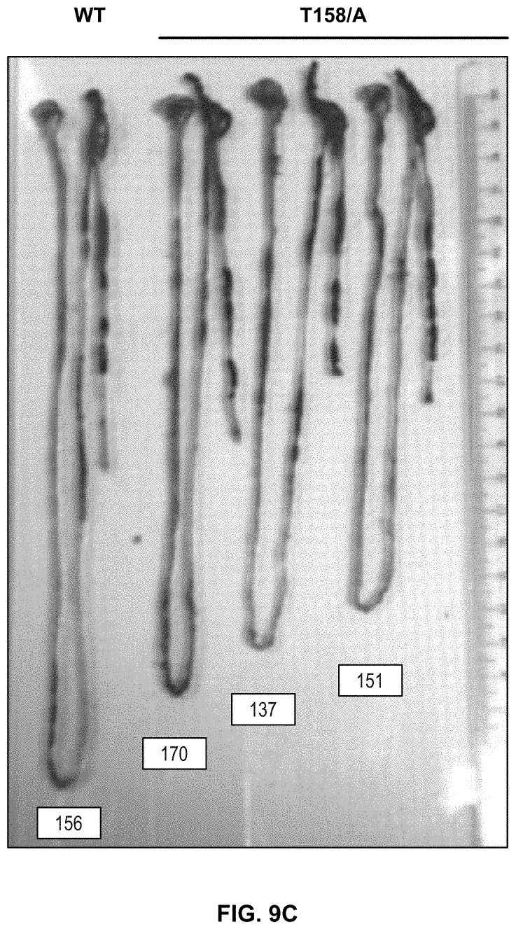

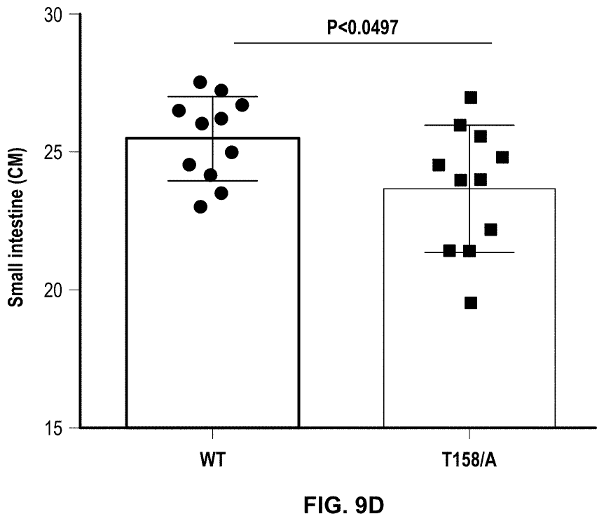

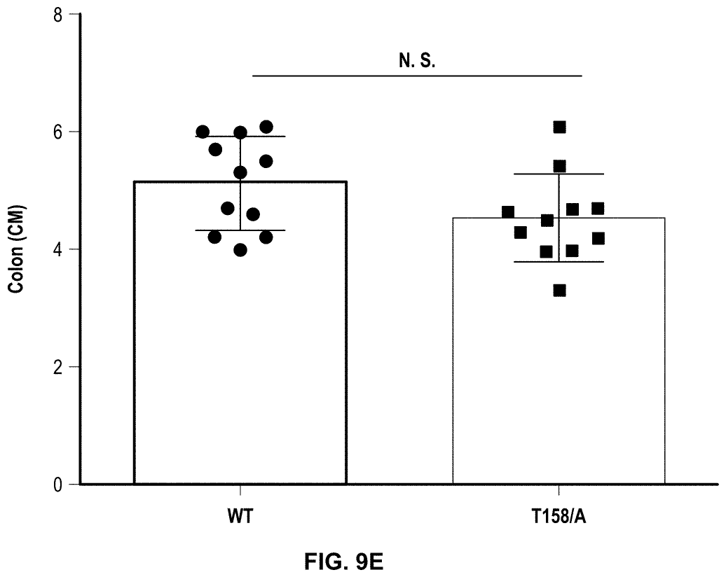

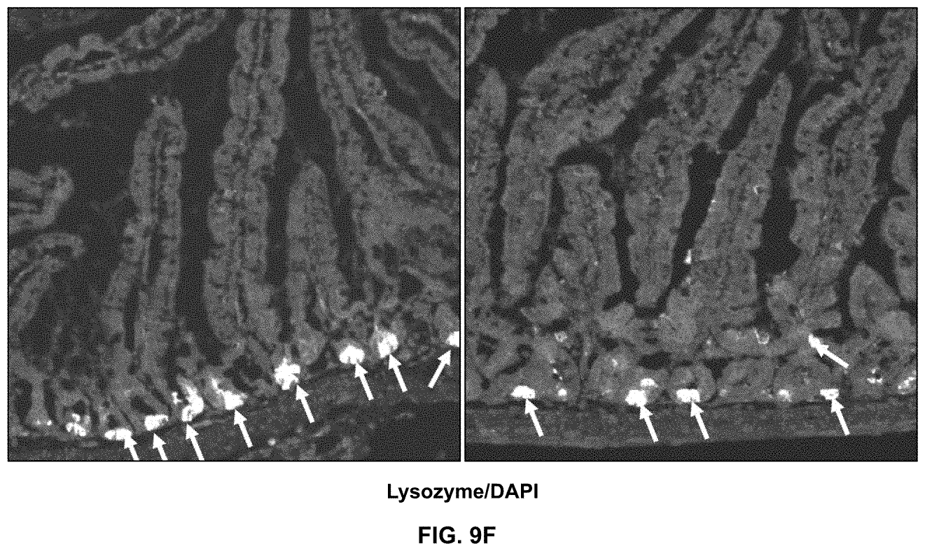

The GI tract of Rett mice were examined for gross anatomical changes. It is observed that the small intestine and colon of Rett mice are shorter than those in WT littermates (FIGS. 9A-C). These changes were independent of body weight or size. Interestingly, the gut length is not significantly different at weaning, suggesting that aging and/or environmental factors may contribute to abnormal gut development in Rett mice (FIGS. 9-E). Histologically, Rett mice display regional reduced number of paneth cells in the crypts (cellular structures that can contain Paneth cells, such as can be found in the intestinal epithelia), which are identified by staining with antibodies recognizing the antimicrobial peptide lysozyme (FIG. 9F). Paneth cells are involved in the continuous regeneration of intestinal epithelium and produce some of the most potent antimicrobial peptides for host-microbiota homeostasis (Clevers, et al., 2013a). Examination of mRNA isolated from the ileum portion of the small intestine suggests that the expression of antimicrobial such as defensins and lysozyme is significantly reduced in Rett mice (data not shown).

Without being limited by theory, it is hypothesized that the focal reduction of paneth cells may be the starting point where disruption in the gut-microbiota homeostasis allows the overgrowth of harmful bacteria, which may stray from their normal niches and spread to other sites in the GI tract. These foci of “disturbed neighborhoods” induced by the absence of MeCP2 may also recruit immune cells and further exacerbate the GI pathology by the release of excess inflammatory mediators. Without being limited by theory, expressing MeCP2 in the intestinal epithelium of Rett mice is contemplated in order to determine determine whether MeCP2 normalizes the gut-microbiota homeostasis and ameliorates some of the observed phenotypes.

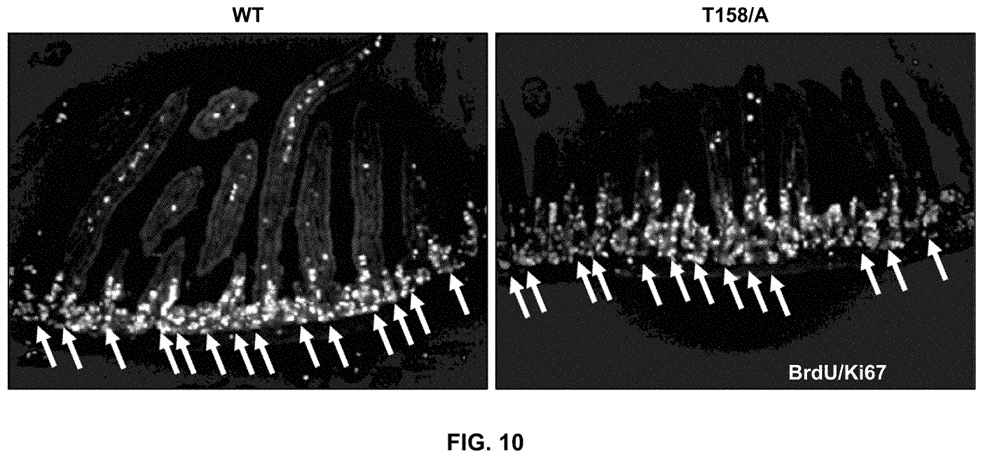

MeCP2Flox-STOP mice do not express MeCP2 but contain a floxed stop codon, which can be removed upon crossing with a line carrying Cre recombinase thus reactivating MeCP2 expression. The line was engineered by Dr. Adrian Bird's group at University of Edinburgh and deposited in the Jackson's laboratory for the Rett community (Guy et al., 2007), We previously used this line and successfully reactivated MeCP2 in the immune system by crossing with a vav-Cre line (Khoshnan et al., unpublished data). The MeCP2Flox-STOP heterozygous females can be crossed with WT villin-Cre male mice to reactivate MeCP2 selectively in the gut epithelium. Villin-Cre mice express Cre recombinase in the intestinal stem cell compartment and other cells involved the proper regeneration of the epithelium, and also in the post-mitotic epithelial lining of small and large intestine (Clevers, et al., 2013b). The newly generated line is referred to as MeCP2vil-Cre+. MeCP2 expression can be confirmed by immunohistochemistry using similar procedures as in FIG. 8. Once the line is established, the offspring are examined for changes in the gut length and pathology, paneth cell numbers, and intestinal regeneration. BrdU labeling is performed, and the development and maturation of intestinal epithelial cells (IECs) is monitored. Incorporation of BrdU in the crypt cells, amplifying progenitors, and in the IECs is determined by staining each progeny with cell-specific biomarkers. Data are evaluated by standard statistical software (Hsiao et al., 2013). Effects of MeCP2 on paneth cell numbers are observed. Additionally, the gut mRNA of these foxed mice for the levels of known antimicrobial peptides by RT-qPCR (Clevers, et al., 2013a). Without being limited by theory, these studies may identify novel functions for MeCP2 regulating the development of intestinal epithelium and the production of antimicrobial peptides. Conditions to examine BrdU labeling in the gut of 4 weeks old mice are as shown in FIG. 10.

### Investigate the Role MeCP2 in Gut-Microbiota Homeostasis.

Metagenomic sequencing of intestinal bacteria indicates that Rett mice have altered microbiota when compared to WT cohorts. The proportion of a major bacterial phylum Firmicutes is increased by ˜10%. Rett mice also have significantly less Tenericutes and Actinobacteria (FIG. 11A and 11B, marked). A genus within the Actinobacteria phylum is Bifidobacteria, which are prominently involved in the homeostasis of intestinal microbiota. Bifidobacteria are eliminated from the GI tract of Rett mice starting at ˜6 weeks of age, which precedes the appearance of Rett-associated symptoms (FIG. 11c, Goffin et al., 2011). The microbiota of 4 months-old female Rett mice were recently sequenced and dysbiosis was identified. Although there are differences in the microbiome of males and females, they share the common phenotype of having reduced levels of Bifidobacteria. These finding were confirmed with semi-quantitative PCR (FIG. 11D). It is relevant that the abundance of Bifidobacteria in the gut decreases with age and the loss is more prominent in patients with IBD, obesity, allergies, and some autistic patients (Rivière et al., 2016). Bifidobacteria are also among the first and most prominent species colonizing the gut of infants and their presence is a hallmark of a healthy environment producing metabolites for immune development and limiting the growth of pathogenic bacteria (Arboleya et al., 2016). Moreover, a recent report shows intestinal dysbiosis in Rett patients (Strati et al., 2016). The results reported herein support the notion that MeCP2 is prominently involved in the homeostasis of intestinal microbiota in mammals.

In view of the foregoing, the gut microbiome (collective bacterial DNA) is profiled in WT, parental MeCP2-null mice, and MeCP2 vil-Cre+ by 16S RNA sequencing to determine whether reactivation of intestinal MeCP2 normalizes dysbiosis. The bacterial DNA are sequenced from both males and females. As data presented herein suggest that dysbiosis in female Rett mice occurs at ˜4 months of age whereas in males it is apparent at ˜6 weeks after birth, similar time points are used for profiling the intestinal bacteria of newlines. Sequencing, compiling and computational analysis of data are carried out. Colonization by Bifidobacteria is also a useful assay to monitor changes in the microbiota longitudinally. Notably, Bifidobacteria support the propagation of other species in the gut especially those producing small molecules, which affect the immune system and brain functions (Arboleya et al., 2016). Thus, these experiments include identifying changes in Bifidobacteria.

### Example 10

### Roles of MeCP2 in Leaky Gut and Metabolic Defects

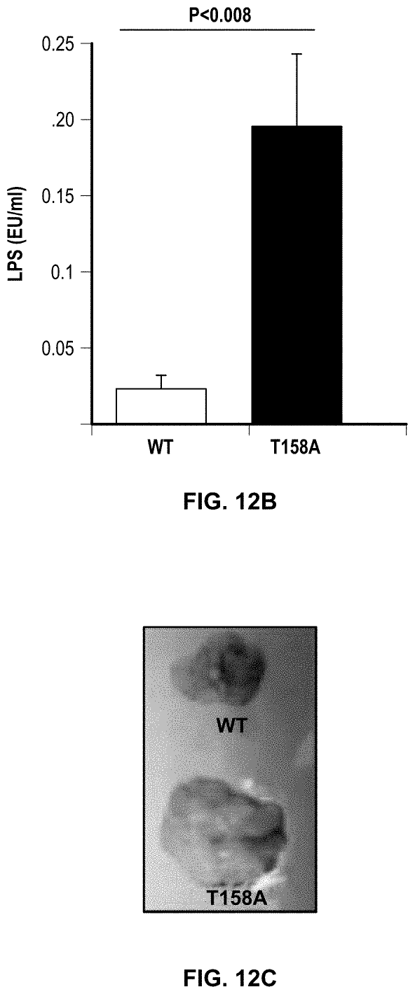

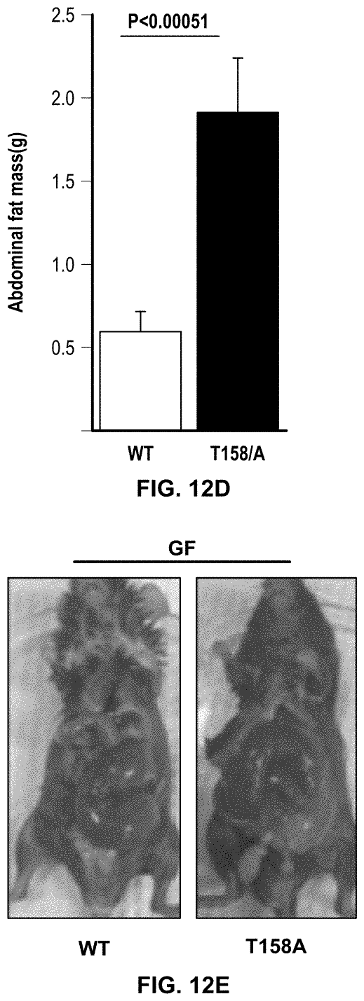

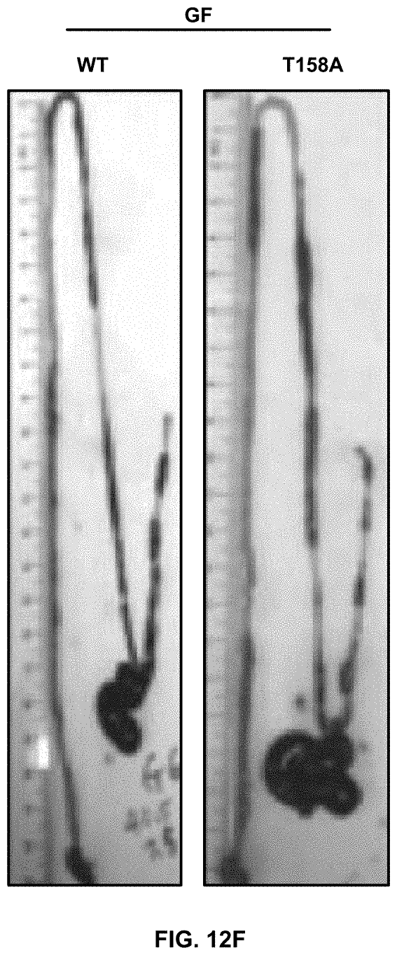

The integrity of intestinal epithelium is involved in the overall health and the proper gut-microbiota interactions. The GI tracts of Rett mice appear inflamed with some areas transparent and filled with air bubbles (FIG. 12A). Compared to WT, Rett mice also have elevated levels of LPS in the sera lending support to a “leaky gut” phenotype (FIG. 12B). LPS readily enters the CNS, which may cause neuroinflammation and disrupt many biological processes including hippocampal neurogenesis (Trotta et al., 2014). Moreover, Rett mice accumulate elevated levels of abdominal adipose tissue (FIGS. 5C-D). MeCP2 has previously been implicated in lipid metabolism where it regulates the expression of enzymes involved in the biogenesis of fatty acids (Kyle et al., 2016). It is notable that germ-free (GF) Rett mice do not have significant abdominal fat and the gut length appears normal. These findings suggest that the interactions between intestinal MeCP2 and microbiota may influence gut physiology and the associated metabolic pathways (FIGS. 12 E-F).

To determine whether MeCP2 regulates intestinal permeability, the levels of LPS in the sera of WT, Rett (MeCP2 Flox-STOP) and those expressing MeCP2 only in the intestine(MeCP2 vil-Cre) are quantified and compared. Leaky gut is also investigated by measuring the flow of fluorescent FITC-labeled dextran sulfate into the circulation. These protocols are as described in Hsiao et al., 2013. Also, the mice are examined for the accumulation of abdominal fat to determine roles for intestinal MeCP2 is in lipid metabolism in the gut. GF Rett mice are also examined for leaky gut. Without being limited by theory, these experiments establish a role for MeCP2 in gut physiology and how its absence in Rett may promote GI and metabolic anomalies.

### Example 11

### Examination of Rett Mice for Immune Dysfunction

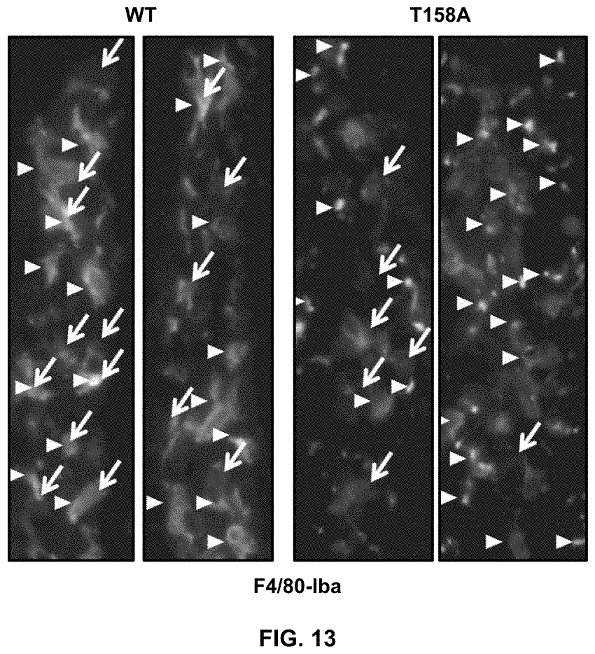

The homeostasis of intestinal microbiota is prominently involved in the proper development of the immune system. Rett mice display impaired immunity such as defects in the number and function of microglia and macrophages, and produce elevated levels of cytokine/chemokine (Cronk et al.,2015). It is relevant that loss of methylation-mediated epigenetic repression in the intestinal epithelium of zebrafish promotes cytokine-induced IBD (Marjoram et al., 2015). We speculate that the absence of MeCP2 in the intestinal epithelium impairs the gut-immune interactions. Observations herein suggest that the macrophages in the villi of Rett mice may be dysfunctional since they appear condensed and “apoptotic” (FIG. 13). This phenotype however, is sporadic suggesting that local “dysbiotic foci” may also alter the activity of gut immune cells regionally. Consistent with these findings, the numbers of intestinal macrophages are reduced in the symptomatic MeCP2-null mice (Cronk et al., 2015).

These results are repeated and quantified to further determine whether reactivation of MeCP2 could correct the “apoptotic “phenotype of the immune cells. Moreover, we macrophage/monocyte cells are isolated from the lamina propria of small intestine of WT, Rett (MeCP2Flox-STOP) and those expressing MeCP2 selectively in the intestine (MeCP2vil-Cre+),and perform multicolor flow cytometry to quantify and determine the phenotypes based on the expression of cell surface markers of activation. Since LPS regulates MeCP2 (Bie et al., 2013), the cytokine profiles of the intestinal monocytes/macrophages are also determined under resting and LPS-stimulated conditions using Luminex assays (See Hsaio et al., 2013 for protocol). Serum from all experimental animals is examined for cytokines and quantified similarly. Changes in the profiles are observed, and verified by RT-qPCR. These studies will confirm involvement of intestinal MeCP2 for the development and activity of innate immune cells.

### Example 12

### Behavioral Analysis of MeCP2vil-Cre+Mice

In mice with reactivation of MeCP2 in the intestinal epithelium as described herein, behavioral analysis is performed (as described in Example 19) to determine effects of normalizing the gut environment on any of the aberrant behaviors of Rett mice. For motor symptoms, rotarod, beam crossing, and clasping assays are used. Open field, light dark box, and marble burying are used for anxiety related disorders. Since breathing abnormalities are common in Rett, whole body plethysmography is used to determine whether expressing MeCP2 in the gut affects breathing potentially through lowering systemic inflammation and/or enhancing the production of protective microbial products. Effects of expressing MeCP2 in the gut on these parameters are observed.

### Example 13

### Evaluation of Intestinal Dysbiosis and MeCP2 in Rett Pathogenesis

GF mice are powerful tools for investigating the effects of microbiota on the host. We have developed a GF Rett mouse colony that can be useful forelucidating the impact of intestinal microbiota on the activity of MeCP2, determine the effects of gut environment on aberrant behaviors, and explore whether colonization with probiotics could lower the severity of any of the Rett symptoms.

Behavioral analysis of GF Rett mice. WT CIF mice have increased motor activity, reduced anxiety, and elevated levels of BDNF, which are in contrast to those reported in the conventional Rett mice and in patients (Lombardi, et al., 2015, Luczynski et al. 2016). Thus, aberrant behaviors of GFWT and Rett mice and cohorts raised under normal conditions can be investigated to determine if there are differences. By way of example, motor functions, repetitive behaviors, anxiety, and learning and memory can be examined in GF mice. At the end, breathing abnormalities can also be examined once a GF environment in no longer needed. Without being limited by theory, it is contemplated that these studies may link intestinal dysbiosis to the progression of CNS symptoms in Rett.

Transcriptional profiling of GF Rett mice. Understanding the molecular mechanisms of how the intestinal microbiota controls behavior in Rett may identify novel pathogenic pathways and therapeutic targets. If behavioral differences between the GF and conventional Rett mice are found, the mRNA can be sequenced and isolated from the intestine and brain tissues of all experimental groups and identify genes, which may be regulated by the microbiota-MeCP2 interactions. Selected targets of interest can be verified and quantified by RT-qPCR.

Intestinal colonization of GF Rett mice with probiotics. One promising probiotic is the commensal bacterium Bacteroides fragilis (B. fragilis), which colonizes the human gut (Mazmanian et al., 2005). Treatment with B. fragilis changes the intestinal microbiota favoring an anti-inflammatory environment and reduces the severity of autistic-like behaviors in an environmental mouse model of neurodevelopmental disorders (Hsiao et al., 2013). B. fragilis colonization is also protective in other disease models including multiple sclerosis and IBD (Chu et al., 2013). These findings have facilitated the clinical trials of B. fragilisin autistic children and patients with IBD.

To explore this therapy for Rett, GF Rett mice are colonized with B. fragilis and the effects on aberrant behaviors and pathology are examined as described herein (e.g., in Example 19). To validate B. fragilis for use in human patients, fecal materials of healthy human donors are enriched with B. fragilis and similarly colonize the GF Rett mice and examine the effects. Metagenornic 16S RNA sequencing and culturing are used to confirm colonization, bacterial load, and diversity. It is expected that B. fragilis will alleviate, decrease the likelihood, and/or delay the onset of symptoms associated with Rett syndrome.

### Example 14

### Effects of Rifaximin on Rett Mice

It was examined whether eliminating the intestinal microbiota of Rett mice alters GI pathology. Notably, treatment with a cocktail of antibiotics significantly increases the length of small intestine and reduced the accumulation of abdominal fat (FIG. 14A, additional data not shown). However, some of the antibiotics in the cocktail can be toxic, which complicates data analysis and further evaluation. Accordingly, it was studied whether clinically useful antibiotics may have similar effects. Rifaximin is a gut-specific antibiotic, which is safe and well tolerated by humans (Bajaj, 2013). In clinical studies, rifaximin decreases bacterial load, corrects leaky gut, and reduces the severity of several aberrant behaviors including anxiety, irritability, depression, motor symptoms, and cognitive impairment in Hepatic Encephalopathy (HE) patients (Bajaj et al., 2013, Kok et al., 2013). HE is a model of impaired gut-liver-brain axis with strong links to intestinal dysbiosis (Bajaj, 2013). In a pilot study, Rifaximin also improved motor functions in Parkinson's disease patients. Clinical trials are underway to test its effects in detail (accessible on the world wide web at clinicaltrials dot gov, Fasano et al., 2013).

It was observed that treating Rett mice after weaning with rifaximin for 6 weeks selectively elongates the colon and prevents intestinal obesity (FIGS. 14 B-C, data not shown). Female Rett mice also become obese and accumulate excess adipose tissue but not when treated with rifaximin prior to the development of these metabolic changes (FIGS. 14. D-F). Although weight gain may not be a concern in some Rett patients, these results suggest that the absence of MeCP2induces microbial-induced metabolic changes, which could be normalized by modifying microbiota. A subset of Rett patients however, gains weight and have abnormal lipid metabolism (Kyle et al., 2016).

These data show that treating Rett syndrome mice with the clinically useful antibiotic rifaximin significantly improves intestinal morphology and reduces the accumulation of abdominal fat.

### Example 15

### Effects of Rifaximin on Astrocyte and Microglia in Rett

Astrocytes are abnormal in Rett mice and reactivation of MeCP2 in the astrocytes corrects aberrant behaviors including locomotion and anxiety, increases life span, and normalizes breathing abnormalities (Lioy et al., 2011).

It was observed that brain sections from the cortical regions of some of the rifaximin-treated Rett mice have reduced staining for biomarkers such as glial fibrillary acidic protein (GFAP) and glutamine synthetase (GS) when compared to vehicle-treated cohorts (FIG. 15A). The finding can be re-confirmed and quantified for statistical analysis. Elevation of GFAP and GS is linked to astrocytes activation and inflammation. Astrocytes are recognized as therapeutic targets for neuroinflammation, neurodegeneration, and neuropsychiatric disorders. Subsets of gut microbes produce molecules, which reach the CNS, lower astrocytes activation, and subsequently reduce the production of inflammatory cytokines (Rothhamrner et al., 2016). Gut bacteria also greatly influence the development of microglia, which are the CNS immune cells (Erny et al., 2015). The brain sections of Rett mice were stained with microglial biomarkers and found that the numbers were reduced in some of the rifaximin-treated animals (FIG. 15B).

Thus, these data show rifaximin-treatment of Rett reduced staining for biomarkers linked to astrocytes activation and inflammation.

In these data, rifaximin was delivered in the drinking water, which might have resulted in the uptake of different dosage by each animal, so additional studies can be used for generating dosage curves. For example, animals can be gavaged with known amounts of rifaximin to ensure equal delivery. Astrocytes and microglia can be isolated from vehicle and rifaximin-treated mice and mRNA sequencing can be performed to identify gene products, which are regulated by rifaximin treatment.

### Example 16

### Effects of Rifaximin on Rett Behaviors

Rifaximin-treated Rett mice are more active in the home cage environment and build better nests (FIG. 16A). Nest building is indicative of rodents' wellbeing, daily activity, positive motivational state, and healthy brain functions (Jirkof, 2014). Without being limited by theory, it is contemplated that Rifaxitnin may also help with the motor symptoms of Rett mice since treated animals make more attempts to climb a wire-meshed cylinder, display enhanced muscle strength, and have reduced clasping phenotype, which is a sign of dystonia and loss of muscle coordination (FIGS. 16B-D). It is noted that rifaximin ameliorates the Parkinsonian symptoms of HE and PD patients (Fasano et al., 2013, Kok et al., 2013). This is consistent with the improved mobility in Rett mice observed herein. The data herein can be confirmed with additional behavioral tests, including rotarod, beam crossing, and open field. These studies can confirm benefits of rifaximin therapy on the CNS pathology and/or aberrant behaviors in Rett syndrome. Optionally, the intestinal bacteria that are affected by rifaximin can be identified as described in part of Example 17.

### Example 17

### Examine the Links Between Intestinal Microbiota and Hippocampal Neurogenesis in Rett

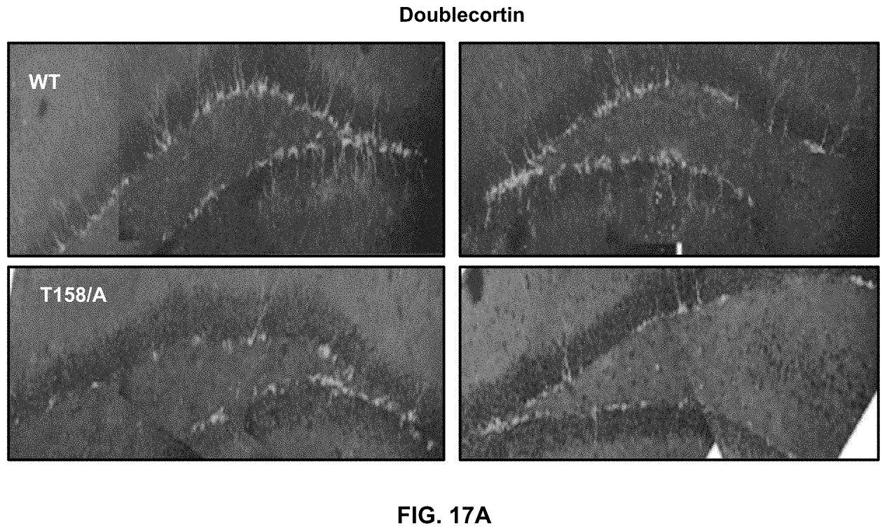

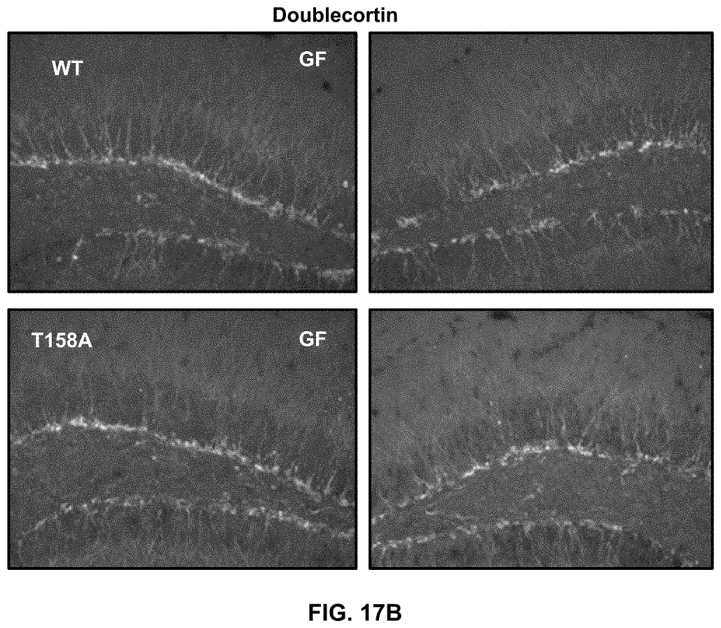

A common feature of Rett syndrome is intellectual disability. Defects in hippocampal neurogenesis is linked to cognitive impairment in Rett syndrome (Ra.mocki et al., 2008). Adult Rett mice display reduced hippocampal neurogenesis (FIG. 17A, bottom panels), which may impair cognition and induce anxiety and depression. Neurogenesis is normal in young conventional Rett mice. Moreover, GF Rett mice do not show any reduction in the number of newly generated hippocampal neurons (FIG. 17B). The findings can be further confirm and quantified these using BrdU incorporation. WT GF mice appear to have increased hippocampal neurogenesis (Ogbonna.ya et al., 2015), which may explain the results observed in GF Rett mice. Without being limited by theory, deep brain stimulation promotes hippocampal neurogenesis and enhances learning and memory in Rett mice suggesting that cognitive defects in Rett may be treatable by changing the CNS environment (Hao et al., 2015). Without being limited by theory, it is contemplated that manipulation of the gut physiology in accordance with some embodiments herein can induce hippocampal neurogenesis and enhance cognition in Rett mice.

### Determine the Effects of Microbiata on Neurogenesis in Rett Mice.

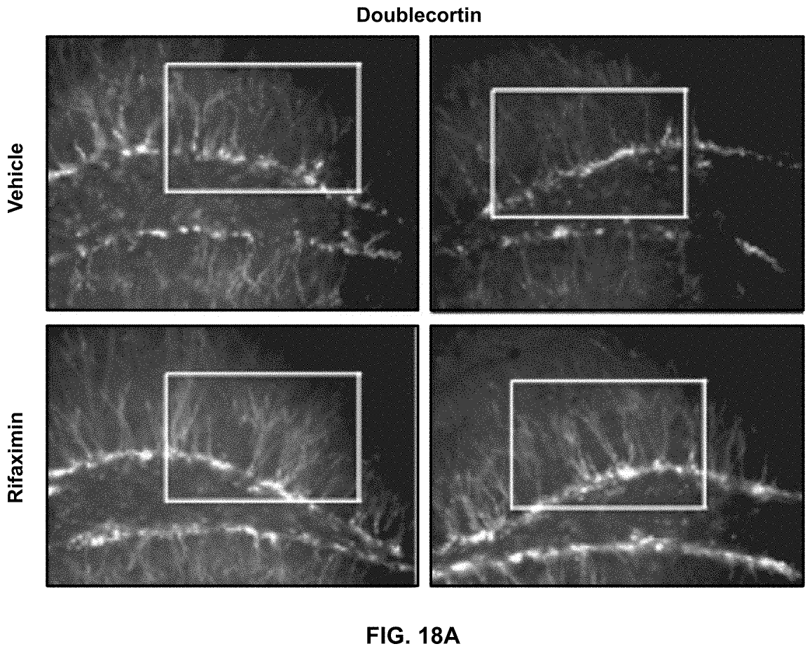

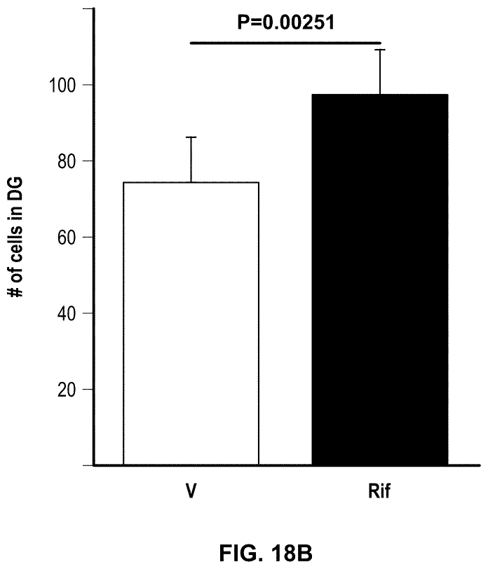

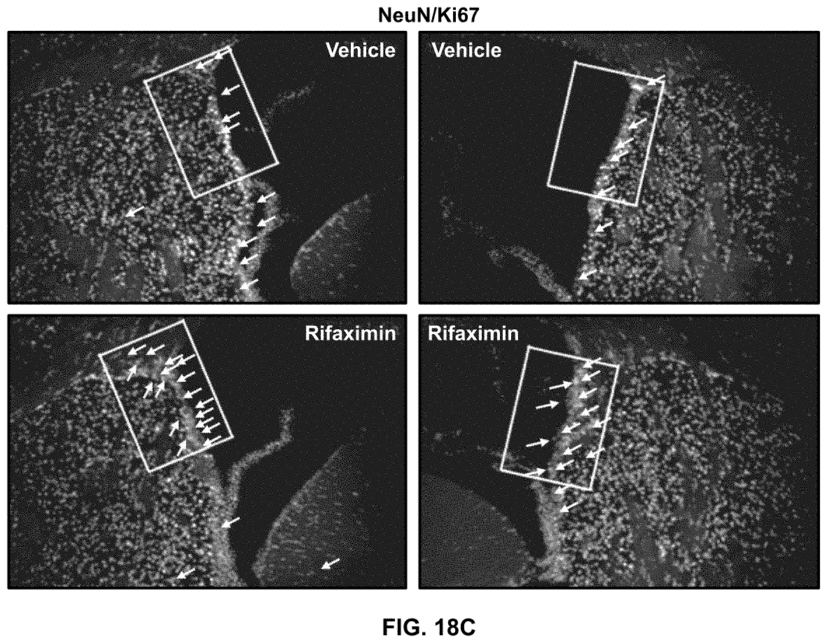

Since rifaximin improves the cognitive tasks of HE patients (Ahluwalia et al., 2014), it was tested whether rifaximin can affect neurogenesis in Rett mice. Data indicate that compared to vehicle, rifaximin-treated Rett mice display enhanced hippocampal neurogenesis and dendritic arborization (FIGS. 18A-B).

Enhanced proliferation of cells in the subventricular zone (SVZ), which is another niche for CNS neurogenesis, was also observed in rifaximin-treated mice (FIG. 18C). Without being limited by theory, these data indicate that rifaximin may have broad effects on the neurochemistry of Rett mice.

These studies indicate that changing the gut environment in accordance with some embodiments herein can modify the brains of Rett mice. Using BrdU labeling, it can further be determined whether the newly generated neurons survive over time and integrate into brain circuits.

### Identification of Hippocampal Gene Targets Influenced by Rifaximin.

GF Rett mice do not show any sign of impaired neurogenesis (FIG. 17B).

Accordingly, without being limited by theory, it is contemplated that the levels of selected genes can be similar between the conventional rifaximin-treated and GF Rett mice. Studies of these genes may identify hippocampal neurogenic pathways, which are regulated by the MeCP2-microbiota interactions.

Effects of rifaximin on learning and memory are determined. Behaviors such as novel object recognition and Barnes Maze are performed to determined effects of rifaximin treatment on the cognitive tasks of Rett mice. GF WT and Rett mice are also tested for these behaviors the results are compared with the rifaxitnin-treated cohorts. Impacts of intestinal microbiota and rifaximin treatment on cognitive tasks in Rett mice are observed. It is expected that learning and memory are improved in the rifaximin-treated Rett mice.

Additionally, for any observed effects of rifaximin on neurogenesis the molecular targets involved are identified. mRNA sequencing on the hippocampi of vehicle- and rifaximin-treated WT and Rett cohorts is performed. Results are verified by RT-qPCR, and are further verified in GF Rett mice.

Thus, rifaximin is contemplated as a candidate for the treatment of intellectual disability in accordance with some embodiments herien.

### Example 18

### Identification of Microbial Communities Affected by Rifaximin

Treatment of human microbiota with rifaximin promotes the proliferation of probiotic species including Bifidobacteria, which are reduced in Rett mice (Maccafferii et al., 2010, FIG. 4C). Surprisingly, rifaximin did not increase the abundance of Bifidobacteria in the GI tract of Rett mice suggesting that its protective effect may be due to changes in the composition of other bacteria. Interestingly, rifaximin increased abundance of the phylum Tenericutes, which is also reduced in Rett mice (FIGS. 7, 11, and 19). Less is known about the role of Tenericutes in human health. In animals however, the presence of Tenericutes correlates with digestion of crude fiber (Niu et al., 2015). Regardless, these data suggest that rifaximin alters the composition of microbiota, which may favor colonization by neurogenic microorganisms.

For further study, 16S RNA sequencing of vehicle and rifaximin treated WT and Rett mice is performed. It is expected that these experiments will identify microbial changes induced by rifaximin in the presence and absence of MeCP2. For any cultivable bacterial species that are enriched, the top candidate can be used for colonization of Rett mice affects hippocampal neurogenesis and cognition related behaviors.

Without being limited by theory, it is further contemplated that rifaximin may reduce the growth specific bacterial species, which may impair neurogenesis. The role of such species can be further confirmed through colonization in GF Rett mice. Reduction of the number of newly generated neurons in the hippocampus, examination by immunohistochemistry, and behavioral analysis can identify a role of such bacterial species in impairing neurogenesis.

### Example 19

### Materials and Methods

### Animal Models

All animal experiments are performed in accordance with the Institutional Animal Care and Use Committee and Caltech Animal Guidelines. MeCP2-null (MeCP2Flox-stop) and MeCP2 (T158A) are used for most experiments. MeCP2vill-cre+ are generated by crossing of MeCP2Flox-stop and villin-Cre. To reduce cost and reach a conclusion quickly, the male will initially be examined since males produce robust phenotypes within a short time. If the results of any experiment are positive, they can be validated in females. It is estimated that for many behavioral analyses ˜15-20 mice are required for each experimental group to achieve differences with a P value of <0.1-0.5 significance. This generally requires about 5-6 pregnant females for each group. For histological and biochemical examination, and gene studies, about 6 mice are used for each group. The institutional regulatory reviews for animal use for this project are completed, and the Caltech IACUC has approved the experimental procedures required for completion of the proposed studies (protocol #1726).

### Derivation and Examination of GF Rett Mice.

GF Rett mice (T158A) are generated by procedures established in the gnotobiotic center at Caltech. Mice will routinely be tested for the presence of microbes by culturing under aerobic and anaerobic conditions and PCR. This colony is established and is currently being expanded for various experiments.

### Rifaximin Treatment

Rifaximin treatment is generally initiated when the animals are ˜4-5 weeks old and continue treatment for 6 weeks. This is based on observations that male mice start displaying symptoms when they are ˜10 weeks old. For the presented preliminary results, rifaximin was provided in the drinking water, which is non-invasive. If the variability in the outcome is large, the animals will be gavaged to ensure that each animal receives equal amounts of rifaximin (Hsiao et al., 2013).

### Histology, Immunohistochemistry, and Immunofluorescence.

To test whether Rett mice display GI defects, they will be examined for gross anatomy and measure in length as presented in FIG. 9. They will also be examined for the differentiation of intestinal stem cells in various Rett models since MeCP2 may influence their production. Intestinal stem cells self-renew and differentiate into endocrine cells, enterocytes, goblet cells, and Paneth cells (Yin et al., 2014). Each cell type is identified by the expression of specific markers, which could be identified by immunochemistry. Intestinal stem cells express markers such as Lgr5/GPR49. Enterocytes have many distinct markers such as E-cadherin, alkaline phosphatase and lectin binding proteins (FIG. 8). Goblet cells could be identified by selective expression of mucin (MUC2), and paneth cells are identified by the specific expression of markers such as and lysozyme (Yin et al., 2014, FIG. 9F). Antibodies to these markers are commercially available and have been used in similar studies. Antibodies selective for each cell marker will be used to indicate whether the presence or absence of MeCP2 will affect the differentiation of intestinal stem cells and alter the ratio of different gut cells in the intestine.

### BrdU Labeling.

Without being limited by theory, it is expected that abnormal proliferation of intestinal stem cells may be responsible for shorter gut length since MeCP2 is expressed in the intestinal stern cells and those supporting the regeneration of intestinal epithelium (FIG. 8). To test this further, intestinal stern cells of adult WT and Rett mice are labeled by injecting BrdU (50 ug/gram body weight), and following the incorporation over 3-4 day, which is the time it takes to renew the intestinal epithelium. Mice are sacrificed at various time points post-injection, fixed, and examined by immunohistochemistry. A representative picture for BrdU labeling of intestinal epithelium is shown in FIG. 10.

### Examination of Leaky Gut.

To test whether Rett mice display leaky gut, male and female WT and Rett mice are fasted overnight and orally fed (via gavage) the following day with FITC-dextran (Sigma, 4K). In previous studies 0.6 mg/kg was used (Hsiao et al., 2013). Similar procedures and concentrations are used in the present experiment. Plasma samples are collected 4 hr post-gavage and the intensity of fluorescence are measured using a spectrophotometer (excitation 485 nm/emission, 535 nm). For each experiment, six symptomatic mice for males and 6 for females plus aged-matched WT are used. Since male Rett mice develop symptoms earlier, the experiments are be done at different ages (˜3 months for males and ˜8 months for females). If leaky gut is present in Rett mice, a cohort of pre-symptomatic animals will be examined to determine whether leaky gut precedes the development of symptoms. A positive result will indicate that leaky gut may be a developmental defect, which may contribute to some of the Rett-associated symptoms. Leaky gut is measured by quantifying the levels of LPS as presented in FIG. 128. It is expected that Rett mice may display leaky gut.

### Isolation of Macrophages, Microglia and Astrocytes.

Intestinal macrophages are enriched according to published protocols (Harusato et al., 2016). Briefly the small intestine of animals are dissected by standard procedures and digested in collagenase solution. Single cell suspensions are isolated and stained with monocytes/macrophage cell surface markers and quantified by flow cytometry. Caltech's Flow Facilities are used for these experiments. Similar procedures are used to isolate microglia and astrocytes from the CNS. Commercially available kits from Miltenyl Biotec are utilized to enrich for each cell type (San Diego, Calif.).

### Analysis of Serum Cytokines and Chemokines.

The gut macrophages/monocytes of WT and Rett mice (pre-symptomatic and symptomatic) are examined for the production of inflammatory cytokines in response to LPS stimulation, using Luminex assays, which are designed to simultaneously measure multiple cytokines and chemokines. Procedures for these assays are established (Hsiao et al., 2013). To get statistical significance, 6 mice are used each for WT and mutant per run. The expression of altered targets are confirmed by RTqPCR analysis of mRNA isolated from resting and LPS-stimulated macrophages. It is expected that altered targets will be observed.

### Microbiota Sequencing.

To better understand whether Rett mice display intestinal dysbiosis, the microbial profiles of gut samples are determined by 16S RNA sequencing (FIG. 11). To gain detailed insight into the gut microbiome of Rett mice, we will sequence fecal samples from both males and females. Six samples from each group (WT and Rett mice), and ages 3 weeks, 2 months and 4 months are collected and DNA is extracted for sequencing. These procedures provide genus and species identification for isolates with ˜90% accuracy. Caltech Genomic Facility is used for sequencing and bioinformatics analysis mRNA sequencing. For mRNA expression studies, intestinal fragments or brain regions are isolated by standard procedures. Total RNA is be extracted by TriZol and was further purified by RNA purification columns. The Caltech Genomic Facility is used to perform mRNA sequencing, perform computational analysis of the data and identifying the targets, which may be affected by microbiota. Selected targets are validated by RT-qPCR using standard procedures. It is expected that differently expressed microbial gut profiles are observed in Rett mice.

### Biotherapy of Rett Mice.

B. fragilis treatment is protective in environmental and genetic mouse models of autism (Hsiao et al., 2013). To explore its effect in GF Rett mice, 1×1010 CFU (colony forming units) of freshly grown B. fragilis are suspended in 1.5% sodium bicarbonate, mixed with 4 ml sugar-free applesauce and spread over four standard food pellets. We find that 42% of B. fragilis colony forming units are recovered from the applesauce inoculum at 48 hr after administration, suggesting that both viable and nonviable B. fragilis is ingested during the treatment (Hsiao et al., 2013). For vehicle treatment, animals are fed 1.5% sodium bicarbonate in applesauce over food pellets. Previous studies indicate that applesauce and pellets are completely consumed by mice very rapidly when each animal is single housed (thus, we will overcome cage effects). This treatment is repeated 3× in 1 week to ensure sufficient uptake of B. fragilis. Both asymptomatic and symptomatic male and female mice are treated. Each experiment uses 6 mice each (WT and Rett, pre-symptomatic, symptomatic). Six weeks post-treatment, behavioral analysis is performed and other phenotypes are tested, such as weight gain and effects on life span to analyze whether B. fragilis has any protective effects. For therapeutic purposes, we will also enrich the fecal materials of healthy human donors with B. fragilis and examine similarly. It is expected that these studies will support the notion that changes in gut microbes may contribute to the progression of Rett symptoms, and that restoring microbiome dysbiosis with a probiotic may be a potential therapy. It is expected that B. fragilis treatment will ameliorate, reduce the likelihood, and/or prevent symptoms associated with Rett in accordance with some embodiments herein.

### Behavioral Analysis of Treated Mice.

### Rotarod.

Mice are trained for two consecutive days before the initial test. The latency to fall from a rotarod beginning at 5 rpm and accelerating to 40 rpm over 240 s is scored. Mice are allowed to stay on the rotarod for a maximum of 300 s. Two trials are performed per training day with a 10 min intertrial interval (ITI). Two trials are performed separated by a 10 min

### Beam Crossing.

The time to cross the center 80 cm of a 1 m beam is scored. We use square beams of different widths (28, 12, and 6 mm). The beams are mounted on poles (50 cm above the tabletop) with a bright light at the starting end and a dark box containing the animal's home cage nest material at the far end. A nylon hammock 7.5 cm above the tabletop is used to prevent injury to mice falling from the beam. Mice are placed at the end of the beam with the bright light and the time from when the entire body of the mouse enters center 80 cm portion of the rod to the time that the nose of the mouse exits the center is measured using an infrared interrupt sensor. Data are analyzed as described by Southwell et al., 2009. Both rotarod and beam crossing will evaluate motor symptoms.

### Clasping.

Hind limb clasping is a marker of disease progression in Rett syndrome and other diseases with motor defects. In this assay animals were suspended by the tail ˜30 cm above the tabletop for 1 min and recorded with a video camera. No clasping is scored as 0, periodic clasping and extension is scored as 1, and full hind limb clasping was scored with occasional extension is scored as 2. Severe clasping is scored as 3.

### Open Field.

Mice are placed in the lower left corner of a 50×50 cm open white Plexiglas box with 16 cm sides in a room brightly lit by fluorescent ceiling lights. Open field activity is recorded for 10 min by a ceiling mounted video camera. Center entries and time spent in the center are scored. Center entries will examine anxiety-like behaviors. Data are analyzed as reported (Hsiao et al., 2013).

Marble burying.

This is a test for repetitive and stereotyped behaviors, which is a hallmark of autism and Rett. In this test, mice are placed into a testing cage containing marbles placed on the surface of the bedding and the number of marbles buried (>50% marble covered by bedding material) in a 10 minute period is recorded (Malkova et al., 2012).

### Plethysmographic Measurements.

Whole body plethysmography are used to measure breathing irregularities in Rett mice. This phenotype is common in Rett patients (Lombardi et al., 2015). An accumulating body of literature supports the potential importance of vagus nerve in the regulation of breathing. We predict that intestinal dysbiosis in Rett impairs the vagus nerve activity and exacerbate the breathing abnormalities. Moreover, we predict treatment with rifaximin or B. fragilis may correct some of the vagus nerve defects in Rett mice and subsequently ameliorate some of the breathing defects. The Buxco Fine Pointe Unrestrained Whole Body Plethysmograph system from Data Sciences International (DSI; St. Paul, Minn.) are used for these measurements.

### Novel Object Recognition.

The novel object recognition is a useful test to examine the effects of rifaximin or probiotics on learning and memory in Rett mice. We previously used this test in an environmental mouse model of autism (Hsiao et al., 2013). Rodents in general spend more time exploring a novel object than a familiar one. The ability to choose the novel object is indicative of learning and recognition memory. The novel object recognition task is conducted in an open field arena with two different kinds of objects. Both objects are generally consistent in height and volume, but are different in shape and appearance. During habituation, the animals are allowed to explore an empty arena. Twenty-four hours after habituation, the animals are exposed to the familiar arena with two identical objects placed at an equal distance. The next day, the mice are allowed to explore the open field in the presence of the familiar object and a novel object to test long-term recognition memory. The time spent exploring each object and the discrimination index percentage are recorded. This test is used, among other experiments, to assess the cognitive ability of vehicle- and rifaximin-treated Rett mice as well as GF cohorts and quantify and compare the results.

### Barnes Maze.

The Barnes maze is highly suitable for analyzing the effects of probiotics or rifaximin on hippocampus dependent spatial memory task. In this test mice learn the position of a hole to escape the brightly lit open surface of the maze. The established standard protocols are followed for this test (Dawood et al., 2004). Briefly, the animal is placed in a brightly lit environment, on the top of the Barnes maze, which consists of a large round open platform provided with a fixed number of peripheral holes. In such an open environment, mice naturally seek a dark enclosed surrounding, which is provided in the form of a dark box (goal box) under one of the round holes around the perimeter of the platform. Mice are trained to identify the escape hole repeatedly. This test is used to assess the cognitive ability of vehicle- and rifaximin-treated. Rett mice as well as GF cohorts and quantify and compare the results. For data acquisition the amount of time required for the animal to locate the goal box using visuo-spatial cues surrounding the maze periphery are measured. The number of errors the mice make are recorded to find the escape hole. All training, experimental setups and data acquisitions are according Dawood et al., (2004).

### Example 20

### Neurotransmitter Levels and Expression of Cholinergic Genes in Rett Mice

Levels of cholinergic genes were measured in gut samples Rett mice and wild-type (non-Rett) control mice by quantitative reverse-transcriptase PCR. Compared to wild-type (non-Rett) control mice, Rett mice exhibited reduced levels of cholinergic genes (See FIG. 20A). Cholinergic genes that were reduced in Rett mice included Chrna2, Chrna7, Chrb4, Chrm1, Slc5a7, Char, Ache, and Slc18a3.

Serum levels of neurotransmitters were measured in Rett mice and wild-type (non-Rett) control mice by gas chromatography mass spectrometry. Levels of choline, tyrosine, dopamine, and epinephrine were increased in Rett mice compared to wild-type (non-Rett) controls. Levels of serotonin (5-HT) were reduced in Rett mice compared to wild-type (non-Rett) controls. See FIG. 20B.

### Example 21

### Reactivation of MeCP2 in the Rett Gut

It is contemplated that MeCP2 expression in the gut of Rett mice may ameliorate GI pathology and normalize the gut-microbiota homoeostasis. To study this, a villin-Cre mouse line is crossed with fluxed MeCP2Flox-STOP mice in order to reactivate MeCP2 expression selectively in the intestinal epithelium. Offspring of the engineered line are examined for gut pathology including barrier permeability, metabolic changes, dysbiosis, and aberrant behaviors found in MeCP2-null mice. It is expected that MeCP2 is important for gut-microbiota interactions and may link the gut pathology to the progression of CNS pathology in Rett syndrome.

### Example 22

### Behavioral Analysis is Performed on GF Rett Mice Colonized with B. fragilis and/or Rifaximi n

It is contemplated that intestinal dysbiosis may be a modifier of Rett pathogenesis. Rett patients and mouse models of Rett have altered intestinal microbiota. To connect dysbiosis to CNS symptoms, behavioral analysis is performed on GF Rett mice and compared to the aberrant behaviors displayed by the conventionally raised cohorts. If differences are detected, the mRNA extracted from the intestine and the brain of all experimental groups can be sequenced in order to identify potential gene targets and pathways, which may be regulated by the MeCP2-microbiota interactions. To test GI-based therapies, GE Rett mice are colonized with a probiotic Bacteroides fragilis alone or incorporated into the fecal materials of healthy human donors. It is contemplated that Bacteroides fragilis ameliorates some of the GI and/or CNS pathology and reduces the severity of aberrant behaviors.

Moreover, modification of the intestinal microbiota of Rett mice with rifaximin can be studied. Rifaximin is used to treat IBD and patients with gut-brain disorders. It is expected that the rifaximin treatment may normalize dysbiosis and decrease the GI and CNS symptoms.

### Example 23

### Survival of Newly Generated Neurons Following Rifaximin Treatment in Rett

Without being limited by theory, links between intestinal mnicrobiota and hippocampal neurogenesis in Rett are contemplated. Intellectual disability, which can involve defects in the hippocampal circuits, is prevalent in Rett patients. It is reported herein that rifaximin treatment promotes hippocampal neurogenesis and dendritic arborization in Rett mice. Survival of the newly generated neurons can further be tested, and it can also be tested whether the neurons survive and enhance cognitive tasks. It is expected that newly generated neurons will survive and integrate into the central nervous system. The microbiomes of control and rifaximin-treated cohorts can be sequenced and compared to identify bacterial species, which may be enriched or eliminated. Cultivable probiotic species that differ can colonize Rett mice and effect on neurogenesis and cognition-related behaviors can be examined.

### Example 24

### Effects of Rifaximin Treatment on Inflammatory Markers

Rifaximin reduces the symptoms of IBD by altering the composition of microbiota favoring the propagation of health-promoting bacteria (Xu et al., 2014, Sartor,2016). The therapeutic benefits of rifaximin also include reducing systemic inflammation and the levels of cytokines such as IL-6 and TNF-α, which are elevated in the circulation and immune cells of Rett mice (Cronket al., 2015, Kang et al., 2016). Rifaximin reduces the inflammatory appearance of the GI tract in Rett mice raising the possibility that it may also affect the immune cells. Similar to what is proposed in Example 21, it is determined whether rifaximin changes the inflammatory phenotypes of the immune cells and the levels of inflammatory cytokines in the circulation. Changes in inflammatory phenotypes and/or inflammatory cytokines levels can be observed.

### REFERENCES

Each of the following references is hereby incorporated by reference in its entirety:

Ahluwalia V, Wade J B, Heuman D M, Hammeke T A, Sanyal A J, Sterling R K, Stravitz R T, Luketic V, Siddiqui M S, Puri. P. Fuchs M, Lennon M J, Kraft K A, Gilles H, White M B, Noble N A, Bajaj J S, (2014) Enhancement of Functional Connectivity, Working Memory and Inhibitory Control on Multi-modal Brain MR Imaging with Rifaximin in Cirrhosis: Implications for the Gut-Liver-Brain Axis. Metab Brain Dis. 29: 1017-1025.

Arboleya S, Watkins C, Stanton C, Ross R P, (2016) Gut Bifidobacteria Populations in Human Health and Aging. Front Microbiol. 7:1204.

Andrich J E, Wobben M, Klotz P, Goetze O, Saft C, (2009) Upper gastrointestinal findings in Huntington's disease: patients suffer but do not complain. J Neural Transm. 116:1607-1611.

Arrasate M, Finkbeiner 5, (2012) Protein aggregates in Huntington's disease. Exp Neurol 238:1-11.

Barbaro B A, Lukacsovich T, Agrawal N, Burke J, Bornemann D J, Purcell J M, Worthge S A, Caricasole A, Weiss A, Song W, Morozova O A, Colby D W, Marsh J L (2015) Comparative study of naturally occurring huntingtin fragments in Drosophila points to exon 1 as the most pathogenic species in Huntington's disease. Hum Mol Genet. 24:913-92.

Bartzokis G, Lu P H, Tishler T A, Fong S M, Oluwadara B, Finn J P, Huang D, Bordelon Y, Mintz J, Perlman S, (2007) Myelin breakdown and iron changes in Huntington's disease: pathogenesis and treatment implications. Neurochem Res. 32:1655-1664.

Bates G P, Dorsey R, Gusella J F, Hayden M R, Kay C, Leavitt B R, Nance M, Ross C A, Scahill R I, Wetzel R, Wild E J, Tabrizi S J, (2015) Huntington disease, Nat Rev Dis Primers. 1:15005.

Björkqvist M, Wild E J, Thiele J, Silvestroni A, Andre R, Lahiri N, Raibon E, Lee R V, Benn C L, Soulet D, Magnusson A, Woodman B, Landles C, Pouladi M A, Hayden M R, Khalili-Shirazi A, Lowdell M W, Brundin P, Bates G P, Leavitt B R, Moller T, Tabrizi S J (2008) A novel pathogenicpathway of immune activation detectable before clinical onset in Huntington's disease, J Exp Med. 205:1869-1877.

Bajaj J S, Heuman D M, Sanyal A J, Hylemon P B, Sterling R K, Stravitz R T, Fuchs M, Ridlon J M, Daita K. Monteith P, Noble N A, White M B, Fisher A, Sikaroodi M, Rangwala. H, Gilievet P M, (2013) Modulation of the metabiome by rifaximin in patients with cirrhosis and minimal hepatic encephalopathy. PLoS One. 8:e60042.

Bie B, Wu J, et al., (2014) Epigenetic suppression of neuroligin 1 underlies amyloid-induced memory deficiency. Nat Neurosci. 17:223-31.

Blanton L V, Charbonneau M R, Salih T, Barratt M J, Venkatesh S, Ilkaveya O, Subramanian S, Manary M J, Trehan I, Jorgensen J M, Fan Y M, Henrissat B, Leyn S A, Rodionov D A, Osterman A L, Maleta K M, Newgard C B, Ashorn P, Dewey K G, Gordon J I, (2016) Gut bacteria that prevent growth impairments transmitted by microbiota from malnourished children. Science 351pii: aad3311.

Buchovecky C M, Turley S D, et al., (2013) A suppressor screen in Mecp2 mutant mice implicates cholesterol metabolism in Rett syndrome. Nat Genet. 45:1013-24.

Carroll J B, Bates G P, Steffan J, Saft C, Tabrizi S J (2015) Treating the whole body in Huntington's disease. Lancet Neural. 14:1135-1142.

Chariot A, (2009) The NF-kappaB-independent functions of IKK subunits in immunity and cancer. Trends Cell Biol. 19:404-13.

Chen S G, Stribinskis V, Rane M J, Demuth D R, Gozal E, Roberts A M, Jagadapillai R, Liu R, Choe K, Shivakumar B, Son F, Jin S, Kerber R, Adame A, Masliah E, Friedland R P, (2016) Exposure to the Functional Bacterial Amyloid Protein Curli Enhances Alpha-Synuclein Aggregation in Aged Fischer 344 Rats and Caenorhabditis elegans. Sci Rep. 6:34477.

Chesnokova V, Pechnick RN, et al., (2016) Chronic peripheral inflammation, hippocampal neurogenesis, and behavior. Brain Behav Immun. pii: S0889-1591(16)30017-4.

Christian K M, Song Ming G L (2014). Functions and dysfunctions of adult hippocampal neurogenesis. Annu Rev Neurosci.37:243-262.

Chu H, Maztnanian S K, (2013) Innate immune recognition of the microbiota promotes host-microbial symbiosis. Nat Immunol. 14:668-75.

Chu H, Khosravi A, Kusumawardhani I P, Kwon A H, Vasconcelos A C, Cunha L D, Mayer A E, Shen Y, Wu W L, Kambal A, Targan S R, Xavier R J, Ernst P B, Green D R, McGovern D P, Virgin H W, Mazmanian S K (2016) Gene-microbiota interactions contribute to the pathogenesis of inflammatory bowel disease. Science 352:1116-20.

Dawood M Y, Lumley L A, Robison C L, Saviolakis G A, et al., (2004) Accelerated Barnes maze test in mice for assessment of stress effects on memory. Ann N Y Acad Sci. 1032:304-7.

Derecki N C, Crank J C, Lu Z, Xu E, et al., (2012) Wild-type microglia arrest pathology in a mouse model of Rett syndrome, Nature 484:105-9.

Dinan T G, Cryan J F, (2016) Gut Instincts: microbiota as a key regulator of brain development, ageing and neurodegeneration. J Physiol. 595:489-503.

Elder G A, (2015) Update on TBI and Cognitive Impairment in Military Veterans. Curr Neurol Neurosci Rep. 15: 68.

Evrensel A, Ceylan M E, (2016) Fecal Microbiota Transplantation and Its Usage in Neuropsychiatric Disorders, Clin Psychopharmacol Neurosci. 14:231-7.Gacias NI, Gaspari S, Santos P M, Tamburini S, Andrade M, Zhang F, Shen N, Tolstikov V, Kiebish M A, Dupree JL, Zachariou V, Clemente J C, Casaccia P, (2016) Microbiota-driven transcriptional changes in prefrontal cortex override genetic differences in social behavior. Elife. 5 pii: x13442.

Goyal M S, Venkatesh S. Milbrandt J, Gordon J I, Raichle M E, (2015) Feeding the brain and nurturing the mind: Linking nutrition and the gut microbiota to brain development. Proc Natl Acad Sci U S A. 112:14105-14112.

Fasano A, Bove F, Gabrielli M, Petracca M, Zocco M A, Ragazzoni E, Barbaro F, Piano C, Fortuna 5, Tortora A, Di Giacopo R, Campanale M, Gigante G, Lauritano E C, Navarra P, Marconi S, Gasbarrini A, Bentivoglio A R, (2013) The role of small intestinal bacterial overgrowth in Parkinson's disease. Mov Disord. 28:1241-1249.

Fedele V, Roybon L, Nordstrom U, Li J Y, Brundin P. Neurogenesis in the R6/2 mouse model of Huntington's disease is impaired at the level of NeuroD1. Neuroscience. 2011 Jan. 26; 173: 76-81.

Hao S, Tang B, et al., (2015) Forniceal deep brain stimulation rescues hippocampal memory in Rett syndrome mice. Nature 526:430-4.

Harusato A, Geem D, et al., (2016) Macrophage Isolation from the Mouse Small and Large Intestine, Methods Mol Biol. 1422:171-80.

Hashim H, Azmin S, Razlan H, Yahya N W, Tan H J, Manaf M R, Ibrahim N M, (2014) Eradication of Helicobacter pylori infection improves levodopa action, clinical symptoms and quality of life in patients with Parkinson's disease. PLoS one 9:el 12330.

Hoban A E, Stilling R M, Ryan F J, Shanahan F, Dinan T G, Claesson M J, Clarke G, Cryan J F, (2016) Regulation of prefrontal cortex myelination by the microbiota. Transl Psychiatry 6: e774.

Hsiao E Y, McBride S W, Chow J, Mazmanian S K, Patterson P H (2012) Modeling an autism risk factor in mice leads to permanent immune dysregulation. Proc Natl Acad Sci U S A. 109:12776-81.

Hsiao E Y, McBride S W, Hsien S, Sharon G, Hyde E R, McCue T, Codelli J A, Chow J, Reisman S E, Petrosino J F, Patterson P H, Mazmanian S K, (2013) Microbiota Modulate Behavioral and Physiological Abnormalities Associated with Neurodevelopmental Disorders. Cell 155:1451-1463.

Jiang S, Li C, et al., (2014) MeCP2 reinforces STAT3 signaling and the generation of effector CD4+ T cells by promoting miR-124-mediated suppression of SOCS5. Sci Signal. 7:ra25. Jirkof P, (2014) Burrowing and nest building behavior as indicators of well-being in mice. J Neurosci Methods 234:139-146.

Jirkof P, (2014) Burrowing and nest building behavior as indicators of well-being in mice. J Neurosei Methods. 234:139-46.

Johnston M, Blue M E, et al., (2015) Recent advances in understanding synaptic abnormalities in Rett syndrome. F1000Res. 4. pii: F1000 Faculty Rev-1490.

Kang D J, Kakiyama G, Betrapally N S, Herzog J, Nittono H, Hylemon P B, Zhou H, Carroll I, Yang J, Gillevet P M, Jiao C, Takei H, Pandak W M, Iida T, Heuman D M, Fan S, Fiehn O, Kurosawa T, Sikaroodi M, Sartor R B, Bajaj J S, (2016), Rifaximin Exerts Beneficial Effects Independent of its Ability to Alter Microbiota Composition, Clin Transl Gastroenterol. 7:e187.

Kishi N, MacDonald J L, et al., (2016) Reduction of aberrant NE-κB signalling ameliorates Rett syndrome phenotypes in Mecp2-null mice. Nat Commun. 7:10520.

Khoshnan A, Patterson P H., (2012) Elevated IKKα accelerates the differentiation of human neuronal progenitor cells and induces MeCP2-dependent BDNF expression. PLoS One. 7: e41794.

Khoshnan A, Ko J., Watkin E E, Paige L A, Reinhart P H, Patterson P H (2004) Activation of the IkappaB kinase complex and nuclear factor-kappaB contributes to mutant huntingtin neurotoxicity. J Neurosci 24,7999-8008

Khosravi A, Yáñez A, Price J G, Chow A, Merad M, Goodridge H S, Mazmanian S K (2015) Gut microbiota promote hematopoiesis to control bacterial infection. Cell Host Microbe 15:374-81.

Kok B, Foxton M R, Clough C, Shawcross D L, (2013) Rifaximin is an efficacious treatment for the Parkinsonian phenotype of hepatic encephalopathy. Hepatology 58:1516-1517.

Kyle S M, Saha P K, et al., (2016) MeCP2 co-ordinates liver lipid metabolism with the NCoR1/HDAC3 corepressor complex. Hum Mol Genet. [Epub ahead of print]

Li C, Jiang S. et al., (2014) MeCP2 enforces Foxp3 expression to promote regulatory T cells' resilience to inflammation. Proc Natl Acad Sci U S A. 111:E2807-16.

Lioy D T, Garg S K, et at, (2011) A role for glia in the progression of Rett's syndrome. Nature. 475:497-500.

Lombardi L M, Baker S A, et al., (2015) MECP2 disorders: from the clinic to mice and back. J Clin Invest. 125:2914-23.

Luczynski P, McVey Neufeld K A, Oriach C S, Clarke G, Dinan T G, Cryan J F, (2016) Growing up in a Bubble: Using Germ-Free Animals to Assess the Influence of the Gut Microbiota on Brain and Behavior. Int J Neuropsychopharmacol. pii: pyw020.

Maccaferri S, Vitali B, Klinder A, Kolida S, Ndagijimana M, Laghi L, Calanni F, Brigidi P, Gibson G R, Costabile A, (2010) Rifaximin modulates the colonic microbiota of patients with Crohn's disease: an in vitro approach using a continuous culture colonic model system. J Antimicrob Chemother. 65:2556-2565.

Marchesi J R, Adams D H, Fava F, Hermes G D, Hirschfield G M, Hold G, Quraishi M N, Kinross J. Smidt H, Tuohy K M, Thomas L V, Zoetendal E G, Hart A, (2016) The gut microbiota and host health: a new clinical frontier. Gut. 65:330-339.

Mayer E A, Knight R, Mazmanian S K, Cryan J F, Tillisch K (2014) Gut microbes and the brain: paradigm shift in neuroscience. J Neurosci. 34:15490-6.

Mazmanian S K, Liu C H, Tzianabos A O, Kasper D L (2005) An immunomodulatory molecule of symbiotic bacteria directs maturation of the host immune system. Cell 122:107-18,

Mencarelli A, Migliorati M, Barbanti M, Cipriani S. Palladino G, Distrutti E, Renga B, Fiorucci S, (2010) Pregnane-X-receptor mediates the anti-inflammatory activities of rifaximin on detoxification pathways in intestinal epithelial cells. Biochem Pharmacol. 80:1700-1707.

Minter M R, Zhang C, Leone V, Ringus D L, Zhang X, Oyler-Castrillo P, Musch M W, Liao F, Ward J F, Holtzman D M, Chang E B, Tanzi R E, Sisodia S S, (2016) Antibiotic-induced perturbations in gut microbial diversity influences neuro-inflammation and amyloidosis in a murine model of Alzheimer's disease. Sci Rep. 6:30028.

Motil K J, Caeg E, et al., (2012) Gastrointestinal and nutritional problems occur frequently throughout life in girls and women with Rett syndrome. J Pediatr Gastroenterol Nutr. 55:292-8.

Nettleman M, (015) Gulf War Illness: Challenges Persist. Trans Am Clin Climatol Assoc. 126:237-47.

O'Driscoll C M, Lima M P, et al., (2015) Methyl CpG binding protein 2 deficiency enhances expression of inflammatory cytokines by sustaining NF-κB signaling in myeloid derived cells. J Neuroimmunol. 283:23-9.

Ogbonnaya E S, Clarke G, Shanahan F, Dinan T G, Cryan J F, O'Leary O F, (2015) Adult Hippocampal Neurogenesis Is Regulated by the Microbiome. Biol Psychiatry 78: e7-9.

Patel J J, Rosenthal M D, et a (2016) The gut in trauma, Curr Opin Crit Care. 22:339-46.

Paulsen J S, (2011) Cognitive impairment in Huntington disease: diagnosis and treatment. Curr Neural Neurosci Rep. 11:474-483.

Ponziani F R, Scaldaferri F, Petito V, Paroni Sterbini F, Pecere S, Lopetuso L R, Palladini A, Gerardi V, Masucci L, Pompili M, Camrnarota G, Sanguinetti M, Gasbarrini A, (2016) The Role of Antibiotics in Gut Microbiota Modulation: The Eubiotic Effects of Rifaximin. Dig Dis. 34:269-278.

Ramocki M B, Zoghbi H Y (2008) Failure of neuronal homeostasis results in common neuropsychiatric phenotypes. Nature 455:912-8.

Rivière A, Selak M, et al., (2016) Bifidobacteria and Butyrate-Producing Colon Bacteria: Importance and Strategies for Their Stimulation in the Human Gut. Front Microbiol, 7: e979.

Rothhammer V, Mascanfroni I D, et al., (2016) Type I interferons and microbial metabolites of tryptophan modulate astrocyte activity and central nervous system inflammation via the aryl hydrocarbon receptor. Nat Med. [Epub ahead of print]

Round J L, Mazmanian S K, (2009) The gut microbiota shapes intestinal immune responses during health and disease. Nat Rev Immunol. 9:313-323.

Round J L, Lee S M, Li J, Tran G, Jabri B, Chatila T A, Mazmanian S K (2011) The Toll-like receptor 2 pathway establishes colonization by a commensal of the human microbiota. Science 332:974-7.

Sartor R B, (2016) The potential mechanisms of action of rifaximin in the management of inflammatory bowel diseases. Aliment Pharmacol Ther. 43 Suppl 1:27-36.

Sampson T R, Debelius J W, Thron T, Janssen S, Shastri G G, Ilhan Z E, Challis C, Schretter C E, Rocha S, Gradinaru V. Chesselet M F, Keshavarzian A, Shannon K M, Krajmalnik-Brown R, Wittung-Stafshede P, Knight R, Mazmanian S K, (2016) Gut Microbiota Regulate Motor Deficits and Neuroinflammation in a Model of Parkinson's Disease. Cell, 167:1469-1480.

Savignac H M, Tramulias M, Kiely B, Dinan T G, Cryan J F, (2015) Bifidobacteria modulate cognitive processes in an anxious mouse strain. Behav Brain Res. 287:59-72.

Scheperjans F, Aho V. Pereira P A, Koskinen K, Paulin L, Pekkonen E, Haapaniemi E, Kaakkola S, (2015) Gut microbiota are related to Parkinson's disease and clinical phenotype. Mov Disord. 30:350-358.

Sherwin E, Rea K, Dinan T G, Cryan J F, (2016) A gut microbiome) feeling about the brain. Curr Opin Gastmenterol. 32:96-102.

Southwell A L, Ko J, Patterson P H, (2009) Intrabody gene therapy ameliorates motor, cognitive, and neuropathological symptoms in multiple mouse models of Huntington's disease. J Neurosci, 2943:13589-13602.

Southwell A L, Smith-Dijak A, Kay C, Sepers M, Villanueva E B, Parsons M P, Xie Y, Anderson L, Felczak B, Waltl S, Ko S, Cheung D, Dal Cengio L, Slama R, Petoukhov E, Raymond L A, Hayden M R, (2016) An enhanced Q175 knock-in mouse model of Huntington disease with higher mutant huntingtin levels and accelerated disease phenotypes. Hum Mol Genet. 25:3654-3675.

Strati F, Cavalieri D, et al., (2016) Altered gut microbiota in Rett syndrome. Microbione 4:41.

Tai Y F, Pavese N, Gerhard A, Tabrizi S J, Barker R A, Brooks D J, Piccini P, (2007) Microglial activation in presymptomatic Huntington's disease gene carriers. Brain 130:1759-1766

Titova N, Padmakumar C, Lewis S J, Chaudhuri K R, (2016) Parkinson's: a syndrome rather than a disease? J Neural Transtn, [Epub ahead of print].

Tropea D, Giacometti E, Wilson N R, Beard C. McCurry C, Fu D D, et al., (2009) Partial reversal of Rett Syndrome-like symptoms in MeCP2 mutant mice. Proc Natl Acad Sci U S A 106:2029-34.

Van Gerven N. Klein R D, Huligren S J. Remaut H, (2015). Bacterial amyloid formation: structural insights into curli biogensis. Trends Microbiol. 23:693-706.

Veitch D P, Friedl K E, et al., (2013) Military risk factors for cognitive decline, dementia and Alzheimer's disease. Curr Alzheimer Res. 10:907-30.

Wang J, Wegener J E, Huang T W, Sripathy S, et al., (2015) Wild-type microglia do not reverse pathology in mouse models of Rett syndrome. Nature 521:E1-4.

Xu D, Gao et al., (2014) Rifaximin alters intestinal bacteria and prevents stress-induced gut inflammation and visceral hyperalgesia in rats. Gastroenterology 146:484-96.

Yang T, Ramocki M B, Neul J L, Lu W. Roberts L, Knight J, et al. (2012) Overexpression of methyl-CpG binding protein 2 impairs T(H)1 responses. Sci. Transl. Med. 4, 163ra158.

Yin X, Farin H F, van Es J H, Clevers H, Langer R et al., (2014) Niche-independent high-purity cultures of Lgr5(+) intestinal stem cells and their progeny. Nat Methods 11:106-12.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods, compositions, kits, and uses described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “ a system having at least one of A, B, or C” would include but not be limited to systems that have A alone, B alone, C alone, A and B together, A and C together, B and C together, and/or A, B, and C together, etc.), It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one of skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or articles, and so forth.

Wherever a method of using a composition or product combination (e.g., a composition or product combination comprising, consisting essentially of, or consisting of a bacteria and/or an antibiotic) is disclosed herein, the corresponding composition for use is also expressly contemplated. For example, for the disclosure of a method of reducing or preventing a symptom of HD in a selected subject, comprising administering an amount of a composition comprising Bactericides to the subject, the corresponding composition comprising Bacteroides for use in reducing the likelihood of, delaying the onset of, or ameliorating one or more symptoms associated with is also contemplated.

While various aspects and embodiments have been disclosed herein, other aspects and embodiments will be apparent to those of skill in the art. The various aspects and embodiments disclosed herein are for purposes of illustration and are not intended to be limiting, with the true scope and spirit being indicated by the following claims.

What is claimed is:

1. A composition or product combination comprising isolated bacteria that comprise at least two of: Actinobacteria bacteria, Tenericutes bacteria, or Bacteroides bacteria.

2. The composition or product combination of claim 1, comprising isolated Actinobacteria bacteria and isolated Bacteroides bacteria.

3. The composition or product combination of any one of claims 1-2, comprising the isolated Actinobacteria bacteria, wherein the Actinobacteria bacteria comprises Bifidobacteria.

4. The composition or product combination of any one of claims 1-3, comprising the isolated Bacteroides bacteria, wherein the Bacteroides bacteria are selected from the group consisting of: B. fragilis, B. ovatus, and B. thetaiotaomicron, or a combination of two or more of the listed bacteria.

5. The composition or product combination of any one of claims 1-4, comprising Actinobacteria bacteria, Tenericutes bacteria, and Bacteroides bacteria.

6. The composition or product combination of any one of claims 1-5, wherein the isolated bacteria comprise bacteria that map to an OTU that maps to a bacterium selected from the group consisting of Mesoplasma entomophilum, Lactobacillus taiwanensis, Pediococcus argentinicus, Bifidobacterium choerinum.

7. The composition or product combination of claim 6, wherein the bacteria map to an OTU when the bacteria comprise a 16S rRNA sequence of at least 100 nucleotides that is least 97% identical to a reference 16S rRNA sequence of the OTU.

8. The composition or product combination of any one of claims 1-7, comprising no more than 10⁴ cfu of Firmicuies bacteria,

9. The composition or product combination of any one of claims 1-8, further comprising a pharmaceutically acceptable excipient.

10. The composition or product combination of any one of claims 1-9, further comprising an antibiotic.

11. The composition or product combination of claim 10, wherein the antibiotic comprises rifaximin.

12. The composition or product combination of any one of claims 10-11, wherein the antibiotic is in a separate composition that is separate from the isolated bacteria.

13. The composition or product combination of any one of claims 1-12, wherein the isolated bacteria are in a single composition.

14. The composition or product combination of any one of claims 1-13, wherein the isolated bacteria are in separate compositions from each other.

15. The composition or product combination of any one of claims 1-14 for use in reducing the likelihood of, delaying the onset of, or ameliorating one or more symptoms associated with Rett syndrome or Huntington's disease.

16. A composition or product combination comprising:
an antibiotic; and
an isolated bacteria selected from the group consisting of: Actinobacieria bacteria, Tenericutes bacteria, and Bacteroides bacteria.

17. The composition or product combination of claim 16, comprising the isolated Actinobacteria bacteria, wherein the Actinobacteria bacteria comprises Bifidobacteria.

18. The composition or product combination of any one of claims 16-17, wherein the composition comprises no more than 10⁴ cfu of Firmicutes bacteria.

19. The composition or product combination of any one of claims 16-18, wherein the antibiotic comprises rifaximin.

20. The composition or product combination of any one of claims 16-19, wherein the antibiotic is in a separate composition that is separate from the isolated bacteria.

21. The composition or product combination of any one of claims 16-20, comprising a pharmaceutically acceptable excipient.

22. The composition or product combination of any one of claims 16-21 for use in reducing the likelihood of, delaying the onset of, or ameliorating one or more symptoms associated with Rett syndrome or Huntington's disease.

23. A method of reducing the likelihood of, delaying the onset of, or ameliorating one or more symptoms associated with Rett syndrome or Huntington's disease in a subject in need thereof, the method comprising administering to the subject a composition or product combination comprising one or more bacteria selected from the group consisting of: Actinobacteria bacteria, Tenericutes bacteria, and Bacteroides bacteria, or a combination of the listed bacteria.

24. The method of claim 23, further comprising selecting said subject as being within a class of subjects that should receive a composition for reducing the likelihood of, delaying the onset of, or ameliorating one or more symptoms associated with Rett syndrome or Huntington's disease

25. The method of any one of claims 23-24, wherein the method reduces the likelihood of, delays the onset of, or ameliorates the one or more symptoms associated with Huntington's disease, and wherein Bacteroides bacteria are administered to the subject.

26. The method of any one of claims 23-25, wherein the method reduces the likelihood of, delays the onset of, or ameliorates the one or more symptoms associated with Huntington's disease, and wherein the Actinobacteria bacteria are administered to the subject.

27. The method of claim 23, wherein the method reduces the likelihood of, delays the onset of, or ameliorates the one or more symptoms associated with Rett syndrome, and wherein the Actinobacteria bacteria and Bacteroides bacteria are administered to the subject.

28. The method of any one of claims 23-27, wherein the Actinobacteria bacteria and the Bacteroides bacteria are administered to the subject simultaneously or separately.

29. The method of any one of claims 23-28, wherein the Actinobacteria bacteria and the Tenericutes bacteria are administered to the subject simultaneously or separately.

30. The method of any one of claims 23-29, wherein the Actinobacteria bacteria comprise Bifidobacteria.

31. The method of any one of claims 23-30, wherein the Bacteroides bacteria are selected from the group consisting of: B. fragilis, B. ovatus, and B. thetaiotaomicron.

32. The method of any one of claims 23-30, wherein the bacteria comprises bacteria that map to an OTU that maps to a bacterium selected from the group consisting of Mesoplasma entomophilum, Lactobacillus taiwanensis, Pediococcus argentinicus, Bifidobacterium choerinum

33. The method of claim 31, wherein a bacteria maps to an OTU when the bacteria comprise a 16S rRNA sequence of at least 100 nucleotides that is least 97% identical to a reference 16S rRNA sequence of the OTU.

34. The method of any one of claims 23-33, wherein no more than 10⁴ cfu of Firmicutes bacteria is administered to the subject.

35. A method of reducing the likelihood of, delaying the onset of, or ameliorating one or more symptoms associated with Rett syndrome or Huntington's disease in a subject in need thereof, the method comprising administering an antibiotic to the subject.

36. The method of claim 35, further comprising selecting said subject as one being within a class of subjects that should receive a composition for reducing the likelihood of, delaying the onset of, or ameliorating one or more symptoms associated with Rett syndrome or Huntington's disease.

37. The method of any one of claims 35-36, wherein the antibiotic comprises rifaximin.

38. The method of any one of claims 35-37, wherein the one or more symptoms associated with Rett syndrome are reduced in likelihood, delayed in onset, or ameliorated.

39. The method of any one of claims 35-38, wherein the one or more symptoms associated with Huntington's disease are reduced in likelihood, delayed in onset, or ameliorated.

40. The method of any one of claims 35-39, wherein administering the antibiotic reduces a quantity of gut bacteria in the subject by at least 95%.

41. The method of any one of claims 35-39, further comprising administering a composition or product combination comprising isolated bacteria selected from the group consisting of: Actinobacteria bacteria, Tenericutes bacteria, and Bacteroides bacteria, or a combination of the listed bacteria to the subject.

42. The method of claim 41, wherein the isolated bacteria comprise bacteria that map to an OTU that maps to a bacterium selected from the group consisting of Mesoplasma entomophilum, Lactobacillus taiwanensis, Pediococcus argentinicus, Bifidobacterium choerinurn.

43. The method of claim 42, wherein a bacteria maps to an OTU when the bacteria comprise a 16S rRNA sequence of at least 100 nucleotides that is least 97% identical to a reference 16S rRNA sequence of the OTU.

44. The method of any one of claims 41-43, wherein the Actinobacteria bacteria and Bacteroides bacteria are administered to the subject.

45. The method of any one of claims 41-44, wherein the Actinobacteria bacteria are administered to the subject, and wherein the Actinobacteria bacteria comprises Bifidobacteria.

46. The method of any one of claims 41-45, wherein the Bacteroides bacteria are administered to the subject, wherein the Bacteroides bacteria are selected from the group consisting of: B. fragilis, B. ovatus, and B. thetaiotaomicron, or a combination of two or more of the listed bacteria.

47. The method of any one of claims 41-46, wherein the isolated bacteria are administered simultaneously with the antibiotic.

48. The method of claim 47, wherein the isolated bacteria are administered at a different time than the antibiotic.

49. The method of claims 48, wherein: (a) the antibiotic is administered prior to the bacteria; or (b) the bacteria is administered prior to the antibiotic.

50. The method of any one of claims 35-49, wherein no more than 10⁴ cfu of Firmicutes bacteria is administered to the subject.

51. A method of determining a profile of a sample of a subject, the method comprising detecting at least one of:
(a) a presence and/or level of a gut bacterium selected from the group consisting of: Tenericutes, Actinobacteria,and Firmicutes, or a combination of two or more of the listed bacteria;
(b) a serum level of a neurotransmitter selected from the group consisting of Choline, 5-HT, Tyrosine, Dopamine, and Epinepherine, or two or more of the listed neurotransmitters, or
(c) an expression level of a cholinergic gene selected from the group consisting of: Chrna2, Chrna7, Chrb4, Chrm1, Slc5a7, Chat, Ache, and Slc18a3, or two or more of the listed genes,
wherein the profile comprises the detected presence and/or levels of (a), (b), (c), (a) and (b), (a) and (c), (b) and (c), or (a) and (b) and (c).

52. The method of claim 51, wherein determining the profile comprises determining (a), wherein the sample comprises gut and/or feces material of the subject, and wherein a presence or elevated risk of Rett syndrome is indicated by lower levels of Tenericutes or Actinobacteria, or increased levels of Firmicutes, relative to levels present in a non-Rett control sample.

53. The method of any one of claims 51-52, wherein determining the profile comprises determining (b), wherein the sample comprises serum of the subject, and wherein a presence or elevated risk of Rett syndrome is indicated by:
higher levels of Choline, Tyrosine, and/or Dopamine compared to a control; and/or
lower levels of 5H-T and/or Epinepherine,
relative to levels present in a non-Rett control sample.

54. The method of any one of claims 51-52, wherein determining the profile comprises determining (c), wherein the sample comprises nucleic acids of the subject, and wherein a presence or elevated risk of Rett syndrome is indicated by lower expression levels of Chrna2, Chrna7, Chrb4, Chrm1, Slc5a7, Chat, Ache, Slc18a3, or two or more of the listed genes, relative to levels present in a non-Rett control sample.

55. The method of any one of claims 51-54, wherein the Actinobacteria comprise Bifidobacteria.
